SEQUENCE LISTING (65 sequences)

SEQ ID NO: 1 - RNA, 36 nt, synthetic construct
  source 1..36: mol_type = other RNA
gtctaaaccgacccaataatttctactgttgtagat

SEQ ID NO: 2 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 3 - RNA, 30 nt, synthetic construct
  source 1..30: mol_type = other RNA
cacccgtgcaaaattgcaggggtctaaaac

SEQ ID NO: 4 - RNA, 102 nt, synthetic construct
  source 1..102: mol_type = other RNA
ggcgcgtttattccattactttggagccagtcccagcgactatgtcgtatggacgaagcgcttatttatcggagagaaaccgataagtaaaacgcatcaaag

SEQ ID NO: 5 - RNA, 29 nt, synthetic construct
  source 1..29: mol_type = other RNA
cgcgatttactaaaaagggcggccattga

SEQ ID NO: 6 - RNA, 29 nt, synthetic construct
  source 1..29: mol_type = other RNA
acatatttacaacccgattctgatcgcac

SEQ ID NO: 7 - RNA, 29 nt, synthetic construct
  source 1..29: mol_type = other RNA
caagctattccccacagagcttcctctta

SEQ ID NO: 8 - RNA, 29 nt, synthetic construct
  source 1..29: mol_type = other RNA
atgatccgtggcatttacgatgagctcgt

SEQ ID NO: 9 - RNA, 29 nt, synthetic construct
  source 1..29: mol_type = other RNA
cacccgacgagtcttagccgcgataagca

SEQ ID NO: 10 - RNA, 29 nt, synthetic construct
  source 1..29: mol_type = other RNA
ccccccggctaaaattttgcgccgcgccc

SEQ ID NO: 11 - RNA, 29 nt, synthetic construct
  source 1..29: mol_type = other RNA
aattttttaacaacaccacaaaccccatc

SEQ ID NO: 12 - RNA, 29 nt, synthetic construct
  source 1..29: mol_type = other RNA
agcgtcctgtaatggggtaccgcgagaag

SEQ ID NO: 13 - RNA, 29 nt, synthetic construct
  source 1..29: mol_type = other RNA
cgccttttgcttcgcgtctaccatggcat

SEQ ID NO: 14 - RNA, 20 nt, synthetic construct
  source 1..20: mol_type = other RNA
tttaacaatcgtctcgtgga

SEQ ID NO: 15 - RNA, 20 nt, synthetic construct
  source 1..20: mol_type = other RNA
actttggcaagtaagcccgc

SEQ ID NO: 16 - RNA, 20 nt, synthetic construct
  source 1..20: mol_type = other RNA
gattgttgcacgggagaacc

SEQ ID NO: 17 - RNA, 20 nt, synthetic construct
  source 1..20: mol_type = other RNA
gaccaagatctggacgggtg

SEQ ID NO: 18 - RNA, 20 nt, synthetic construct
  source 1..20: mol_type = other RNA
gggtgtatgacacgttgtcg

SEQ ID NO: 19 - RNA, 20 nt, synthetic construct
  source 1..20: mol_type = other RNA
tgtttaacgacatatcgcca

SEQ ID NO: 20 - RNA, 20 nt, synthetic construct
  source 1..20: mol_type = other RNA
tttacttcggcttttacaag

SEQ ID NO: 21 - RNA, 20 nt, synthetic construct
  source 1..20: mol_type = other RNA
aaaggggtccttcgaacacg

SEQ ID NO: 22 - RNA, 20 nt, synthetic construct
  source 1..20: mol_type = other RNA
catacgggaacgcacatagt

SEQ ID NO: 23 - RNA, 20 nt, synthetic construct
  source 1..20: mol_type = other RNA
caactcatagagagttagcg

SEQ ID NO: 24 - RNA, 20 nt, synthetic construct
  source 1..20: mol_type = other RNA
cgtggtttagagaagcgcac

SEQ ID NO: 25 - RNA, 20 nt, synthetic construct
  source 1..20: mol_type = other RNA
cctctgaccttaattatagg

SEQ ID NO: 26 - RNA, 20 nt, synthetic construct
  source 1..20: mol_type = other RNA
atatatgccttaatgatggg

SEQ ID NO: 27 - RNA, 20 nt, synthetic construct
  source 1..20: mol_type = other RNA
gcggcgaattccaaacctgc

SEQ ID NO: 28 - RNA, 20 nt, synthetic construct
  source 1..20: mol_type = other RNA
gtgctcgtgcaaaacgggca

SEQ ID NO: 29 - RNA, 20 nt, synthetic construct
  source 1..20: mol_type = other RNA
aagagcatgcgaagaaaaaa

SEQ ID NO: 30 - RNA, 20 nt, synthetic construct
  source 1..20: mol_type = other RNA
actgccattacaaaacgcag

SEQ ID NO: 31 - RNA, 20 nt, synthetic construct
  source 1..20: mol_type = other RNA
tcaatatttttttcaagggt

SEQ ID NO: 32 - RNA, 20 nt, synthetic construct
  source 1..20: mol_type = other RNA
tccgcggaatagatatcatt

SEQ ID NO: 33 - RNA, 20 nt, synthetic construct
  source 1..20: mol_type = other RNA
tgcccattgatatccatgaa

SEQ ID NO: 34 - RNA, 20 nt, synthetic construct
  source 1..20: mol_type = other RNA
tgtggcgaaaaaacatctgc

SEQ ID NO: 35 - RNA, 29 nt, synthetic construct
  source 1..29: mol_type = other RNA
tttaagggcggttcgtttgttaagtagat

SEQ ID NO: 36 - RNA, 29 nt, synthetic construct
  source 1..29: mol_type = other RNA
tccggtctccacgcatctgtttggatgaa

SEQ ID NO: 37 - RNA, 29 nt, synthetic construct
  source 1..29: mol_type = other RNA
ttcagctttaatagcagttctacatgagt

SEQ ID NO: 38 - RNA, 29 nt, synthetic construct
  source 1..29: mol_type = other RNA
ggggacacgaccgccgcaacctgcagagg

SEQ ID NO: 39 - RNA, 29 nt, synthetic construct
  source 1..29: mol_type = other RNA
cccttttcgtacacacgtatcagcggggt

SEQ ID NO: 40 - RNA, 29 nt, synthetic construct
  source 1..29: mol_type = other RNA
tcaaaatgtttgaacatgctttttacttc

SEQ ID NO: 41 - RNA, 29 nt, synthetic construct
  source 1..29: mol_type = other RNA
tccgcgtttaaagggctattcgttgtttt

SEQ ID NO: 42 - RNA, 29 nt, synthetic construct
  source 1..29: mol_type = other RNA
ggcagctgctgaacagcctctgataattt

SEQ ID NO: 43 - RNA, 29 nt, synthetic construct
  source 1..29: mol_type = other RNA
ggcaaacgtaaatcgcgttcaaagttttc

SEQ ID NO: 44 - RNA, 20 nt, synthetic construct
  source 1..20: mol_type = other RNA
ggttcccttaacagggctat

SEQ ID NO: 45 - RNA, 20 nt, synthetic construct
  source 1..20: mol_type = other RNA
gtagatacagtcatacaacg

SEQ ID NO: 46 - RNA, 20 nt, synthetic construct
  source 1..20: mol_type = other RNA
ttgtttttccacgataacgt

SEQ ID NO: 47 - RNA, 20 nt, synthetic construct
  source 1..20: mol_type = other RNA
gccgtcggcggtggtaagaa

SEQ ID NO: 48 - RNA, 20 nt, synthetic construct
  source 1..20: mol_type = other RNA
gcgccatcacagagcccgtg

SEQ ID NO: 49 - RNA, 20 nt, synthetic construct
  source 1..20: mol_type = other RNA
tgtcacgtaatgttgttttg

SEQ ID NO: 50 - RNA, 20 nt, synthetic construct
  source 1..20: mol_type = other RNA
ggcaagcgacaggctcaaaa

SEQ ID NO: 51 - RNA, 20 nt, synthetic construct
  source 1..20: mol_type = other RNA
ccttcttccaatacttttag

SEQ ID NO: 52 - RNA, 20 nt, synthetic construct
  source 1..20: mol_type = other RNA
tgcacggttttctcctcggg

SEQ ID NO: 53 - RNA, 20 nt, synthetic construct
  source 1..20: mol_type = other RNA
gaggagcatagaatggtggt

SEQ ID NO: 54 - RNA, 20 nt, synthetic construct
  source 1..20: mol_type = other RNA
cccttggccatgaggctttt

SEQ ID NO: 55 - RNA, 20 nt, synthetic construct
  source 1..20: mol_type = other RNA
aaaccgttttaacgatttca

SEQ ID NO: 56 - RNA, 20 nt, synthetic construct
  source 1..20: mol_type = other RNA
gcccgacgccgttggttccc

SEQ ID NO: 57 - RNA, 20 nt, synthetic construct
  source 1..20: mol_type = other RNA
tgatgacccctcccagggag

SEQ ID NO: 58 - RNA, 20 nt, synthetic construct
  source 1..20: mol_type = other RNA
aatggcccttgaaattgtaa

SEQ ID NO: 59 - RNA, 20 nt, synthetic construct
  source 1..20: mol_type = other RNA
tcctcctttactgttagcat

SEQ ID NO: 60 - RNA, 20 nt, synthetic construct
  source 1..20: mol_type = other RNA
ccataagatctttcccatcc

SEQ ID NO: 61 - RNA, 20 nt, synthetic construct
  source 1..20: mol_type = other RNA
cttgatcctctcgggcctta

SEQ ID NO: 62 - protein, 1133 aa, African swine fever virus
MAYPELDAADFLQQLARRKEFKSLISPPVDQKELIRDLRAHFVQIGGPGCEKGGRAFFPCDPYASPFPSIKGLQLHNAQLFVQNFQNPNTPYSRLLLNWQTGTGKSIAAIAIARQFMNHYMNFIENAPWIFVVGFTRAIIQTEMLRRPELGFVSYKEVAELHRLLHIAKQSGSTTSVESRHLNGFVSTLKRRLTDRNRGGFFQFYGYKEFASKLFNITSKGEEKNFDVLSLFHRSDEAEDTLNENDISQFVQKISEAETNGLIRVNQKIMEQLRGGLLIADEIHNVYNIQERNNYGIALQYVLDAFPPHQAPRAVFMSATPVTGSVMEYVDLLNLLVPRHELPNGQPLQRQQLFDSSGHSVKWKKDALALVERLSTGRVSFLLDTNTNFYPERIFAGKMLSYKDETLPYLHFIECPMSEYQLETLKQLGPDPKISSNAYSIYDMVFPNPKFSKQTEPKAYGLFNSTETPTALSMASTDWLLENGVQIIEPSRRAPFNVSGSFLSLQPPTHISGLAFYSGKYTQMMKDILSIIRQGRGKILIYHNRVRMSGVLILQEILQSNGILNEVSSPVGTTRCSICAAIRDEHTHSDHQFIPVRFTILHSEIEPAVRERSLALFNASSNLEGHQLRILIGSKVIVEGLNFQAVRYEMIMSLPLDIPRLIQVFGRVVRKNSHMELPPSERNVTIYLYVSTTPDGGPELAKYAQKLKEYILIQEGDKALRKHAIDGFTNQIKIDKPMLESLPLSPSITPANVGATVLNTFEAYGYGEQEVKTISNIIISLFMARPVWTYSELWKAVSTPKLIQGITIDNKLFSEDNFALALISLCYSKNQCKELWIQNRLCTIMHVPAKPEHLYVAAVLNHKKEPVLDIETYIRDFQLPAMHSIRITKYLEHSQTKEPFQVLYEKFQKDFQDEPMEQVLIHYPASFHYTMLEALIIDNLAGMGALVEVYKKFFIAFSKKDIQPFPDIFKIISHVPGDDNTLVGYATEDSVRLITSREDKTWHEIPLYMLNINVKRKENDIVIGYMESKGKALKFKIRPPIQVLKKNEITDIRMLNRGAVCETRGREEQQKIADQLGISLNLTKISAIKLCLLIRNNLLQKEMEARNQPNGMQDGIRWFYLFNDKMPSLVHTS

SEQ ID NO: 63 - protein, 1192 aa, African swine fever virus
MEAFEISDFKEHAKKKSMWAGALNKVTISGLMGVFTEDEDLMALPIHRDHCPALLKIFDELIVNATDHERACHSKTKKVTYIKISFDKGVFSCENDGPGIPIAKHEQASLIAKRDVYVPEVASCFFLAGTNINKAKDCIKGGTNGVGLKLAMVHSQWAILTTADGAQKYVQQINQRLDIIEPPTITPSREMFTRIELMPVYQELGYAEPLSETEQADLSAWIYLRACQCAAYVGKGTTIYYNDKPCRTGSVMALAKMYTLLSAPNSTIHTATIKADAKPYSLHPLQVAAVVSPKFKKFEHVSIINGVNCVKGEHVTFLKKTINEMVIKKFQQTIKDKNRKTTLRDSCSNIFVVIVGSIPGIEWTGQRKDELSIAENVFKTHYSIPSSFLTSMTRSIVDILLQSISKKDNHKQVDVDKYTRARNAGGKRAQDCMLLAAEGDSALSLLRTGLTLGKSNPSGPSFDFCGMISLGGVIMNACKKVTNITTDSGETIMVRNEQLTNNKVLQGIVQVLGLDFNCHYKTQEERAKLRYGCIVACVDQDLDGCGKILGLLLAYFHLFWPQLIIHGFVKRLLTPLIRVYEKGKTMPVEFYYEQEFDAWAKKQTSLVNHTVKYYKGLAAHDTHEVKSMFKHFDNMVYTFTLDDSAKELFHIYFGGESELRKRELCTGVVPLTETQTQSIHSVRRIPCSLHLQVDTKAYKLDAIERQIPNFLDGMTRARRKILAGGVKCFASNNRERKVFQFGGYVADHMFYHHGDMSLNTSIIKAAQYYPGSSHLYPVFIGIGSFGSRHLGGKDAGSPRYISVQLASEFIKTMFPAEDSWLLPYVFEDGQRAEPEYYVPVLPLAIMEYGANPSEGWKYTTWARQLEDILALVRAYVDKDNPKHELLHYAIKHKITILPLRPSNYNFKGHLKRFGQYYYSYGTYDISEQRNIITITELPLRVPTVAYIESIKKSSNRMTFIEEIIDYSSSETIEILVKLKPNSLNRIVEEFKETEEQDSIENFLRLRNCLHSHLNFVKPKGGIIEFNSYYEILYAWLPYRRELYQKRLMREHAVLKLRIIMETAIVRYINESAELNLSHYEDEKEASRILSEHGFPPLNHTLIISPEFASIEELNQKALQGCYTYILSLQARELLIAAKTRRVEKIKKMQARLDKVEQLLQESPFPGASVWLEEIDAVEKAIIKGRNTQWKFH

SEQ ID NO: 64 - protein, 174 aa, African swine fever virus
MLTLIQGKKIVNDLRSRLAFEYNGQLIKILSKNIVAVGSLRREEKMLNDVDLLIIVPEKKLLKHVLPNIRIKDLSFSVKVCGERKCVLFIEWKKNTYQLDLFTALAEEKPYAVLHFTGPVSYLIRIRAALKKKNYKLNQYGLFKNQTLVPLKITTEKELIKELGFTYRIPKKRL

SEQ ID NO: 65 - DNA, 524 bp, African swine fever virus
  source 1..524: mol_type = genomic DNA
atgttaacgcttattcaaggaaaaaaaattgtaaatgacttacgctcccgacttgcgtttgaatataatggacaacttataaaaattttatcaaaaaacatcgttgctgttggtagcttaagacgcgaagagaaaatgcttaatgacgtggatcttcttattattgttccagaaaaaaaacttttaaaacacgtcctgcccaacattcgcataaaggatctttctttttctgtaaaagtctgcggagaacgaaagtgtgtactttttattgaatggaaaaaaaacacatatcaacttgatctttttacggccttagccgaggaaaagccatacgcagtacttcattttacgggtcccgtttcttatttaataagaattcgagctgcgttaaaaaaaaagaattataagctaaatcagtatggattatttaaaaatcaaactttagtacctctaaaaatcactactgaaaaagaacttattaaagaattaggatttacgtatcgcatacctaagaaacgtttata

(19) United States
(12) Patent Application Publication
(10) Pub. No.: US 2025/0263706 A1
(43) Pub. Date: Aug. 21, 2025

(54) COMPOSITIONS AND METHODS OF TREATING AFRICAN SWINE FEVER

(71) Applicant: Seek Labs, Inc., Salt Lake City, UT (US)

(72) Inventor: Dylan J. KEISER, Salt Lake City, UT (US)

(21) Appl. No.: 19/036,842

(22) Filed: Jan. 24, 2025

Related U.S. Application Data
(63) Continuation of application No. PCT/US2023/070959, filed on Jul. 25, 2023.
(60) Provisional application No. 63/392,081, filed on Jul. 25, 2022.

(51) Int. Cl. C12N 15/113; A61K 31/7105; A61K 38/46; A61P 31/20; C12N 9/22

(52) U.S. Cl. CPC C12N 15/1131; A61K 31/7105; A61K 38/465; A61P 31/20; C12N 9/226; C12N 2310/20

(57) ABSTRACT

The disclosure relates to compositions comprising and methods of administration to a mammal of single guide RNA (sgRNA), tracrRNA and/or crRNA used individually or in combination with one another or Cas system components in order to treat African Swine Fever Virus (ASFV). The disclosure also relates to systems for assaying therapeutically effective amounts of nucleic acid and protein that inhibit ASFV replication. This abstract is intended as a scanning tool for purposes of searching in the particular art and is not intended to be limiting of the present disclosure.

### CROSS-REFERENCE TO RELATED APPLICATIONS

This application is a Continuation of International Application No. PCT/US2023/070959, filed Jul. 25, 2023, which claims the benefit of U.S. Provisional Patent Application No. 63/392,081, filed on Jul. 25, 2022, each of which is incorporated by reference herein in its entirety.

### SEQUENCE LISTING

The instant application contains a Sequence Listing which has been submitted electronically in XML format and is hereby incorporated by reference in its entirety. Said XML copy, created on Aug. 17, 2023, is named 58557-706_301_SL.xml and is 61,472 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### TECHNICAL FIELD

In one aspect, the present disclosure is directed, to compositions, nucleic acids, pharmaceutical compositions comprising the same, kits comprising the same, and methods for preventing or treating African Swine Fever Virus (ASFV) infection by administering a nucleic acid encoding an RNA guide sequence, a trans-activating crispr RNA (tracrRNA) or functional fragments thereof, optionally, with a Cas protein or with a nucleic acid encoding a Cas protein. The present disclosure also relates to methods for preventing or treating African Swine Fever comprising degrading of an ASFV double-stranded DNA (dsDNA), an ASFV single-stranded RNA (ssRNA), or an ASFV dsDNA and an ASFV ssRNA by administering an ASFV-targeting CRISPR vector to a subject.

### BACKGROUND

Therapeutic genome editing has great potential to benefit a range of diseases. A key challenge is the efficient and clinically suitable delivery of genome editing biomacromolecules. The CRISPR (clustered regularly interspersed short palindromic repeats)/Cas9 system is a transforming and powerful genome editing tool. CRISPR/Cas9 comprises a short guide RNA (sgRNA) and an RNA-guided nuclease (e.g., a Cas9 protein). Cas9-sgRNA complex recognizes the protospacer-adjacent motif (PAM) and a 20-nucleotide sequence in the genome by Watson-Crick base pairing. Site-specific double-stranded DNA breaks (DSB) generated by Cas9 are repaired by endogenous cellular mechanisms, including homology-directed repair (HDR) or nonhomologous end-joining (NHEJ). The therapeutic potential of CRISPR/Cas9 requires safe and efficient delivery.

An ideal genome editing delivery system would limit the duration of exposure to editing machinery in order to minimize potential side effects. Cas9-sgRNA ribonucleoprotein (RNP)-based delivery of CRISPR has been tested for cell culture or local delivery in mouse inner ear cells, but these methods are not amenable for systemic in vivo delivery to target major organs such as the liver. Viral vehicles including the adeno-associated virus (AAV) have been used as the delivery agents for long-term CRISPR expression. However, spCas9, as the most commonly used form of Cas9, is difficult to fit in typical AAV constructs with strong promoters. A smaller form of Cas9 has been shown to be capable of being packaged into a single AAV construct. However, concerns regarding potential off-target effects remain if Cas9 is stably expressed by AAV delivery. Moreover, the T cell responses to AAV capsid can limit repeat dosing in patients. The long-term presence of Cas9, a protein from bacteria, in human tissue also increases the risk of immunogenicity. These limitations can be substantively addressed using non-viral delivery system. Previously, lipid nanoparticles (LNP) encapsulated Cas9 mRNA in combination with an AAV carrying an sgRNA and a repair template inducing efficient genome editing in the mouse liver.

African Swine Fever Virus (ASFV) has been a virus causing significant damage to the domesticated and commercial pig populations in Asia and Africa. Recently, ASFV has been detected in pig populations within the Dominican Republic, potentially threatening commercial and domesticated pig populations within North and South America. Current prophylactics or treatments for the virus remain unknown.

### SUMMARY

In accordance with the purpose(s) of the disclosure, as embodied and broadly described herein, the disclosure, in some embodiments, relates to compounds and compositions useful in the treatment of ASFV such as, for example, sgRNA or tracrRNA molecules designed to bind an ASFV target sequence and a Cas protein that cuts the target sequence leaving the virus diminished in an ability of replication, assembly, or replication and assembly or incapable of replication, assembly, or replication and assembly. The elimination of virus or increased inhibition of viral replication or virus production in host cells greatly improves outcomes for subject treated with such compositions. Furthermore, even alleviation of symptoms by administration of the disclosed compositions can significantly delay infection rates and decrease the spread of virus among a drift of pigs.

Provided herein are methods for treating ASFV infection in a subject in need thereof, the method comprising administering to the subject a therapeutically effective amount of a composition comprising: (i) a nucleic acid disclosed herein, or a pharmaceutically acceptable salt thereof; or (ii) a nucleic acid disclosed herein, or a pharmaceutically acceptable salt thereof and/or Cas protein, or pharmaceutically acceptable salts thereof. In some embodiments the nucleic acid sequence comprises any one or plurality of sequences from Table 2.

Also provided are methods of inhibiting ASFV infection in a subject exposed to ASFV disclosed herein, the method comprising administering to the subject a therapeutically effective amount of a composition comprising: (i) a nucleic acid disclosed herein, or a pharmaceutically acceptable salt thereof; or (ii) a nucleic acid disclosed herein, or a pharmaceutically acceptable salt thereof and/or Cas protein, or pharmaceutically acceptable salts thereof. Also provided are methods of inhibiting ASFV replication or assembly in a cell, the method comprising administering to the subject a therapeutically effective amount of a composition comprising: (i) a nucleic acid disclosed herein, or a pharmaceutically acceptable salt thereof; or (ii) a nucleic acid disclosed herein, or a pharmaceutically acceptable salt thereof and/or Cas protein, or pharmaceutically acceptable salts thereof. In some embodiments, the method further comprises a step of contacting or exposing the composition to a cell for a time period sufficient for association of the nucleic acid sequence to the ASFV DNA target sequence. In some embodiments, the methods of inhibiting or treatment further comprise a step of exposing the pharmaceutical composition disclosed herein to a viral genome in a cell for a time period sufficient for a CRISPR/Cas protein complex to form and excise one or more cuts in ASFV genome, such that the viral genomic DNA is left non-translational. In some embodiments, the aforementioned step is performed in the cytosol, but not the nucleus of the cell.

Also provided are kits comprising a composition comprising: (i) a nucleic acid disclosed herein, or a pharmaceutically acceptable salt thereof; or (ii) a nucleic acid disclosed herein, or pharmaceutically acceptable salt thereof and/or Cas protein, or pharmaceutically acceptable salts thereof; and one or more selected from: (a) instructions for treating ASFV; and (b) instructions for administering the composition.

The present disclosure relates to a nucleic acid sequence comprising at least one or a combination of domains from a 5′ to 3′ orientation: a DNA-binding domain, a Cas protein-binding domain, and a transcription terminator domain. The present disclosure relates to a nucleic acid sequence comprising at least one or a combination of domains from a 5′ to 3′ orientation: a DNA-binding domain, a Cas protein-binding domain, and a transcription terminator domain, wherein the DNA-binding domain comprises from about 1 to about 50 ribonucleotides or deoxyribonucleotides complementary to an ASFV target sequence. The present disclosure also relates to a nucleic acid sequence comprising at least one or a combination of domains from a 5′ to 3′ orientation: a DNA-binding domain, a Cas protein-binding domain, and a transcription terminator domain, wherein the DNA-binding domain comprises from about 1 to about 20 ribonucleotides or deoxyribonucleotides complementary to a DNA target sequence, wherein from about 1 to about 6 nucleotides are modified or non-natural nucleotides. In some embodiments, the pentose sugar or phosphodiester bond is modified. In some embodiments, the ASFV target sequence is ASFV helicase 1, helicase 2, helicase 3, topoisomerase 1, topoisomerase 2, topoisomerase 3, or polymerase. In some embodiments, the sgRNA or trRNA comprises a DNA-binding domain that is about 70% sequence identity to a nucleic acid sequence complementary to ASFV helicase 1, helicase 2, helicase 3, topoisomerase 1, topoisomerase 2, topoisomerase 3, or polymerase.

The present disclosure also relates to a nucleic acid sequence comprising at least one or a combination of domains from a 5′ to 3′ orientation: a DNA-binding domain, a Cas protein-binding domain, and a transcription terminator domain, wherein the DNA-binding domain comprises from about 1 to about 50 ribonucleotides or deoxyribonucleotides complementary to a DNA target sequence. In some embodiments, the sgRNA or trRNA comprises a DNA-binding domain that is about 70% sequence identity to a nucleic acid sequence complementary to ASFV helicase 1, helicase 2, helicase 3, topoisomerase 1, topoisomerase 2, topoisomerase 3, or polymerase and wherein the Cas-binding protein comprises at least about 70% sequence identity to Cas9, Cas12(a), Cas12a2, Cas13(d), or a functional fragment thereof. In some embodiments, the DNA-binding domain comprises a nucleic acid sequence that comprises at least about 70% sequence identity to SEQ ID NO:1 through SEQ ID NO:12, or a functional fragment or pharmaceutically acceptable salt thereof. In some embodiments, the DNA-binding domain comprises a nucleic acid sequence that comprises at least about 70% sequence identity to SEQ ID NO:1 through SEQ ID NO:12, or a functional fragment or pharmaceutically acceptable salt thereof.

The present disclosure relates to pharmaceutical compositions and kits comprising analogs and functional fragments of a nucleic acid sequence or sequences disclosed herein; and a pharmaceutically acceptable carrier. In some embodiments, the pharmaceutical composition comprises: (i) a nucleic acid disclosed herein, or pharmaceutically acceptable salt thereof; or (ii) a nucleic acid disclosed herein, or pharmaceutically acceptable salt thereof and/or Cas protein, or pharmaceutically acceptable salts thereof; and a pharmaceutically acceptable carrier. In some embodiments, the pharmaceutical composition has a therapeutically effective amount of (i) a nucleic acid disclosed herein, or pharmaceutically acceptable salt thereof; or (ii) a nucleic acid disclosed herein, or pharmaceutically acceptable salt thereof and/or Cas protein, or pharmaceutically acceptable salts thereof. The disclosure also relates to a pharmaceutical composition comprising a vector or liposome comprising an interior volume and an exterior surface, wherein the interior volume comprises: (i) a nucleic acid disclosed herein, or a pharmaceutically acceptable salt thereof; or (ii) a nucleic acid disclosed herein, or a pharmaceutically acceptable salt thereof and Cas protein, or pharmaceutically acceptable salts thereof. In some embodiments, the pharmaceutical composition comprises a first vector or first liposome comprising an interior volume and, an interior surface and an exterior surface, wherein the interior volume comprises: (i) a nucleic acid sequence disclosed herein, or a pharmaceutically acceptable salt thereof; or (ii) a nucleic acid sequence disclosed herein, or a pharmaceutically acceptable salt thereof and a pharmaceutically acceptable carrier; and (ii) a second vector or liposome comprising an interior volume and an exterior surface, wherein the interior volume comprises: (i) a Cas protein, or pharmaceutically acceptable salts thereof; or a nucleic acid sequence encoding a Cas protein, or pharmaceutically acceptable salt thereof.

The present disclosure also provides a composition comprising a nucleic acid sequence comprising at least 80% sequence identity to SEQ ID NO:1, and comprises at least one of the amino acid substitutions in SEQ ID NO: 1. In some embodiments, the nucleic acid sequence comprises at least 90% sequence identity to SEQ ID NO:1, and comprises at least one of the amino acid substitutions in SEQ ID NO: 1. In some embodiments, the nucleic acid sequence comprises at least 95% sequence identity to SEQ ID NO:1, and comprises at least one of the amino acid substitutions in SEQ ID NO: 1. In some embodiments, the nucleic acid sequence comprises at least 99% sequence identity to SEQ ID NO:1. In some embodiments, the nucleic acid sequence comprises at least 80% sequence identity to SEQ ID NO:2. In some embodiments, the nucleic acid sequence comprises at least 90% sequence identity to SEQ ID NO:2. In some embodiments, the nucleic acid sequence comprises at least 95% sequence identity to SEQ ID NO:2. In some embodiments, the nucleic acid sequence comprises at least 99% sequence identity to SEQ ID NO:2.

The present disclosure also provides a composition comprising a nucleic acid sequence comprising at least 80% sequence identity to SEQ ID NO:3. In some embodiments, the nucleic acid sequence comprises at least 90% sequence identity to SEQ ID NO:3. In some embodiments, the nucleic acid sequence comprises at least 95% sequence identity to SEQ ID NO:3. In some embodiments, the nucleic acid sequence comprises at least 99% sequence identity to SEQ ID NO:3. The present disclosure also provides a composition comprising a nucleic acid sequence comprising at least 80% sequence identity to SEQ ID NO:4. In some embodiments, the nucleic acid sequence comprises at least 90% sequence identity to SEQ ID NO:4. In some embodiments, the nucleic acid sequence comprises at least 95% sequence identity to SEQ ID NO:4. In some embodiments, the nucleic acid sequence comprises at least 99% sequence identity to SEQ ID NO:4.

In some embodiments, the nucleic acid sequence comprises one or a combination of: SEQ ID NO: 1, SEQ ID NO:2, SEQ ID NO:3, SEQ ID NO:4, SEQ ID NO:5, SEQ ID NO:6, SEQ ID NO:7, SEQ ID NO:9, SEQ ID NO:10, SEQ ID NO:11, SEQ ID NO:12, SEQ ID NO:13, SEQ ID NO:14, and SEQ ID NO: 15, or pharmaceutically acceptable salts thereof. In some embodiments, the DNA-binding region comprises at least about 70% or 80% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:9, SEQ ID NO:10, SEQ ID NO:11, SEQ ID NO:12, SEQ ID NO:13, SEQ ID NO:14, or SEQ ID NO:15 but is free of SEQ ID NO:1, SEQ ID NO:2, SEQ ID NO:3, SEQ ID NO:4, SEQ ID NO:5, SEQ ID NO:6, SEQ ID NO:7, SEQ ID NO:9, SEQ ID NO:10, SEQ ID NO:11, SEQ ID NO:12, SEQ ID NO:13, SEQ ID NO:14, SEQ ID NO: 15, or a pharmaceutically acceptable salt thereof. In some embodiments, the DNA-binding region of the nucleic acid sequence comprises at least about 70% or 80% sequence identity to a sequence on Table 2.

The present invention also provides vectors comprising any of the nucleic acids described above encoding any of the proteins described above. In some embodiments, the vector is a plasmid. In some embodiments, the vector is a retrovirus. In some embodiments, the pharmaceutical composition comprises a particle or viral pseudoparticle comprising the sgRNA, trRNA and/or Cas protein disclosed herein.

The disclosure also relates to methods of inhibiting viral replication or viral assembly comprising exposing a therapeutically effective amount of a composition or pharmaceutical composition disclosed here to a subject comprising ASFV. The disclosure also relates to methods of inhibiting viral replication or viral assembly comprising exposing a therapeutically effective amount of a composition or pharmaceutical composition disclosed herein to a cell. Still further embodiments relate to a method of treating a subject in need thereof with ASFV infection comprising administering to the subject a pharmaceutical composition disclosed herein. In some embodiments, the subject is a pig or minipig. In some embodiments, the cell is a mammalian cell. In some embodiments, the cell is a human cell or porcine cell.

In some embodiments, the disclosure relates to methods of inhibiting ASFV replication or assembly in a cell comprising administering to the cell a pharmaceutical composition disclosed herein. In some embodiments, the subject is a pig or minipig. In some embodiments, the cell is a mammalian cell. In some embodiments, the cell is a human cell or porcine cell.

The disclosure relates to a method of treating a pig infected with ASFV comprising administering to the pig a therapeutically effective amount of a pharmaceutical composition comprising (i) a nucleic acid disclosed herein, or a pharmaceutically acceptable salt thereof; or (ii) a nucleic acid disclosed herein, or a pharmaceutically acceptable salt thereof and Cas protein, or pharmaceutically acceptable salts thereof. In some embodiments, the pharmaceutical composition comprises a first vector or first liposome comprising an interior volume and, an interior surface and an exterior surface, wherein the interior volume comprises: (i) a nucleic acid sequence disclosed herein, or a pharmaceutically acceptable salt thereof; or (ii) a nucleic acid sequence disclosed herein, or a pharmaceutically acceptable salt thereof and a pharmaceutically acceptable carrier; and (ii) a second vector or liposome comprising an interior volume and an exterior surface, wherein the interior volume comprises: (i) a Cas protein, or pharmaceutically acceptable salts thereof; or a nucleic acid sequence encoding a Cas protein, or pharmaceutically acceptable salt thereof.

The disclosure further relates to a method of preventing ASFV replication or ASFV virus assembly in vivo or in vitro comprising administering to a cell, in vitro or in vivo, a therapeutically effective amount of a pharmaceutical composition comprising (i) a nucleic acid disclosed herein, or a pharmaceutically acceptable salt thereof; or (ii) a nucleic acid disclosed herein, or a pharmaceutically acceptable salt thereof and Cas protein, or pharmaceutically acceptable salts thereof. In some embodiments, the pharmaceutical composition comprises a first vector or first liposome comprising an interior volume and, an interior surface and an exterior surface, wherein the interior volume comprises: (i) a nucleic acid sequence disclosed herein, or a pharmaceutically acceptable salt thereof; or (ii) a nucleic acid sequence disclosed herein, or a pharmaceutically acceptable salt thereof and a pharmaceutically acceptable carrier; and (ii) a second vector or liposome comprising an interior volume and an exterior surface, wherein the interior volume comprises: (i) a Cas protein, or pharmaceutically acceptable salts thereof; or a nucleic acid sequence encoding a Cas protein, or pharmaceutically acceptable salt thereof. In some embodiments, the cell is from a mammal, and, in some embodiments, the cell is a porcine cell wherein the step of administering is performed to a pig.

Provided herein are methods for treating African Swine Fever in a subject in need thereof, the methods comprising administering to the subject a therapeutically effective amount of a first nucleic acid, or a pharmaceutically acceptable salt thereof; wherein the first nucleic acid comprises: i) a first nucleic acid sequence that comprises at least about 70% complementary to an endogenous African Swine Fever Virus (ASFV) nucleic acid sequence encoding an ASFV helicase, an ASFV DNA polymerase, or an ASFV topoisomerase; and ii) a second nucleic acid sequence encoding a Cas endonuclease protein or functional fragment thereof. In some embodiments, the Cas endonuclease protein or functional fragment thereof comprises Cas9 or Cas12a2. In some embodiments, the Cas endonuclease protein or functional fragment thereof comprises Cas12a2. In some embodiments, Cas12a2 comprises a nuclear localization signal (NLS). In some embodiments, Cas12a2 is lacking an NLS. In some embodiments, Cas12a2 is not substantially concentrated to a nucleus of a cell of the subject following the administering. In some embodiments, the first nucleic acid sequence comprises a sequence at least about 90%, 91%, 92%, 93%, 94%, 95%, 96%, or 97% identical to one or more sequences selected from SEQ ID NO: 1-61. In some embodiments, the first nucleic acid sequence comprises a sequence identical to one or more sequences selected from SEQ ID NO: 1-61. In some embodiments, ASFV viral load is decreased by at least about 5%, 6%, 7%, 8%, 9%, 10%, 11%, 12%, 13%, 14%, 15%, 16%, 18%, 20%, 22%, 24%, 25%, 27%, 29%, 30%, 32%, 35%, 37%, 40%, 42%, 45%, 47%, 50%, 52%, 55%, 57%, 59%, 60%, 62%, 65%, 67%, 70%, 72%, 75%, 77%, 80%, 85%, 90%, 95%, 96%, 97%, 98%, or 99%, less than one week after the administering. In some embodiments, ASFV virus being treated belongs to the family Asfarviridae. In some embodiments, the first nucleic acid sequence codes for a guide RNA (gRNA). In some embodiments, the gRNA activates a Cas protein. In some embodiments, the Cas protein is selected from the group consisting of Cas9, Cas12a, Cas12a2, Cas13b, and CasX. In some embodiments, the Cas protein functions as an endonuclease to cleave or digest one or more ASFV nucleic acids. In some embodiments, one or more ASFV nucleic acids comprise genomic DNA sequences identical to a portion of any one of an ASFV topoisomerase gene, an ASFV helicase gene, or an ASFV DNA polymerase gene. In some embodiments, one or more ASFV nucleic acids comprise single-stranded RNA sequences identical to any one of an ASFV topoisomerase mRNA, an ASFV helicase mRNA, or an ASFV DNA polymerase mRNA. In some embodiments, one or more ASFV nucleic acids comprise genomic DNA sequences identical to a portion of one or more ASFV topoisomerase genes. In some embodiments, one or more ASFV nucleic acids comprise genomic DNA sequences identical to a portion of one or more ASFV helicase genes. In some embodiments, one or more ASFV nucleic acids comprise genomic DNA sequences identical to a portion of one or more ASFV DNA polymerase genes. In some embodiments, one or more ASFV nucleic acids comprise single-stranded RNA sequences identical to a portion of one or more ASFV topoisomerase mRNA transcripts. In some embodiments, one or more ASFV nucleic acids comprise single-stranded RNA sequences identical to a portion of one or more ASFV helicase mRNA transcripts. In some embodiments, one or more ASFV nucleic acids comprise single-stranded RNA sequences identical to a portion of one or more ASFV DNA polymerase mRNA transcripts. In some embodiments, the gRNA and a Cas protein comprise a gene-binding moiety, wherein said gene binding moiety is configured to bind at least one essential gene of a virus belonging to the family Asfarviridae. In some embodiments, the ASFV topoisomerase comprises ASFV Topoisomerase II. In some embodiments, the ASFV helicase comprises an ASFV RNA helicase. In some embodiments, the ASFV DNA polymerase comprises an G1211R ASFV DNA polymerase or functional fragment thereof. In some embodiments, the Topoisomerase II is pi 192R or a fragment thereof. In some embodiments, the RNA helicase is QP509L, A859L, F105L, B92L, D1133LK, or Q706L. In some embodiments, the subject administered a therapeutically effective amount of a first nucleic acid is a mammal. In some embodiments, the subject administered a therapeutically effective amount of a first nucleic acid is a pig, a swine, a minipig, or a wild boar. In some embodiments, the subject administered a therapeutically effective amount of a first nucleic acid is a pig. In some embodiments, the pig species administered a therapeutically effective amount of a first nucleic acid is Sus scrofa, Sus ahenobarbus, Sus barbatus, Sus cebrifons, Sus celebensis, Sus oliveri, Sus philippensis, or Sus verrucosus. In some embodiments, the i) first nucleic acid sequence and the ii) second nucleic acid sequence are transcribed from a single vector. In some embodiments, the i) first nucleic acid sequence and the ii) second nucleic acid sequence are each transcribed from a separate vector. In some embodiments, the single vector or the separate vector comprise a plasmid. In some embodiments, method for treating African Swine Fever in a subject in need thereof further comprises a second nucleic acid that encodes one or more gRNAs, or one or more Cas proteins not encoded for within the first nucleic acid sequence. In some embodiments, ASFV topoisomerase, ASFV helicase, and ASFV DNA polymerase are each targeted by at least one nuclease, wherein at least one nuclease comprises at least three gene-binding moieties. In some embodiments, ASFV topoisomerase, ASFV helicase, and ASFV DNA polymerase are each targeted by at least one nuclease, wherein at least one nuclease comprises at least six gene-binding moieties. In some embodiments, ASFV topoisomerase, ASFV helicase, and ASFV DNA polymerase are each targeted by at least one nuclease, wherein at least one nuclease comprises at least nine gene-binding moieties. In some embodiments, ASFV topoisomerase, ASFV helicase, and ASFV DNA polymerase are each targeted by at least two nucleases using at least three gene-binding moieties. In some embodiments, ASFV topoisomerase, ASFV helicase, and ASFV DNA polymerase are each targeted by at least two nucleases using at least six gene-binding moieties. In some embodiments, ASFV topoisomerase, ASFV helicase, and ASFV DNA polymerase are each targeted by at least two nucleases using at least nine gene-binding moieties. In some embodiments, at least nine gene-binding moieties comprise at least nine distinct gRNAs.

Provided herein are vectors comprising a nucleic acid sequence encoding at least one programmable nuclease and a gRNA, wherein at least one programmable nuclease and the gRNA are configured to bind at least one essential viral gene of a virus from the family Asfarviridae. In some embodiments, the virus from the family Asfarviridae belongs to the genus Asfivirus. In some embodiments, the virus is African swine fever virus (ASFV). In some embodiments, at least one essential viral gene of the virus comprises an ASFV topoisomerase, an ASFV helicase, an ASFV DNA polymerase, or any combination thereof. In some embodiments, at least one essential viral gene of the virus comprises an ASFV topoisomerase. In some embodiments, at least one essential viral gene of the virus comprises an ASFV helicase. In some embodiments, at least one essential viral gene of the virus comprises an ASFV DNA polymerase. In some embodiments, at least one programmable nuclease comprises a Cas protein. In some embodiments, the Cas protein is selected from Cas9, Cas12a, Cas12a2, Cas13b, or CasX. In some embodiments, the Cas protein is Cas9 or a functional fragment thereof. In some embodiments, the Cas protein is Cas12a or a functional fragment thereof. In some embodiments, the Cas protein is Cas12a2 or a functional fragment thereof. In some embodiments, the Cas protein is Cas13b or a functional fragment thereof. In some embodiments, the Cas protein is CasX or a functional fragment thereof. In some embodiments, the Cas protein is Cas9. In some embodiments, the Cas protein is Cas12a. In some embodiments, the Cas protein is Cas12a2. In some embodiments, the Cas protein is Cas13b. In some embodiments, the Cas protein is CasX. In some embodiments, the gRNA comprises a sequence at least 90%, 91%, 92%, 93%, 94%, 95%, 96%, 97%, 98%, or 99% identical to a sequence selected from SEQ ID NO: 1-61. In some embodiments, the gRNA comprises a sequence selected from SEQ ID NO: 1-61. In some embodiments, the gRNA comprises at least two sequences selected from SEQ ID NO: 1-61. In some embodiments, the gRNA comprises at least six sequences selected from SEQ ID NO: 1-61. In some embodiments, the gRNA comprises at least twelve sequences selected from SEQ ID NO: 1-61. In some embodiments, the gRNA comprises at least eighteen sequences selected from SEQ ID NO: 1-61.

Provided herein are methods for preventing African Swine Fever in a subject in need thereof, the methods comprising administering to the subject a therapeutically effective amount of any vector described herein, or pharmaceutically acceptable salt thereof. In some embodiments, a risk of the subject developing acute ASFV infection is reduced following the administering. In some embodiments, the reduction in risk of developing acute ASFV infection is at least about 5%, 6%, 7%, 8%, 9%, 10%, 11%, 12%, 13%, 14%, 15%, 16%, 18%, 20%, 22%, 24%, 25%, 27%, 29%, 30%, 32%, 35%, 37%, 40%, 42%, 45%, 47%, 50%, 52%, 55%, 57%, 59%, 60%, 62%, 65%, 67%, 70%, 72%, 75%, 77%, 80%, 85%, 90%, 95%, 96%, 97%, 98%, or 99%, less than one week after the administering. In some embodiments, administering comprises systemic administration. In some embodiments, administering comprises oral administration. In some embodiments, oral administration comprises adding one or more components of a CRISPR system to a food source of the subject. In some embodiments, the subject administered a therapeutically effective amount of a vector described herein is a mammal. In some embodiments, the mammal is a pig, a swine, a minipig, or a wild boar.

Provided herein are kits comprising a pharmaceutical composition comprising the vector described herein, and instructions for use. In some embodiments, the instructions for use designate ASFV prophylaxis, ASFV treatment, or ASFV prophylaxis and ASFV treatment as indications in a subject in need of treatment. In some embodiments, the kit further comprises a drug delivery device.

Still other objects and advantages of the present disclosure will become readily apparent by those skilled in the art from the following detailed description, wherein it is shown and described only the preferred embodiments, simply by way of illustration of the best mode. As will be realized, the disclosure is capable of other and different embodiments, and its several details are capable of modifications in various obvious respects, without departing from the disclosure. Accordingly, the description is to be regarded as illustrative in nature and not as restrictive.

### BRIEF DESCRIPTIONS OF THE DRAWINGS

FIG. 1 depicts a schematic of the ASFV life cycle in a porcine cell infected by ASFV. The life cycle depicts infection, viral DNA being copied and translated, viral assembly and then release.

FIG. 2 depicts a schematic of the steps behind the conceived mechanism of action for the pharmaceutical compositions comprising the disclosed embodiments. The panel on the top left of the figure shows a liposome particle comprising Cas protein and one or plurality of disclosed nucleic acid sequences and a cell infected with ASFV. The top right panel depicts the liposome fusing to the infected cell and releasing its contents into the infected cell. The bottom left panel depicts the Cas protein machinery and the disclosed embodiments of nucleic acid sequences binding to the viral genome (indicated by looped line). The bottom right panel shows a cleavage of the viral genome after binding to the Cas protein and enzymatic action of the Cas protein and nucleic acid sequence in combination.

FIG. 3 depicts a more detailed depiction of the lower panels of FIG. 2. In FIG. 3, the Cas protein and nucleic acid comprising a DNA-binding domain are shown with granularity. The nucleic acid secondary structure is shown that associates with the Cas protein and the viral target DNA. The lower strand of viral DNA shows a cleavage event that disrupts the packaging and transcription of the viral genome, resulting in less functional virus being released by the infected cell.

FIG. 4A-4B depict in vitro experiments performed with 293T cells transfected with plasmid comprising nucleic acid sequence encoding GFP and the Cas protein with and without a nuclear localization signal. The cells were exposed to guide RNAs specific for the viral genomes with which the cells were infected or exposed. Results were assayed 72-hour post-infection with ASFV. FIG. 4A shows a graph of therapeutic transfection efficiency by assaying transfected plasmid expressing Green Fluorescent Protein (GFP) and quantified using flow cytometry. Transfection efficiency was measured at approximately 55%, 35%, and 42% for P18 VP2, P41 VP2, and P2400 VP respectively. These levels of GFP marker inform levels of Cas protein co-expressed as the GFP marker is linked to the Cas protein as expressed from these vectors. The amount of GFP in the system can be correlated to the total number of cells transfected in the experiment. FIG. 4B depicts the number of cells with detectable p72 protein from ASFV. This calculated percentage correlates to the amount of virus in the cells as compared to the number of cells that were successfully transfected (graph in FIG. 4A). P18 VP2 moderately reduced viral load, while P41 VP2 and P2400 VP2 treatment had a greater effect in reducing viral load. Treatments with P41 VP2 and P2400 VP2 each significantly reduced viral load compared to mock and untransfected control (p<0.05).

FIG. 5 depicts results from an in vitro experiment that normalizes the number of ASFV-infected cells treated and untreated with Cas protein and nucleic acid sequences comprising viral DNA targeting domains. Viral Load Percentage was calculated for nontreated and treated cells. The results depict about a 65% viral load reduction in treated experimental group of transfected cells.

FIG. 6 depicts two panels, a left and right panel showing prophetic experiments to be performed. The left panel depicts two sets of in vitro experiments in which a first set shows steps involved in a method of treating or exposing porcine cells to Cas protein/nucleic acid embodiments after exposure to ASFV. This schematic depicts an experiment to identify dosage for therapeutically effective amounts of treatment. The second set of experiments depicts steps involved in a method of treating or exposing porcine cells to Cas protein/nucleic acid embodiments before exposure to ASFV, which can identify dosage for prophylactically effective amounts of treatment. The right-hand side of experiments shows an identical series of steps except in actual animals (e.g., pigs), rather than porcine cells.

FIG. 7 depicts two sets of experiments used to identify treatment dosage in pigs as well as a timeline for running the experiments. As shown in FIG. 7 on the left, 15 Pigs are injected intramuscularly with Cas protein and nucleic acid embodiments at a dose 1 (DS1) or dose 2 (DS2). As shown in FIG. 7 on the right, 15 Pigs are injected intramuscularly with Cas protein and nucleic acid embodiments at a high dose (HD) or low dose (LD). Blood sampling is performed after therapeutic compositions are administered to the pigs to monitor viral load as compared to control animals (C) which are untreated with therapeutic compositions.

### DETAILED DESCRIPTION

### Overview

The present invention can be understood more readily by reference to the following detailed description of the disclosure and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such, they may vary. It is also to be understood that the terminology used herein is for the purpose of describing particular embodiments only and is not intended to be limiting. Although any methods and materials similar to or equivalent to those described herein can be used in the practice or testing of the present invention, example methods and materials are now described.

While embodiments of the present invention can be described and claimed in a particular statutory class, such as the system statutory class, this is for convenience only and one of skill in the art will understand that each embodiment of the present invention can be described and claimed in any statutory class. Unless otherwise expressly stated, it is in no way intended that any method or embodiment set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of embodiments described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### Compositions

The CRISPR/Cas or the CRISPR-Cas system (both terms are used interchangeably throughout this application) does not require the generation of customized proteins to target specific sequences but rather a single Cas protein (or CRISPR enzyme) can be programmed by a short RNA molecule to recognize a specific DNA target, in other words the Cas enzyme (such as a type II Cas9 protein) can be recruited to a specific DNA target using a short RNA molecule complementary to at least a portion of such specific ASFV DNA target. One aspect of the disclosure is a guide sequence. To utilize the CRISPR-Cas system effectively for genome editing without deleterious effects, it is critical to understand aspects of engineering and treatment of ASFV infection using these genome engineering tools, which are aspects of the disclosure.

In some embodiments, the disclosure relates to a nucleic acid sequence and compositions comprising the same. In another aspect, the disclosure relates to a nucleic acid sequence disclosed herein and compositions comprising the same with or without a vector that comprises a CRISPR enzyme or functional fragment thereof. In some embodiments, the nucleic acid sequence is a ribonucleic sequence or an sgRNA sequence that comprises from about 0% to about 99% modified nucleic acids in one, two or three domains which, in the 5′ to 3′ orientation, are: a DNA-binding domain, a Cas protein-binding domain, and a transcription terminator domain.

In some embodiments, the disclosure relates to a compositions comprising a guide sequence comprising, consisting essentially of, or consisting of a sequence that is 70%, 75%, 80%, 85%, 90%, 91%, 92%, 93%, 94%, 95%, 96%, 97%, 98%, 99% sequence identity to any one or combination of sequences disclosed herein, wherein the guide sequence comprises a fragment or variant of the sequences disclosed herein but possesses the same or substantially the same function as the full-length sequence disclosed herein. For example, in the case of a fragment or variant of a guide RNA disclosed herein that comprises modified nucleotides in the DNA-binding domain, in some embodiments, the variant or fragment would be functional insomuch as it would exceed or retain some or all of its capacity to bind DNA at that domain as compared to the full-length sequence.

In some embodiments, the DNA-binding domain is free of modifications in any one of its first 1, 2, 3, 4, 5 or more nucleotides on its 5′ end. In some embodiments the transcription terminator domain is free of modifications on any of its last 1, 2, 3, 4, 5 or more nucleotides on its 3′ end.

The disclosure relates to a nucleic acid sequence comprising at least one or a combination of domains from a 5′ to 3′ orientation: a DNA-binding domain, a Cas protein-binding domain, and a transcription terminator domain, wherein the DNA-binding domain comprises from about 1% to about 99% modified nucleotides and/or the transcription terminator domain comprises from about 1% to about 99% modified nucleotides. The disclosure relates to a nucleic acid sequence comprising a series of contiguous domains from a 5′ to 3′ orientation: a DNA-binding domain, a Cas protein-binding domain, and a transcription terminator domain, wherein the DNA-binding domain comprises from about 1% to about 99% modified nucleotides and/or the transcription terminator domain comprises from about 1% to about 99% modified nucleotides; and wherein the Cas protein-binding domain comprises from about 1% to about 99% modified nucleotides comprising one or a combination of the nucleotides in Table 2. The disclosure relates to a nucleic acid sequence consisting essentially of or consisting of a series of contiguous domains from a 5′ to 3′ orientation: a DNA-binding domain, a Cas protein-binding domain, and a transcription terminator domain, wherein the DNA-binding domain comprises from about 1% to about 99% modified nucleotides and/or the transcription terminator domain comprises from about 1% to about 99% modified nucleotides; and wherein the Cas protein-binding domain comprises from about 1% to about 99% modified nucleotides comprising one or a combination of the nucleotides in Table 2.

Any of the disclosed nucleic acid sequences may comprise any one or combination or set of modifications disclosed herein. In some embodiments, the guide nucleic acid, crRNA and/or tracr nucleic acid sequence comprises RNA, DNA, or combinations of both RNA and DNA. In some embodiments, the RNA, DNA, or combinations of both RNA and DNA, or a part thereof comprise a modified nucleobase or a modified sugar. Modifications to nucleotides are known in the art but include any of the disclosed modifications disclosed in the present application. Oligonucleotides particularly suited for the practice of one or more embodiments of the present disclosure comprise 2′-sugar modified oligonucleotides wherein one or more of the 2′-deoxy ribofuranosyl moieties of the nucleoside is modified with a halo, alkoxy, aminoalkoxy, alkyl, azido, or amino group. For example, the substitutions which may be independently selected from F, CN, CF3, OCF3, OCN, O-alkyl, S-alkyl, SMe, SO2Me, ONO2, NO2, NH3, NH2, NH-alkyl, OCH3═CH2 and OCCH. In each of these, alkyl is a straight or branched chain of C1 to C20, having unsaturation within the carbon chain. A preferred alkyl group is C1-C9 alkyl. A further preferred alkyl group is C5-C20 alkyl. A first group of substituents includes 2′-deoxy-2′-fluoro substituents. A further preferred group of substituents include C1 through C20 alkoxyl substituents. An additional group of substituents include cyano, fluoromethyl, thioalkoxyl, fluoroalkoxyl, alkylsulfinyl, alkylsulfonyl, allyloxy or alkeneoxy substituents.

“Cas binding domain” refers to a nucleic acid element or domain within a nucleic acid sequence or polynucleotide sequence that, in a biophysically effective amount, will bind or have an affinity for one or a plurality of proteins (or functional fragments thereof) encoded by one or a plurality of CRISPR-associated genes. In some embodiments, in the presence of a the one or a plurality of proteins (or functional fragments thereof) and a target sequence, the one or plurality of proteins and the nucleic acid element forms a biologically active CRISPR complex and/or can be enzymatically active on a target sequence. The terms “CRISPR-associated genes” refer to any nucleic acid that encodes a regulatory or expressible gene that regulates a component or encodes a component of the CRISPR system. In some embodiments, the terms “Cas-binding domain” or “Cas protein-binding domain” refers to a nucleic acid element or domain within a nucleic acid sequence or polynucleotide sequence that, in a biophysically effective amount, will bind to or have an affinity for one or a plurality of proteins in Table 2 (or functional fragments or variants thereof that are at least about 70, 75, 80, 85, 90, 95, 96, 97, 98, or 99% identical to the sequences disclosed in Table 2). In some embodiments, the Cas binding domain consists of no more than about 10, 11, 12, 13, 14, 15, 16, 17 18, 19, 20, 25, 30, 35, 40, 41, 42, 43, 44, 45, 50, 60, 70, 80, 90, 100, 110, 120, 130, 140, 150, 160, 170, 180, 190, 200, 210, 220, 230, 240, 250 or more nucleotides in length and comprises at least one sequence that is capable of forming a hairpin or duplex that partially associates or binds to a biologically active CRISPR system at a concentration and within microenvironment suitable for CRISPR system formation. In some embodiments, the composition or pharmaceutical compositions comprises one or a combination of sgRNA, crRNA, and/or tracrRNA that consists of no more than about 25, 30, 35, 40, 45, 50, 60, 70, 80, 90, 100, 110, 120, 130, 140, 150, 160, 170, 180, 190, 200, 210, 220, 230, 240, 250 or more nucleotides in length and comprises at least one sequence that is capable of forming a hairpin or duplex that partially associates or binds to a biologically active protein (or functional fragment thereof) of an amino acid sequence disclosed in Table 2 at a concentration and within microenvironment suitable for CRISPR system formation and CRISPR enzymatic activity on a target sequence. In some embodiments, the Cas protein is derived from the Cas9 family of Cas proteins or a functional fragment thereof. In some embodiments, the Cas protein is derived from the Cas12(a) family of Cas proteins or a functional fragment thereof. In some embodiments, the Cas protein is derived from the Cas12a2 family of Cas proteins or a functional fragment thereof. In some embodiments, the Cas protein is derived from the Cas13b family of Cas proteins or a functional fragment thereof. In some embodiments, the Cas protein is derived from the CasX family of Cas proteins or a functional fragment thereof.

The terms “transcription terminator domain” refers to a nucleic acid element or domain within a nucleic acid sequence (or polynucleotide sequence) that, in a biophysically effective amount, prevents bacterial transcription when the CRISPR complex is in a bacterial species and/or creates a secondary structure that stabilizes the association of the nucleic acid sequence to one or a plurality of Cas proteins (or functional fragments thereof) encoded by one or a plurality of CRISPR-associated genes such that, in the presence of the one or a plurality of proteins (or functional fragments thereof), the one or plurality of Cas proteins and the nucleic acid element forms a biologically active CRISPR complex and/or can be enzymatically active on a target sequence in the presence of such a target sequence and a DNA-binding domain. In some embodiments, the transcription terminator domain consists of no more than about 25, 30, 35, 40, 45, 50, 60, 70, 80, 90, 100, 110, 120, 130, 140, 150, 160, 170, 180, 190, 200, 210, 220, 230, 240, 250 or more nucleotides in length and comprises at least one sequence that is capable of forming a hairpin or duplex that partially drives association of the nucleic acid sequence (e.g., sgRNA, crRNA with tracrRNA, or other nucleic acid sequence) to a biologically active CRISPR complex at a concentration and microenvironment suitable for CRISPR complex formation.

The terms “DNA-binding domain” refer to an element or refers to a nucleic acid element or domain within a nucleic acid sequence or sgRNA that is complementary to a target sequence. In some embodiments, in a biophysically effective amount upstream from a Cas-binding domain, the DNA-binding domain will bind or have an affinity for one or a plurality of target nucleic acid sequences such that, in the presence of a biologically active CRISPR complex, one or plurality of Cas proteins can be enzymatically active on the target sequence. In some embodiments, the DNA binding domain comprises about 20, 25, 30, 35, 40, 45, 50, 60, 70, 80, 90, 100, 110, 120, 130, 140, 150, 160, 170, 180, 190, 200, 210, 220, 230, 240, 250 or more nucleotides in length and comprises at least one sequence that is capable of forming Watson Crick base pairs with a target sequence as part of a biologically active CRISPR system at a concentration and microenvironment suitable for CRISPR system formation. In some embodiments, the DNA binding domain consists of no more than about 20, 25, 30, 35, 40, 45, 50, 60, 70, 80, 90, 100, 110, 120, 130, 140, 150, 160, 170, 180, 190, 200, 210, 220, 230, 240, 250 or more nucleotides in length and comprises at least one sequence that is capable of forming Watson Crick base pairs with a target sequence as part of a biologically active CRISPR system at a concentration and microenvironment suitable for CRISPR system formation.

“CRISPR system” refers collectively to transcripts or synthetically produced transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a nucleic acid sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, the target sequence is a DNA polynucleotide and is referred to as a DNA target sequence. In some embodiments, a target sequence comprises at least three nucleic acid sequences that are recognized by a Cas-protein when the Cas protein is associated with a CRISPR complex or system which comprises at least one sgRNA or one tracrRNA/crRNA duplex at a concentration and within a microenvironment suitable for association of such a system. In some embodiments the target DNA comprises at least one or more proto-spacer adjacent motifs which sequences are known in the art and are dependent upon the Cas protein system being used in conjunction with the sgRNA or crRNA/tracrRNAs employed by this work. In some embodiments, the target DNA comprises NNG, where G is a guanine and N is any naturally occurring nucleic acid. In some embodiments the target DNA comprises any one or combination of NNG, NNA, GAA, NNAGAAW and NGGNG, where G is a guanine, A is adenine, and N is any naturally occurring nucleic acid or nucleotide.

In some embodiments, a target sequence is located in the nucleus or cytoplasm of a cell. In some embodiments, the target sequence may be within an organelle of a eukaryotic cell, for example, a mitochondrion or a chloroplast. A sequence or template that may be used for recombination into the targeted locus comprising the target sequences is referred to as an “editing template” or “editing polynucleotide” or “editing sequence”. In aspects of the disclosure, an exogenous template polynucleotide may be referred to as an editing template. In an aspect of the disclosure, the recombination is homologous recombination. In some embodiments, a composition disclosed herein comprises a recombination template. A recombination template may be a component of another vector as described herein, contained in a separate vector, or provided as a separate polynucleotide. In some embodiments, a recombination template is designed to serve as a template in homologous recombination, such as within or near a target sequence nicked or cleaved by a CRISPR enzyme (or equivalently a “Cas protein”) as a part of a CRISPR complex. A template polynucleotide may be of any suitable length, such as about or more than about 10, 15, 20, 25, 50, 75, 100, 150, 200, 500, 1000, or more nucleotides in length. In some embodiments, the template polynucleotide is complementary to a portion of a polynucleotide comprising the target sequence. When optimally aligned, a template polynucleotide might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

In further embodiments of the present disclosure, the individual nucleotides of the oligonucleotides of the disclosure are connected via phosphorus linkages. Phosphorus linkages include phosphodiester, phosphorothioate and phosphorodithioate linkages. In one preferred embodiment of this disclosure, nuclease resistance is conferred on the oligonucleotides by utilizing phosphorothioate internucleoside linkages.

In another embodiment, the disclosure provides a cell or a vector comprising one of the sgRNAs of the disclosure or functional fragments thereof. The cell may be an animal cell or a plant cell. In some embodiments, the cell is a mammalian cell, such as a human cell.

In one aspect, the disclosure provides a vector system comprising one or more vectors. In some embodiments, the system comprises: (a) a synthetic guide sequence comprising at least one of the nucleic acid sequences disclosed herein, wherein the guide sequence directs sequence-specific binding of a CRISPR complex to a target sequence in a eukaryotic cell (such as a human or a porcine cell), wherein the CRISPR complex comprises a CRISPR enzyme complexed with (1) the guide sequence that is hybridized to the target sequence, and, optionally (2) a tracr mate sequence that is hybridized to a tracr sequence; and (b) a first regulatory element “operatively linked” to an enzyme-coding sequence encoding said CRISPR enzyme comprising a nuclear localization signal; wherein expressible components (the enzyme-coding sequence and the tracr sequences) are located on the same or different vectors of the system. Within a recombinant expression vector, “operatively linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). In some embodiments, the system comprises: (a) a synthetic guide sequence comprising at least one of the nucleic acid sequences disclosed herein, wherein the guide sequence directs sequence-specific binding of a CRISPR complex to a target sequence in a eukaryotic cell (such as a human or a porcine cell), wherein the CRISPR complex comprises a CRISPR enzyme complexed with (1) the guide sequence that is hybridized to the target sequence, and, optionally (2) a tracr mate sequence that is hybridized to a tracr sequence; and (b) a first regulatory element operatively linked to an enzyme-coding sequence encoding said CRISPR enzyme; wherein said CRISPR enzyme does not comprise a nuclear localization signal; wherein expressible components (the enzyme-coding sequence and the tracr sequences) are located on the same or different vectors of the system.

In some embodiments, component (a) further comprises the tracr sequence downstream of the tracr mate sequence under the control of a tracr regulatory element. In some embodiments, component (a) further comprises one or more additional guide sequences operatively linked to the tracr regulatory element, wherein when expressed, each the additional guide sequences direct sequence specific binding of a CRISPR complex to a different target sequence in a eukaryotic cell. In some embodiments, the system comprises the tracr sequence under the control of its own, second regulatory element, such as a polymerase III promoter. In some embodiments, the tracr sequence exhibits at least 50%, 60%, 70%, 80%, 90%, 95%, or 99% of sequence complementarity along the length of the tracr mate sequence when optimally aligned. Determining optimal alignment is within the purview of one who is skilled in the art. For example, there are publicly and commercially available alignment algorithms and programs such as, but not limited to, ClustalW, Smith-Waterman in matlab, Bowtie, Geneious, Biopython and SeqMan. In some embodiments, the CRISPR complex comprises one or more nuclear localization signals of sufficient strength to drive accumulation of said CRISPR complex in a detectable amount in the nucleus of a eukaryotic cell. Without wishing to be bound by theory, it is believed that a nuclear localization signal is not necessary for CRISPR complex activity in eukaryotes, but that including such sequences with coupled to particular Cas enzymes enhances activity of the system, especially as to targeting nucleic acid molecules in the nucleus. In some embodiments, the CRISPR enzyme is a type II CRISPR system enzyme. In some embodiments, the CRISPR enzyme is a Cas9 enzyme. In some embodiments, the Cas9 enzyme is S. pneumoniae, S. pyogenes, or S. thermophilus Cas9, and may include mutated Cas9 derived from these organisms. The enzyme may be a Cas9 homolog or ortholog. In some embodiments, the CRISPR enzyme is a Cas12(a) enzyme. In some embodiments, the CRISPR enzyme is a Cas12a2 enzyme. In some embodiments, the CRISPR enzyme is a Cas13b enzyme. In some embodiments, the CRISPR enzyme is a CasX enzyme. In some embodiments, the CRISPR enzyme is codon-optimized for expression in a eukaryotic cell. In some embodiments, the CRISPR enzyme directs cleavage of one or two strands at the location of the target sequence. In some embodiments, the CRISPR enzyme lacks DNA strand cleavage activity. In some embodiments, the first regulatory element is a polymerase III promoter. In some embodiments, the second regulatory element is a polymerase II promoter. In some embodiments the CRISPR enzyme is chosen from one or combination of Table Y.

In some embodiments, the CRISPR complex does not comprise one or more nuclear localization signals and said CRISPR complex accumulates in a detectable amount in the cytosol of a eukaryotic cell. Without wishing to be bound by theory, it is believed that excluding one of more NLS for CRISPR complex activity in eukaryotes for particular Cas enzymes (e.g., Cas12a2 and functional derivatives thereof) may enhance activity of the system, especially as to targeting nucleic acid molecules in the cytosol. Unlike some other Cas nucleases, once activated, Cas12a2 indiscriminately degrades targeted double stranded DNA, targeted single-stranded DNA and targeted single-stranded RNA. In some embodiments, Cas12a2 is ideally suited for targeting ASFV because: (i) it can be programmed to indiscriminately destroy DNA upon detection of ASFV RNA; (ii) is a multi-turnover enzyme instead of single turnover like Cas9 and Cas12a, and; (iii) because ASFV is a DNA virus the multi-turnover nature of Cas12a2 DNase activity would be essential to degrade the viral genomic material of ASFV.

In some embodiments, the CRISPR enzyme is a type II CRISPR system enzyme. In some embodiments, the CRISPR enzyme is a Cas12(a) enzyme. In some embodiments, the CRISPR enzyme is a Cas12a2 enzyme. In some embodiments, the CRISPR enzyme is a Cas13b enzyme. In some embodiments, the CRISPR enzyme is a CasX enzyme. In some embodiments, the CRISPR enzyme is codon-optimized for expression in a eukaryotic cell. In some embodiments, the CRISPR enzyme directs cleavage of one or two strands at the location of the target sequence. In some embodiments, the CRISPR enzyme lacks DNA strand cleavage activity. In some embodiments, the first regulatory element is a polymerase III promoter. In some embodiments, the second regulatory element is a polymerase II promoter. In some embodiments the CRISPR enzyme is chosen from one or a combination of Cas proteins listed in Table 1. In some embodiments, the CRISPR enzyme comprises one or more functional fragments of one or a combination of Cas proteins listed in Table 1. In some embodiments, the CRISPR enzyme comprises one of more Cas proteins comprising an amino acid sequence at least about 90%, 91%, 92%, 93%, 94%, 95%, 96%, 97%, 98%, or 99% identical to the amino acid sequence of one or more Cas proteins listed in Table 1. In some embodiments, the CRISPR enzyme comprises one of more Cas proteins comprising the amino acid sequence of one or more Cas proteins listed in Table 1. In some embodiments, the CRISPR enzyme comprises one of more Cas proteins comprising the amino acid sequence of CasX (Cas12e).

TABLE 1
Cas protein types Paired with Accession Number

| Cas Protein | Accession Number |
|---|---|
| Cas12a2 | Genbank: KIM12007.1 |
| Cas9 | Genbank: AZQ25086 |
| Cas13d | NCBI Reference Sequence: WP_117939725.1 |
| Cas12a | Genbank: ATB19154.1 |

In another embodiment, the disclosure provides a cell or a vector comprising one of the sgRNAs of the disclosure or functional fragments thereof. The cell may be an animal cell or a plant cell. In some embodiments, the cell is a mammalian cell, such as a human cell. In some embodiments, the cell is a mammalian cell, such as a porcine cell.

The disclosure relates to nucleic acid molecules comprising nucleic acid sequences that encode a Cas protein or functional fragment thereof. In some embodiments, the disclosure relates to a vector comprising a nucleic acid sequence that encodes a Cas protein or functional fragment thereof operatively linked to a regulatory sequence. In some embodiments, the vector comprises a nucleic acid sequence that encodes a Cas protein or functional fragment thereof, operatively linked to a regulatory sequence.

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). In some embodiments, the viral vector used to transport another nucleic acid to which it has been linked comprises an Adeno-associated virus (AAV) vector. In some embodiments, the AAV vector is selected from AAV1, AAV2, AAV3, AAV4, AAV5, AAV6, AAV7, AAV8, AAV9, or AAVrh10. In some embodiments, the viral vector used to transport another nucleic acid to which it has been linked comprises a lentiviral vector. In some embodiments, the vector used to transport another nucleic acid to which it has been linked comprises an exosome. In some embodiments, the vector used to transport another nucleic acid to which it has been linked comprises a red blood cell extracellular vesicle (RBCEV).

Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids.

Another aspect of the disclosure relates to a composition comprising a nucleic acid disclosed herein and one or a plurality of recombinant expression vectors. Generally, the disclosure relates to compositions comprising a synthetic guide sequence and one or a plurality of recombinant expression vectors. Recombinant expression vectors can comprise a nucleic acid of the disclosure in a form suitable for expression of the nucleic acid in a host cell, which means that the recombinant expression vectors include one or more regulatory elements, which may be selected on the basis of the host cells to be used for expression, that is operatively linked to the nucleic acid sequence to be expressed.

The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY, 185, Academic Press, San Diego, Calif (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol Ill promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and HI promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydro folate reductase promoter, the β-actin promoter, the phosphoglycerol kinase (PGK) promoter, and the EFla promoter. Also encompassed by the term “regulatory element” are enhancer elements, such as WPRE; CMV enhancers; the R-U5′ segment in LTR of HTLV-1 (Mol. Cell. Biol., Vol. 8(1), p. 466-472, 1988); SV40 enhancer; and the intron sequence between exons 2 and 3 of rabbit 3-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, e.g., the level of expression desired. One or more nucleic acid sequences and one or more vectors can be introduced into host cells to thereby form complexes with other cellular or non-natural compounds, produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Compositions of the disclosure in nucleotide sequences comprising percent sequence identity to any DNA-binding region disclosed herein. In some embodiments, nucleic acid sequences comprise from about 1 to about 100 modifications at recited position or across several nucleotides. Nucleic acid sequences or oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine. 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo uracil), 4-thiouracil, 8-halo, 8-amino. 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the disclosure. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2-O-methoxyethyl sugar modifications. Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502177; 5,525,711; 5,552,540; 5,587,469; 5,594,121, 5,596,091; 5,614,617; 5,681,941, and 5,750,692, each of which is herein incorporated by reference in its entirety.

Another modification of the oligonucleotides of the disclosure involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), palmityl moiety (Mishra et al., Biochim. Biophys. Acta, 995, 1264, 229-237), or an octadecylamine or hexylamino-carbonyl-oxycholesterol moiety (Crooke et al., J. Pharmacol. Exp. Ther., 1996, 277, 923-937).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254.469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference in its entirety.

It is not necessary for all positions in a given compound to be uniformly modified, and in fact more than one of the aforementioned modifications may be incorporated in a single sequence or compound or even at a single nucleoside or functional group within one or a plurality of positions within a nucleoside or an oligonucleotide.

Oligonucleotides of the present disclosure also relate to guide sequences comprising a one or a combination of: a DNA-binding domain, a Cas protein-binding domain, and a transcription terminator domain, and one or more targeting domains. As used herein “targeting domains” may be oligonucleotides, amino acid sequences, sugar moieties, lipid moieties or hybrids of any of the foregoing that are responsible for directing transformation or transfection or anchoring of the guide sequence disclosed herein into a cell of choice that comprises a target sequence. Creation of such chimeric molecules can be synthetically manufactured by known chemical arts.

For example, GalNAc-conjugated modifications are know % n to direct oligonucleotides to liver cells. Modifications, such as GalNAc-conjugated modification, may be made to any one or combination of oligonucleotides disclosed herein with automated solid phase synthesis, similar to the solid phase synthesis that produced unconjugated oligonucleotides. During the synthesis of GalNAc-conjugated oligonucleotides, the phosphoramidite monomers are sequentially coupled to a GalNAc conjugate which is covalently linked to a solid support. The synthesis of GalNAc conjugates and GalNAc conjugate solid support is described, for example in U.S. Pat. No. 8,106,022, which is herein incorporated by reference in its entirety for the description of the synthesis of carbohydrate-containing conjugates, including conjugates comprising one or more GalNAc moieties, and of the synthesis of conjugate covalently linked to solid support.

The disclosure also relates to synthesizing one or a plurality of oligonucleotides, such as sgRNA molecules. 2′-deoxy-2′-modified nucleosides of adenine, guanine, cytosine, thymidine and certain analogs of these nucleobases may be prepared and incorporated into oligonucleotides via solid phase nucleic acid synthesis. Novel oligonucleotides can be assayed for their hybridization properties and their ability to resist degradation by nucleases compared to the unmodified oligonucleotides. Initially, small electronegative atoms or groups can be selected because they would not be expected to sterically interfere with required Watson-Crick base pair hydrogen bonding (hybridization). However, electronic changes due to the electronegativity of the atom or group in the 2-position may profoundly affect the sugar conformation.

In some embodiments, nucleic acid molecules encoding the disclosed nucleic acid sequences, or salts thereof, are substantially isolated. Partial separation can include, for example, a composition enriched in the compound of the disclosure. Substantial separation can include compositions containing at least about 50%, at least about 60%, at least about 70%, at least about 80%, at least about 90%, at least about 95%, at least about 97%, or at least about 99% by weight of the compound of the disclosure, or salt thereof. Methods for isolating compounds and their salts are routine in the art, but include, as a non-limiting example, mini-prep, maxi-prep or column separation of nucleic acid molecules from cells after lysing those cells comprising a plurality of nucleic acids.

In some embodiments, nucleic acid molecules encoding the disclosed nucleic acid sequences, or salts thereof, are substantially isolated. Partial separation can include, for example, a composition enriched in the compound of the disclosure. Substantial separation can include compositions containing at least about 50%, at least about 60%, at least about 70%, at least about 80%, at least about 90%, at least about 95%, at least about 97%, or at least about 99% by weight of the compound of the disclosure, or salt thereof. Methods for isolating compounds and their salts are routine in the art, but include, as a non-limiting example, mini-prep, maxi-prep or column separation of nucleic acid molecules from cells after lysing those cells comprising a plurality of nucleic acids. Table 2 denotes Guide RNA sequences (paired in various combinations of repeat/tracrRNA sequences with Guide RNA spacer sequences) for use in combination with the specified Cas protein to target ASFV dsDNA in specific ASFV genes. In some embodiments, Guide RNA sequences (paired in various combinations of repeat/tracrRNA sequences with Guide RNA spacer sequences) for use in combination with the specified Cas protein target ASFV ssRNA transcribed from specific ASFV genes comprise the nucleic acid for use in the CRISPR system. In some embodiments, Guide RNA sequences (paired in various combinations of repeat/tracrRNA sequences with Guide RNA spacer sequences) for use in combination with the specified Cas protein target ASFV dsDNA in specific ASFV genes and target ASFV ssRNA transcribed from specific ASFV genes for use in the CRISPR system.

TABLE 2
Guide RNA Sequences comprising a repeat/tracrRNA and a Spacer RNA
paired with a Cas protein paired to target specific ASFV Target DNA or RNA sequences for
CRISPR-mediated targeted endonuclease activity

| | ASFV | | | Cas |
|---|---|---|---|---|
| Pairing | Target | Repeat/tracrRNA | Spacer | Protein |
| 1 | Helicase 1 | GUCUAAACCGACCCAA | CGCGAUUUACUAAA | Cas12a(2) |
| | | UAAUUUCUACUGUUG | AAGGGCGGCCAUUG | |
| | | UAGAU | A | |
| | | (SEQ ID NO: 1) | (SEQ ID NO: 5) | |
| 2 | Helicase 2 | GUCUAAACCGACCCAA | ACAUAUUUACAACC | Cas12a(2) |
| | | UAAUUUCUACUGUUG | CGAUUCUGAUCGCA | |
| | | UAGAU | C | |
| | | (SEQ ID NO: 1) | (SEQ ID NO: 6) | |
| 3 | helicase 3 | GUCUAAACCGACCCAA | CAAGCUAUUCCCCA | Cas12a(2) |
| | | UAAUUUCUACUGUUG | CAGAGCUUCCUCUU | |
| | | UAGAU | A | |
| | | (SEQ ID NO: 1) | (SEQ ID NO: 7) | |
| 4 | topoisomerase | GUCUAAACCGACCCAA | AUGAUCCGUGGCAU | Cas12a(2) |
| | 1 | UAAUUUCUACUGUUG | UUACGAUGAGCUCG | |
| | | UAGAU | U | |
| | | (SEQ ID NO: 1) | (SEQ ID NO: 8) | |
| 5 | topoisomerase | GUCUAAACCGACCCAA | CACCCGACGAGUCU | Cas12a(2) |
| | 2 | UAAUUUCUACUGUUG | UAGCCGCGAUAAGC | |
| | | UAGAU | A | |
| | | (SEQ ID NO: 1) | (SEQ ID NO: 9) | |
| 6 | topoisomerase | GUCUAAACCGACCCAA | CCCCCCGGCUAAAA | Cas12a(2) |
| | 3 | UAAUUUCUACUGUUG | UUUUGCGCCGCGCC | |
| | | UAGAU | C | |
| | | (SEQ ID NO: 1) | (SEQ ID NO: 10) | |
| 7 | polymerase 1 | GUCUAAACCGACCCAA | AAUUUUUUAACAAC | Cas12a(2) |
| | | UAAUUUCUACUGUUG | ACCACAAACCCCAU | |
| | | UAGAU | C | |
| | | (SEQ ID NO: 1) | (SEQ ID NO: 11) | |
| 8 | polymerase 2 | GUCUAAACCGACCCAA | AGCGUCCUGUAAUG | Cas12a(2) |
| | | UAAUUUCUACUGUUG | GGGUACCGCGAGAA | |
| | | UAGAU | G | |
| | | (SEQ ID NO: 1) | (SEQ ID NO: 12) | |
| 9 | polymerase 3 | GUCUAAACCGACCCAA | CGCCUUUUGCUUCG | Cas12a(2) |
| | | UAAUUUCUACUGUUG | CGUCUACCAUGGCA | |
| | | UAGAU | U | |
| | | (SEQ ID NO: 1) | (SEQ ID NO: 13) | |
| 10 | polymerase | GUUUUAGAGCUAGAA | UUUAACAAUCGUCU | Cas9 |
| | | AUAGCAAGUUAAAAU | CGUGGA | |
| | | AAGGCUAGUCCGUUA | (SEQ ID NO: 14) | |
| | | UCAACUUGAAAAAGU | | |
| | | GGCACCGAGUCGGUGC | | |
| | | (SEQ ID NO: 2) | | |
| 11 | polymerase | GUUUUAGAGCUAGAA | ACUUUGGCAAGUAA | Cas9 |
| | | AUAGCAAGUUAAAAU | GCCCGC | |
| | | AAGGCUAGUCCGUUA | (SEQ ID NO: 15) | |
| | | UCAACUUGAAAAAGU | | |
| | | GGCACCGAGUCGGUGC | | |
| | | (SEQ ID NO: 2) | | |
| 12 | polymerase | GUUUUAGAGCUAGAA | GAUUGUUGCACGGG | Cas9 |
| | | AUAGCAAGUUAAAAU | AGAACC | |
| | | AAGGCUAGUCCGUUA | (SEQ ID NO: 16) | |
| | | UCAACUUGAAAAAGU | | |
| | | GGCACCGAGUCGGUGC | | |
| | | (SEQ ID NO: 2) | | |
| 13 | topoisomerase | GUUUUAGAGCUAGAA | GACCAAGAUCUGGA | Cas9 |
| | | AUAGCAAGUUAAAAU | CGGGUG | |
| | | AAGGCUAGUCCGUUA | (SEQ ID NO: 17) | |
| | | UCAACUUGAAAAAGU | | |
| | | GGCACCGAGUCGGUGC | | |
| | | (SEQ ID NO: 2) | | |
| 14 | topoisomerase | GUUUUAGAGCUAGAA | GGGUGUAUGACACG | Cas9 |
| | | AUAGCAAGUUAAAAU | UUGUCG | |
| | | AAGGCUAGUCCGUUA | (SEQ ID NO: 18) | |
| | | UCAACUUGAAAAAGU | | |
| | | GGCACCGAGUCGGUGC | | |
| | | (SEQ ID NO: 2) | | |
| 15 | topoisomerase | GUUUUAGAGCUAGAA | UGUUUAACGACAUA | Cas9 |
| | | AUAGCAAGUUAAAAU | UCGCCA | |
| | | AAGGCUAGUCCGUUA | (SEQ ID NO: 19) | |
| | | UCAACUUGAAAAAGU | | |
| | | GGCACCGAGUCGGUGC | | |
| | | (SEQ ID NO: 2) | | |
| 16 | helicase | GUUUUAGAGCUAGAA | UUUACUUCGGCUUU | Cas9 |
| | | AUAGCAAGUUAAAAU | UACAAG | |
| | | AAGGCUAGUCCGUUA | (SEQ ID NO: 20) | |
| | | UCAACUUGAAAAAGU | | |
| | | GGCACCGAGUCGGUGC | | |
| | | (SEQ ID NO: 2) | | |
| 17 | helicase | GUUUUAGAGCUAGAA | AAAGGGGUCCUUCG | Cas9 |
| | | AUAGCAAGUUAAAAU | AACACG | |
| | | AAGGCUAGUCCGUUA | (SEQ ID NO: 21) | |
| | | UCAACUUGAAAAAGU | | |
| | | GGCACCGAGUCGGUGC | | |
| 18 | helicase | GUUUUAGAGCUAGAA | CAUACGGGAACGCA | Cas9 |
| | | AUAGCAAGUUAAAAU | CAUAGU | |
| | | AAGGCUAGUCCGUUA | (SEQ ID NO: 22) | |
| | | UCAACUUGAAAAAGU | | |
| | | GGCACCGAGUCGGUGC | | |
| | | (SEQ ID NO: 2) | | |
| 19 | scrambled | GUUUUAGAGCUAGAA | CAACUCAUAGAGAG | Cas9 |
| | | AUAGCAAGUUAAAAU | UUAGCG | |
| | | AAGGCUAGUCCGUUA | (SEQ ID NO: 23) | |
| | | UCAACUUGAAAAAGU | | |
| | | GGCACCGAGUCGGUGC | | |
| | | (SEQ ID NO: 2) | | |
| 20 | scrambled | GUUUUAGAGCUAGAA | CGUGGUUUAGAGAA | Cas9 |
| | | AUAGCAAGUUAAAAU | GCGCAC | |
| | | AAGGCUAGUCCGUUA | (SEQ ID NO: 24) | |
| | | UCAACUUGAAAAAGU | | |
| | | GGCACCGAGUCGGUGC | | |
| | | (SEQ ID NO: 2) | | |
| 21 | scrambled | GUUUUAGAGCUAGAA | CCUCUGACCUUAAU | Cas9 |
| | | AUAGCAAGUUAAAAU | UAUAGG | |
| | | AAGGCUAGUCCGUUA | (SEQ ID NO: 25) | |
| | | UCAACUUGAAAAAGU | | |
| | | GGCACCGAGUCGGUGC | | |
| | | (SEQ ID NO: 2) | | |
| 22 | helicase | CACCCGUGCAAAAUUG | CGCGAUUUACUAAA | Cas13d |
| | | CAGGGGUCUAAAAC | AAGGGCGGCCAUUG | |
| | | (SEQ ID NO: 3) | A | |
| | | | (SEQ ID NO: 5) | |
| 23 | helicase | CACCCGUGCAAAAUUG | ACAUAUUUACAACC | Cas13d |
| | | CAGGGGUCUAAAAC | CGAUUCUGAUCGCA | |
| | | (SEQ ID NO: 3) | C | |
| | | | (SEQ ID NO: 6) | |
| 24 | helicase | CACCCGUGCAAAAUUG | CAAGCUAUUCCCCA | Cas13d |
| | | CAGGGGUCUAAAAC | CAGAGCUUCCUCUU | |
| | | (SEQ ID NO: 3) | A | |
| | | | (SEQ ID NO: 7) | |
| 25 | topoisomerase | CACCCGUGCAAAAUUG | AUGAUCCGUGGCAU | Cas13d |
| | | CAGGGGUCUAAAAC | UUACGAUGAGCUCG | |
| | | (SEQ ID NO: 3) | U | |
| | | | (SEQ ID NO: 8) | |
| 26 | topoisomerase | CACCCGUGCAAAAUUG | CACCCGACGAGUCU | Cas13d |
| | | CAGGGGUCUAAAAC | UAGCCGCGAUAAGC | |
| | | (SEQ ID NO: 3) | A | |
| | | | (SEQ ID NO: 9) | |
| 27 | topoisomerase | CACCCGUGCAAAAUUG | CCCCCCGGCUAAAA | Cas13d |
| | | CAGGGGUCUAAAAC | UUUUGCGCCGCGCC | |
| | | (SEQ ID NO: 3) | C | |
| | | | (SEQ ID NO: 10) | |
| 28 | polymerase | CACCCGUGCAAAAUUG | AAUUUUUUAACAAC | Cas13d |
| | | CAGGGGUCUAAAAC | ACCACAAACCCCAU | |
| | | (SEQ ID NO: 3) | C | |
| | | | (SEQ ID NO: 11) | |
| 29 | polymerase | CACCCGUGCAAAAUUG | AGCGUCCUGUAAUG | Cas13d |
| | | CAGGGGUCUAAAAC | GGGUACCGCGAGAA | |
| | | (SEQ ID NO: 3) | G | |
| | | | (SEQ ID NO: 12) | |
| 30 | polymerase | CACCCGUGCAAAAUUG | CGCCUUUUGCUUCG | Cas13d |
| | | CAGGGGUCUAAAAC | CGUCUACCAUGGCA | |
| | | (SEQ ID NO: 3) | U | |
| | | | (SEQ ID NO: 13) | |
| 31 | helicase | GGCGCGUUUAUUCCA | AUAUAUGCCUUAAU | CasX |
| | | UUACUUUGGAGCCAG | GAUGGG | |
| | | UCCCAGCGACUAUGUC | (SEQ ID NO: 26) | |
| | | GUAUGGACGAAGCGC | | |
| | | UUAUUUAUCGGAGAG | | |
| | | AAACCGAUAAGUAAA | | |
| | | ACGCAUCAAAG | | |
| | | (SEQ ID NO: 4) | | |
| 32 | helicase | GGCGCGUUUAUUCCA | GCGGCGAAUUCCAA | CasX |
| | | UUACUUUGGAGCCAG | ACCUGC | |
| | | UCCCAGCGACUAUGUC | (SEQ ID NO: 27) | |
| | | GUAUGGACGAAGCGC | | |
| | | UUAUUUAUCGGAGAG | | |
| | | AAACCGAUAAGUAAA | | |
| | | ACGCAUCAAAG | | |
| | | (SEQ ID NO: 4) | | |
| 33 | helicase | GGCGCGUUUAUUCCA | GUGCUCGUGCAAAA | CasX |
| | | UUACUUUGGAGCCAG | CGGGCA | |
| | | UCCCAGCGACUAUGUC | (SEQ ID NO: 28) | |
| | | GUAUGGACGAAGCGC | | |
| | | UUAUUUAUCGGAGAG | | |
| | | AAACCGAUAAGUAAA | | |
| | | ACGCAUCAAAG | | |
| | | (SEQ ID NO: 4) | | |
| 34 | topoisomerase | GGCGCGUUUAUUCCA | AAGAGCAUGCGAAG | CasX |
| | | UUACUUUGGAGCCAG | AAAAAA | |
| | | UCCCAGCGACUAUGUC | (SEQ ID NO: 29) | |
| | | GUAUGGACGAAGCGC | | |
| | | UUAUUUAUCGGAGAG | | |
| | | AAACCGAUAAGUAAA | | |
| | | ACGCAUCAAAG | | |
| | | (SEQ ID NO: 4) | | |
| 35 | topoisomerase | GGCGCGUUUAUUCCA | ACUGCCAUUACAAA | CasX |
| | | UUACUUUGGAGCCAG | ACGCAG | |
| | | UCCCAGCGACUAUGUC | (SEQ ID NO: 30) | |
| | | GUAUGGACGAAGCGC | | |
| | | UUAUUUAUCGGAGAG | | |
| | | AAACCGAUAAGUAAA | | |
| | | ACGCAUCAAAG | | |
| | | (SEQ ID NO: 4) | | |
| 36 | topoisomerase | GGCGCGUUUAUUCCA | UCAAUAUUUUUUUC | CasX |
| | | UUACUUUGGAGCCAG | AAGGGU | |
| | | UCCCAGCGACUAUGUC | (SEQ ID NO: 31) | |
| | | GUAUGGACGAAGCGC | | |
| | | UUAUUUAUCGGAGAG | | |
| | | AAACCGAUAAGUAAA | | |
| | | ACGCAUCAAAG | | |
| | | (SEQ ID NO: 4) | | |
| 37 | polymerase | GGCGCGUUUAUUCCA | UCCGCGGAAUAGAU | CasX |
| | | UUACUUUGGAGCCAG | AUCAUU | |
| | | UCCCAGCGACUAUGUC | (SEQ ID NO: 32) | |
| | | GUAUGGACGAAGCGC | | |
| | | UUAUUUAUCGGAGAG | | |
| | | AAACCGAUAAGUAAA | | |
| | | ACGCAUCAAAG | | |
| | | (SEQ ID NO: 4) | | |
| 38 | polymerase | GGCGCGUUUAUUCCA | UGCCCAUUGAUAUC | CasX |
| | | UUACUUUGGAGCCAG | CAUGAA | |
| | | UCCCAGCGACUAUGUC | (SEQ ID NO: 33) | |
| | | GUAUGGACGAAGCGC | | |
| | | UUAUUUAUCGGAGAG | | |
| | | AAACCGAUAAGUAAA | | |
| | | ACGCAUCAAAG | | |
| | | (SEQ ID NO: 4) | | |
| 39 | polymerase | GGCGCGUUUAUUCCA | UGUGGCGAAAAAAC | CasX |
| | | UUACUUUGGAGCCAG | AUCUGC | |
| | | UCCCAGCGACUAUGUC | (SEQ ID NO: 34) | |
| | | GUAUGGACGAAGCGC | | |
| | | UUAUUUAUCGGAGAG | | |
| | | AAACCGAUAAGUAAA | | |
| | | ACGCAUCAAAG | | |
| | | (SEQ ID NO: 4) | | |
| 40 | polymerase | GUCUAAACCGACCCAA | UUUAAGGGCGGUUC | Cas12a(2) |
| | | UAAUUUCUACUGUUG | GUUUGUUAAGUAG | |
| | | UAGAU | AU | |
| | | (SEQ ID NO: 1) | (SEQ ID NO: 35) | |
| 41 | polymerase | GUCUAAACCGACCCAA | UCCGGUCUCCACGC | Cas12a(2) |
| | | UAAUUUCUACUGUUG | AUCUGUUUGGAUGA | |
| | | UAGAU | A | |
| | | (SEQ ID NO: 1) | (SEQ ID NO: 36) | |
| 42 | polymerase | GUCUAAACCGACCCAA | UUCAGCUUUAAUAG | Cas12a(2) |
| | | UAAUUUCUACUGUUG | CAGUUCUACAUGAG | |
| | | UAGAU | U | |
| | | (SEQ ID NO: 1) | (SEQ ID NO: 37) | |
| 43 | topoisomerase | GUCUAAACCGACCCAA | GGGGACACGACCGC | Cas12a(2) |
| | | UAAUUUCUACUGUUG | CGCAACCUGCAGAG | |
| | | UAGAU | G | |
| | | (SEQ ID NO: 1) | (SEQ ID NO: 38) | |
| 44 | topoisomerase | GUCUAAACCGACCCAA | CCCUUUUCGUACAC | Cas12a(2) |
| | | UAAUUUCUACUGUUG | ACGUAUCAGCGGGG | |
| | | UAGAU | U | |
| | | (SEQ ID NO: 1) | (SEQ ID NO: 39) | |
| 45 | topoisomerase | GUCUAAACCGACCCAA | UCAAAAUGUUUGAA | Cas12a(2) |
| | | UAAUUUCUACUGUUG | CAUGCUUUUUACUU | |
| | | UAGAU | C | |
| | | (SEQ ID NO: 1) | (SEQ ID NO: 40) | |
| 46 | helicase | GUCUAAACCGACCCAA | UCCGCGUUUAAAGG | Cas12a(2) |
| | | UAAUUUCUACUGUUG | GCUAUUCGUUGUUU | |
| | | UAGAU | U | |
| | | (SEQ ID NO: 1) | (SEQ ID NO: 41) | |
| 47 | helicase | GUCUAAACCGACCCAA | GGCAGCUGCUGAAC | Cas12a(2) |
| | | UAAUUUCUACUGUUG | AGCCUCUGAUAAUU | |
| | | UAGAU | U | |
| | | (SEQ ID NO: 1) | (SEQ ID NO: 42) | |
| 48 | helicase | GUCUAAACCGACCCAA | GGCAAACGUAAAUC | Cas12a(2) |
| | | UAAUUUCUACUGUUG | GCGUUCAAAGUUUU | |
| | | UAGAU | C | |
| | | (SEQ ID NO: 1) | (SEQ ID NO: 43) | |
| 49 | polymerase | GUUUUAGAGCUAGAA | GGUUCCCUUAACAG | Cas9 |
| | | AUAGCAAGUUAAAAU | GGCUAU | |
| | | AAGGCUAGUCCGUUA | (SEQ ID NO: 44) | |
| | | UCAACUUGAAAAAGU | | |
| | | GGCACCGAGUCGGUGC | | |
| | | (SEQ ID NO: 2) | | |
| 50 | polymerase | GUUUUAGAGCUAGAA | GUAGAUACAGUCAU | Cas9 |
| | | AUAGCAAGUUAAAAU | ACAACG | |
| | | AAGGCUAGUCCGUUA | (SEQ ID NO: 45) | |
| | | UCAACUUGAAAAAGU | | |
| | | GGCACCGAGUCGGUGC | | |
| | | (SEQ ID NO: 2) | | |
| 51 | polymerase | GUUUUAGAGCUAGAA | UUGUUUUUCCACGA | Cas9 |
| | | AUAGCAAGUUAAAAU | UAACGU | |
| | | AAGGCUAGUCCGUUA | (SEQ ID NO: 46) | |
| | | UCAACUUGAAAAAGU | | |
| | | GGCACCGAGUCGGUGC | | |
| | | (SEQ ID NO: 2) | | |
| 52 | topoisomerase | GUUUUAGAGCUAGAA | GCCGUCGGCGGUGG | Cas9 |
| | | AUAGCAAGUUAAAAU | UAAGAA | |
| | | AAGGCUAGUCCGUUA | (SEQ ID NO: 47) | |
| | | UCAACUUGAAAAAGU | | |
| | | GGCACCGAGUCGGUGC | | |
| | | (SEQ ID NO: 2) | | |
| 53 | topoisomerase | GUUUUAGAGCUAGAA | GCGCCAUCACAGAG | Cas9 |
| | | AUAGCAAGUUAAAAU | CCCGUG | |
| | | AAGGCUAGUCCGUUA | (SEQ ID NO: 48) | |
| | | UCAACUUGAAAAAGU | | |
| | | GGCACCGAGUCGGUGC | | |
| | | (SEQ ID NO: 2) | | |
| 54 | topoisomerase | GUUUUAGAGCUAGAA | UGUCACGUAAUGUU | Cas9 |
| | | AUAGCAAGUUAAAAU | GUUUUG | |
| | | AAGGCUAGUCCGUUA | (SEQ ID NO: 49) | |
| | | UCAACUUGAAAAAGU | | |
| | | GGCACCGAGUCGGUGC | | |
| | | (SEQ ID NO: 2) | | |
| 55 | helicase | GUUUUAGAGCUAGAA | GGCAAGCGACAGGC | Cas9 |
| | | AUAGCAAGUUAAAAU | UCAAAA | |
| | | AAGGCUAGUCCGUUA | (SEQ ID NO: 50) | |
| | | UCAACUUGAAAAAGU | | |
| | | GGCACCGAGUCGGUGC | | |
| | | (SEQ ID NO: 2) | | |
| 56 | helicase | GUUUUAGAGCUAGAA | CCUUCUUCCAAUAC | Cas9 |
| | | AUAGCAAGUUAAAAU | UUUUAG | |
| | | AAGGCUAGUCCGUUA | (SEQ ID NO: 51) | |
| | | UCAACUUGAAAAAGU | | |
| | | GGCACCGAGUCGGUGC | | |
| | | (SEQ ID NO: 2) | | |
| 57 | helicase | GUUUUAGAGCUAGAA | UGCACGGUUUUCUC | Cas9 |
| | | AUAGCAAGUUAAAAU | CUCGGG | |
| | | AAGGCUAGUCCGUUA | (SEQ ID NO: 52) | |
| | | UCAACUUGAAAAAGU | | |
| | | GGCACCGAGUCGGUGC | | |
| | | (SEQ ID NO: 2) | | |
| 58 | polymerase | GGCGCGUUUAUUCCA | GAGGAGCAUAGAAU | CasX |
| | | UUACUUUGGAGCCAG | GGUGGU | |
| | | UCCCAGCGACUAUGUC | (SEQ ID NO: 53) | |
| | | GUAUGGACGAAGCGC | | |
| | | UUAUUUAUCGGAGAG | | |
| | | AAACCGAUAAGUAAA | | |
| | | ACGCAUCAAAG | | |
| | | (SEQ ID NO: 4) | | |
| 59 | polymerase | GGCGCGUUUAUUCCA | CCCUUGGCCAUGAG | CasX |
| | | UUACUUUGGAGCCAG | GCUUUU | |
| | | UCCCAGCGACUAUGUC | (SEQ ID NO: 54) | |
| | | GUAUGGACGAAGCGC | | |
| | | UUAUUUAUCGGAGAG | | |
| | | AAACCGAUAAGUAAA | | |
| | | ACGCAUCAAAG | | |
| | | (SEQ ID NO: 4) | | |
| 60 | polymerase | GGCGCGUUUAUUCCA | AAACCGUUUUAACG | CasX |
| | | UUACUUUGGAGCCAG | AUUUCA | |
| | | UCCCAGCGACUAUGUC | (SEQ ID NO: 55) | |
| | | GUAUGGACGAAGCGC | | |
| | | UUAUUUAUCGGAGAG | | |
| | | AAACCGAUAAGUAAA | | |
| | | ACGCAUCAAAG | | |
| | | (SEQ ID NO: 4) | | |
| 61 | topoisomerase | GGCGCGUUUAUUCCA | GCCCGACGCCGUUG | CasX |
| | | UUACUUUGGAGCCAG | GUUCCC | |
| | | UCCCAGCGACUAUGUC | (SEQ ID NO: 56) | |
| | | GUAUGGACGAAGCGC | | |
| | | UUAUUUAUCGGAGAG | | |
| | | AAACCGAUAAGUAAA | | |
| | | ACGCAUCAAAG | | |
| | | (SEQ ID NO: 4) | | |
| 62 | topoisomerase | GGCGCGUUUAUUCCA | UGAUGACCCCUCCC | CasX |
| | | UUACUUUGGAGCCAG | AGGGAG | |
| | | UCCCAGCGACUAUGUC | (SEQ ID NO: 57) | |
| | | GUAUGGACGAAGCGC | | |
| | | UUAUUUAUCGGAGAG | | |
| | | AAACCGAUAAGUAAA | | |
| | | ACGCAUCAAAG | | |
| | | (SEQ ID NO: 4) | | |
| 63 | topoisomerase | GGCGCGUUUAUUCCA | AAUGGCCCUUGAAA | CasX |
| | | UUACUUUGGAGCCAG | UUGUAA | |
| | | UCCCAGCGACUAUGUC | (SEQ ID NO: 58) | |
| | | GUAUGGACGAAGCGC | | |
| | | UUAUUUAUCGGAGAG | | |
| | | AAACCGAUAAGUAAA | | |
| | | ACGCAUCAAAG | | |
| | | (SEQ ID NO: 4) | | |
| 64 | helicase | GGCGCGUUUAUUCCA | UCCUCCUUUACUGU | CasX |
| | | UUACUUUGGAGCCAG | UAGCAU | |
| | | UCCCAGCGACUAUGUC | (SEQ ID NO: 59) | |
| | | GUAUGGACGAAGCGC | | |
| | | UUAUUUAUCGGAGAG | | |
| | | AAACCGAUAAGUAAA | | |
| | | ACGCAUCAAAG | | |
| | | (SEQ ID NO: 4) | | |
| 65 | helicase | GGCGCGUUUAUUCCA | CCAUAAGAUCUUUC | CasX |
| | | UUACUUUGGAGCCAG | CCAUCC | |
| | | UCCCAGCGACUAUGUC | (SEQ ID NO: 60) | |
| | | GUAUGGACGAAGCGC | | |
| | | UUAUUUAUCGGAGAG | | |
| | | AAACCGAUAAGUAAA | | |
| | | ACGCAUCAAAG | | |
| | | (SEQ ID NO: 4) | | |
| 66 | helicase | GGCGCGUUUAUUCCA | CUUGAUCCUCUCGG | CasX |
| | | UUACUUUGGAGCCAG | GCCUUA | |
| | | UCCCAGCGACUAUGUC | (SEQ ID NO: 61) | |
| | | GUAUGGACGAAGCGC | | |
| | | UUAUUUAUCGGAGAG | | |
| | | AAACCGAUAAGUAAA | | |
| | | ACGCAUCAAAG | | |
| | | (SEQ ID NO: 4) | | |
| 67 | polymerase | CACCCGUGCAAAAUUG | UUUAAGGGCGGUUC | Cas13d |
| | | CAGGGGUCUAAAAC | GUUUGUUAAGUAG | |
| | | (SEQ ID NO: 3) | AU | |
| | | | (SEQ ID NO: 35) | |
| 68 | polymerase | CACCCGUGCAAAAUUG | UCCGGUCUCCACGC | Cas13d |
| | | CAGGGGUCUAAAAC | AUCUGUUUGGAUGA | |
| | | (SEQ ID NO: 3) | A | |
| | | | (SEQ ID NO: 36) | |
| 69 | polymerase | CACCCGUGCAAAAUUG | UUCAGCUUUAAUAG | Cas13d |
| | | CAGGGGUCUAAAAC | CAGUUCUACAUGAG | |
| | | (SEQ ID NO: 3) | U | |
| | | | (SEQ ID NO: 37) | |
| 70 | topoisomerase | CACCCGUGCAAAAUUG | GGGGACACGACCGC | Cas13d |
| | | CAGGGGUCUAAAAC | CGCAACCUGCAGAG | |
| | | (SEQ ID NO: 3) | G | |
| | | | (SEQ ID NO: 38) | |
| 71 | topoisomerase | CACCCGUGCAAAAUUG | CCCUUUUCGUACAC | Cas13d |
| | | CAGGGGUCUAAAAC | ACGUAUCAGCGGGG | |
| | | (SEQ ID NO: 3) | U | |
| | | | (SEQ ID NO: 39) | |
| 72 | topoisomerase | CACCCGUGCAAAAUUG | UCAAAAUGUUUGAA | Cas13d |
| | | CAGGGGUCUAAAAC | CAUGCUUUUUACUU | |
| | | (SEQ ID NO: 3) | C | |
| | | | (SEQ ID NO: 40) | |
| 73 | helicase | CACCCGUGCAAAAUUG | UCCGCGUUUAAAGG | Cas13d |
| | | CAGGGGUCUAAAAC | GCUAUUCGUUGUUU | |
| | | (SEQ ID NO: 3) | U | |
| | | | (SEQ ID NO: 41) | |
| 74 | helicase | CACCCGUGCAAAAUUG | GGCAGCUGCUGAAC | Cas13d |
| | | CAGGGGUCUAAAAC | AGCCUCUGAUAAUU | |
| | | (SEQ ID NO: 3) | U | |
| | | | (SEQ ID NO: 42) | |
| 75 | helicase | CACCCGUGCAAAAUUG | GGCAAACGUAAAUC | Cas13d |
| | | CAGGGGUCUAAAAC | GCGUUCAAAGUUUU | |
| | | (SEQ ID NO: 3) | C | |
| | | | (SEQ ID NO: 43) | |

In some embodiments, a nucleic acid sequence encodes a Guide RNA comprising: i) a repeat/tracrRNA, paired with ii) a Spacer RNA sequence corresponding to an ASFV Target gene. In some embodiments, the nucleic acid sequence is transcribed from a vector. In some embodiments, the vector further comprises a nucleic acid sequence encoding a Cas protein or functional fragment thereof. In some embodiments, the vector does not comprise a nucleic acid sequence encoding a Cas protein or functional fragment thereof. In some embodiments, the vector is administered simultaneously or sequentially with a second vector comprising a nucleic acid sequence encoding a Cas protein or functional fragment thereof. In some embodiments, the vector further comprises the nucleic acid sequence encoding the Cas protein or functional fragment thereof transcribes the Guide RNA corresponding to the sequence of the repeat/tracrRNA, paired with the Spacer RNA sequence corresponding to the ASFV Target gene. In some embodiments, a Cas protein or functional fragment thereof encloses the guide RNA and binds the structure with specific interactions in a number of domains. In some embodiments, the Guide RNA recognizes a target DNA or RNA region of interest corresponding to an ASFV target gene or ASFV target RNA and directs a Cas protein or functional fragment thereof to the ASFV target gene or ASFV target RNA. In some embodiments, the direction of the Cas protein or functional fragment thereof to the ASFV target gene or ASFV target RNA enables the directed Cas protein or functional fragment thereof to target endonuclease activity to a specific sequence of interest within the ASFV target gene or ASFV target RNA. In some embodiments, the targeted endonuclease activity to a specific sequence of interest within the ASFV target gene or ASFV target RNA inhibits ASFV viral replication. In some embodiments, the targeted endonuclease activity to a specific sequence of interest within the ASFV target gene or ASFV target RNA abrogates ASFV viral replication. In some embodiments, the targeted endonuclease activity to a specific sequence of interest within the ASFV target gene or ASFV target RNA diminishes ASFV viral replication. In some embodiments, the targeted endonuclease activity to a specific sequence of interest within the ASFV target gene or ASFV target RNA decreases an ASFV viral load. In some embodiments, the targeted endonuclease activity to a specific sequence of interest within the ASFV target gene or ASFV target RNA inhibits an exposure to ASFV in a subject following the administering from said exposure later developing into an acute ASFV infection. In some embodiments, the targeted endonuclease activity to a specific sequence of interest within the ASFV target gene or ASFV target RNA serves as an antiviral agent preventing or minimizing ASFV infection in the subject. In some embodiments, the targeted endonuclease activity to a specific sequence of interest within the ASFV target gene or ASFV target RNA serves as an antiviral agent, wherein the antiviral agent i) prevents ASFV infection, or ii) reduces an extent of ASFV infection in the subject.

In some embodiments, the Guide RNA sequence transcribed from the nucleic acid comprises a repeat/tracrRNA sequence paired with a Spacer RNA sequence. In some embodiments, the paired repeat/tracrRNA sequence and Spacer RNA sequence are listed in Table 2. In some embodiments, the Guide RNA associates with the Cas protein or functional fragment thereof to elicit site-directed Cas-mediated endonuclease activity to a specific ASFV Target DNA within one or a plurality of cells in a subject. In some embodiments, the Guide RNA sequence comprises a repeat/tracrRNA of SEQ ID NO: 1 paired with Spacer RNA sequence selected from SEQ ID NO: 5-7 that targets an ASFV Helicase DNA sequence when combined with Cas12a(2) protein. In some embodiments, the Guide RNA sequence comprises a repeat/tracrRNA of SEQ ID NO: 1 paired with Spacer RNA sequence selected from SEQ ID NO: 8-10 that targets an ASFV topoisomerase DNA sequence when combined with Cas12a(2) protein. In some embodiments, the Guide RNA sequence comprises a repeat/tracrRNA of SEQ ID NO: 1 paired with Spacer RNA sequence selected from SEQ ID NO: 11-13 that targets an ASFV Polymerase DNA sequence when combined with Cas12a(2) protein. In some embodiments, the Guide RNA sequence comprises a repeat/tracrRNA of SEQ ID NO: 2 paired with Spacer RNA sequence selected from SEQ ID NO: 14-16 that targets an ASFV Polymerase DNA sequence when combined with Cas9 protein. In some embodiments, the Guide RNA sequence comprises a repeat/tracrRNA of SEQ ID NO: 2 paired with Spacer RNA sequence selected from SEQ ID NO: 17-19 that targets an ASFV topoisomerase DNA sequence when combined with Cas9 protein. In some embodiments, the Guide RNA sequence comprises a repeat/tracrRNA of SEQ ID NO: 2 paired with Spacer RNA sequence selected from SEQ ID NO: 20-22 that targets an ASFV Helicase DNA sequence when combined with Cas9 protein. In some embodiments, the Guide RNA sequence comprises a repeat/tracrRNA of SEQ ID NO: 2 paired with Spacer RNA sequence corresponding to a scrambled RNA sequence selected from SEQ ID NO: 23-25 that when combined with Cas9 protein serves as negative control in experiments targeting ASFV DNA regions for site-specific Cas-mediated endonuclease targeting. In some embodiments, the Guide RNA sequence comprises a repeat/tracrRNA of SEQ ID NO: 3 paired with Spacer RNA sequence selected from SEQ ID NO: 5-7 that targets an ASFV Helicase DNA sequence when combined with Cas13d protein. In some embodiments, the Guide RNA sequence comprises a repeat/tracrRNA of SEQ ID NO: 3 paired with Spacer RNA sequence selected from SEQ ID NO: 8-10 that targets an ASFV topoisomerase DNA sequence when combined with Cas13d protein. In some embodiments, the Guide RNA sequence comprises a repeat/tracrRNA of SEQ ID NO: 3 paired with Spacer RNA sequence selected from SEQ ID NO: 11-13 that targets an ASFV Polymerase DNA sequence when combined with Cas13d protein. In some embodiments, the Guide RNA sequence comprises a repeat/tracrRNA of SEQ ID NO: 4 paired with Spacer RNA sequence selected from SEQ ID NO: 26-28 that targets an ASFV Helicase DNA sequence when combined with CasX protein. In some embodiments, the Guide RNA sequence comprises a repeat/tracrRNA of SEQ ID NO: 4 paired with Spacer RNA sequence selected from SEQ ID NO: 29-31 that targets an ASFV topoisomerase DNA sequence when combined with CasX protein. In some embodiments, the Guide RNA sequence comprises a repeat/tracrRNA of SEQ ID NO: 4 paired with Spacer RNA sequence selected from SEQ ID NO: 32-34 that targets an ASFV Polymerase DNA sequence when combined with CasX protein. In some embodiments, the Guide RNA sequence comprises a repeat/tracrRNA of SEQ ID NO: 1 paired with Spacer RNA sequence selected from SEQ ID NO: 35-37 that targets an ASFV Helicase DNA sequence when combined with Cas12a(2) protein. In some embodiments, the Guide RNA sequence comprises a repeat/tracrRNA of SEQ ID NO: 1 paired with Spacer RNA sequence selected from SEQ ID NO: 38-40 that targets an ASFV topoisomerase DNA sequence when combined with Cas12a(2) protein. In some embodiments, the Guide RNA sequence comprises a repeat/tracrRNA of SEQ ID NO: 1 paired with Spacer RNA sequence selected from SEQ ID NO: 41-43 that targets an ASFV Polymerase DNA sequence when combined with Cas12a(2) protein. In some embodiments, the Guide RNA sequence comprises a repeat/tracrRNA of SEQ ID NO: 2 paired with Spacer RNA sequence selected from SEQ ID NO: 44-46 that targets an ASFV Polymerase DNA sequence when combined with Cas9 protein. In some embodiments, the Guide RNA sequence comprises a repeat/tracrRNA of SEQ ID NO: 2 paired with Spacer RNA sequence selected from SEQ ID NO: 47-49 that targets an ASFV topoisomerase DNA sequence when combined with Cas9 protein. In some embodiments, the Guide RNA sequence comprises a repeat/tracrRNA of SEQ ID NO: 2 paired with Spacer RNA sequence selected from SEQ ID NO: 50-52 that targets an ASFV Helicase DNA sequence when combined with Cas9 protein. In some embodiments, the Guide RNA sequence comprises a repeat/tracrRNA of SEQ ID NO: 4 paired with Spacer RNA sequence selected from SEQ ID NO: 53-55 that targets an ASFV Polymerase DNA sequence when combined with CasX protein. In some embodiments, the Guide RNA sequence comprises a repeat/tracrRNA of SEQ ID NO: 4 paired with Spacer RNA sequence selected from SEQ ID NO: 56-58 that targets an ASFV topoisomerase DNA sequence when combined with CasX protein. In some embodiments, the Guide RNA sequence comprises a repeat/tracrRNA of SEQ ID NO: 4 paired with Spacer RNA sequence selected from SEQ ID NO: 59-61 that targets an ASFV Helicase DNA sequence when combined with CasX protein. In some embodiments, the Guide RNA sequence comprises a repeat/tracrRNA of SEQ ID NO: 3 paired with Spacer RNA sequence selected from SEQ ID NO: 35-37 that targets an ASFV Polymerase DNA sequence when combined with Cas13d protein. In some embodiments, the Guide RNA sequence comprises a repeat/tracrRNA of SEQ ID NO: 3 paired with Spacer RNA sequence selected from SEQ ID NO: 38-40 that targets an ASFV topoisomerase DNA sequence when combined with Cas13d protein. In some embodiments, the Guide RNA sequence comprises a repeat/tracrRNA of SEQ ID NO: 3 paired with Spacer RNA sequence selected from SEQ ID NO: 41-43 that targets an ASFV Helicase DNA sequence when combined with Cas13d protein.

In some embodiments, the Guide RNA sequence transcribed from the nucleic acid comprises a repeat/tracrRNA sequence paired with a Spacer RNA sequence. In some embodiments, the paired repeat/tracrRNA sequence and Spacer RNA sequence are listed in Table 2. In some embodiments, the Guide RNA associates with the Cas protein or functional fragment thereof to elicit site-directed Cas-mediated endonuclease activity specific ASFV Target RNA molecules within one or a plurality of cells in a subject. In some embodiments, the Guide RNA sequence comprises a repeat/tracrRNA of SEQ ID NO: 1 paired with Spacer RNA sequence selected from SEQ ID NO: 5-7 that targets an ASFV Helicase RNA sequence when combined with Cas12a(2) protein. In some embodiments, the Guide RNA sequence comprises a repeat/tracrRNA of SEQ ID NO: 1 paired with Spacer RNA sequence selected from SEQ ID NO: 8-10 that targets an ASFV topoisomerase RNA sequence when combined with Cas12a(2) protein. In some embodiments, the Guide RNA sequence comprises a repeat/tracrRNA of SEQ ID NO: 1 paired with Spacer RNA sequence selected from SEQ ID NO: 11-13 that targets an ASFV Polymerase RNA sequence when combined with Cas12a(2) protein. In some embodiments, the Guide RNA sequence comprises a repeat/tracrRNA of SEQ ID NO: 1 paired with Spacer RNA sequence selected from SEQ ID NO: 35-37 that targets an ASFV Helicase RNA sequence when combined with Cas12a(2) protein. In some embodiments, the Guide RNA sequence comprises a repeat/tracrRNA of SEQ ID NO: 1 paired with Spacer RNA sequence selected from SEQ ID NO: 38-40 that targets an ASFV topoisomerase RNA sequence when combined with Cas12a(2) protein. In some embodiments, the Guide RNA sequence comprises a repeat/tracrRNA of SEQ ID NO: 1 paired with Spacer RNA sequence selected from SEQ ID NO: 41-43 that targets an ASFV Polymerase RNA sequence when combined with Cas12a(2) protein.

In some embodiments, the Guide RNA sequence transcribed from the nucleic acid comprises a repeat/tracrRNA sequence paired with a Spacer RNA sequence. In some embodiments, the paired repeat/tracrRNA sequence and Spacer RNA sequence are listed in Table 2. In some embodiments, the specific i) repeat/tracrRNA sequence, paired with a specific ii) Spacer RNA sequence, to be used in CRISPR complex with a specific iii) Cas protein is listed in Table 2 as a Pairing. In some embodiments, the Pairing defining a composition described herein, which additionally may be used in a method described herein, is any combination of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75. In some embodiments, the Pairing defining a composition described herein, which additionally may be used in a method described herein, is at least nine of the following selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75. In some embodiments, the Pairing defining a composition described herein, which additionally may be used in a method described herein, is at least eight of the following selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75. In some embodiments, the Pairing defining a composition described herein, which additionally may be used in a method described herein, is at least seven of the following selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75. In some embodiments, the Pairing defining a composition described herein, which additionally may be used in a method described herein, is at least six of the following selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75. In some embodiments, the Pairing defining a composition described herein, which additionally may be used in a method described herein, is at least five of the following selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75. In some embodiments, the Pairing defining a composition described herein, which additionally may be used in a method described herein, is at least four of the following selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75. In some embodiments, the Pairing defining a composition described herein, which additionally may be used in a method described herein, is at least three of the following selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75. In some embodiments, the Pairing defining a composition described herein, which additionally may be used in a method described herein, is at least two of the following selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75. In some embodiments, the Pairing defining a composition described herein, which additionally may be used in a method described herein, is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75.

The disclosure relates to a composition comprising a cell with any one or combination of nucleic acid sequences disclosed herein. In some embodiments, the cell is a plant, insect or mammalian cell. In some embodiments, the cell is a eukaryotic cell or a prokaryotic cell, the cell may be isolated from the body of a mammal, a component of a culture system, or part of an organism, such as a pig or minipig. In some embodiments, the system and methods described herein include at least two components: (1) the RNAs or DNA/RNA hybrid (guide nucleic acid, a crRNA, tracrRNA, and/or a single cr/tracrRNA hybrid) targeted to a particular ASFV sequence in a cell; and (2) a Cas protein disclosed herein. In some cases, a system also can include a nucleic acid containing a donor sequence targeted to a sequence in the cell. The donor sequence and the guide sequence may be on one or a plurality of nucleic acid molecules. The Cas protein disclosed herein can create targeted DNA double-strand breaks at the desired viral nucleotide sequence (or loci), and the host cell can repair the double-strand break using the provide donor DNA sequence, thereby incorporating the modification stably into the viral DNA. In some embodiments, the viral DNA is cleaved and repaired after removing DNA between two or more cleavage events, such that the viral DNA is ligated with a significant deletion of genomic nucleic acid sequence. The resultant deletion can cause mutated or silenced viral genes and therefore disrupt viral expression and/or assembly.

Exemplary ASFV helicase protein sequence, ASFV topoisomerase protein sequence, ASFV DNA polymerase sequence, and an African Swine Fever Virus genomic DNA portion are listed in Table 3. In some embodiments, the guide sequence comprises a DNA target region complementary for an ASFV protein or portion of an ASFV genomic DNA sequence listed in Table 3. The disclosure relates to a composition comprising a guide sequence comprising a DNA target region complementary to a nucleic acid encoding from about 3 to about 30 amino acids from ASFV helicase. In some embodiments, the ASFV helicase comprises the amino acid sequence of SEQ ID NO: 62 listed in Table 3. In some embodiments, the guide sequence comprises a DNA target region complementary for an ASFV protein or portion of an ASFV genomic DNA sequence listed in Table 3. In some embodiments, compositions described herein comprise a guide sequence comprising a DNA target region complementary to a nucleic acid encoding from about 3 to about 30 amino acids from ASFV topoisomerase. In some embodiments, the ASFV topoisomerase comprises the amino acid sequence of SEQ ID NO: 63 listed in Table 3. The disclosure relates to a composition comprising a guide sequence comprising a DNA target region complementary to a nucleic acid encoding from about 3 to about 30 amino acids from ASFV polymerase. In some embodiments, the ASFV DNA polymerase comprises the amino acid sequence of SEQ ID NO: 64 listed in Table 3.

In some embodiments, the ASFV polymerase is: The disclosure relates to a composition comprising a guide sequence comprising a DNA target region complementary to a nucleic acid comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to from about 9 to about 90 nucleotide segment of the sequence of SEQ ID NO: 65 listed in Table 3. In some embodiments, the guide sequence comprising a DNA target region complementary to a nucleic acid comprises an RNA sequence complementary to a portion of between about 3 to about 30 nucleotides in length of SEQ ID NO: 65.

TABLE 3
Exemplary ASFV proteins and a portion of ASFV genomic DNA

| | ASFV | |
|---|---|---|
| SEQ ID | Protein or | |
| NO: | DNA | Sequence |
| 62 | ASFV helicase | MAYPELDAADFLQQLARRKEFKSLISPPVDQKELIRDLR |
| | protein | AHFVQIGGPGCEKGGRAFFPCDPYASPFPSIKGLQLHNA |
| | sequence | QLFVQNFQNPNTPYSRLLLNWQTGTGKSIAAIAIARQFM |
| | | NHYMNFIENAPWIFVVGFTRAIIQTEMLRRPELGFVSYK |
| | | EVAELHRLLHIAKQSGSTTSVESRHLNGFVSTLKRRLTD |
| | | RNRGGFFQFYGYKEFASKLFNITSKGEEKNFDVLSLFHR |
| | | SDEAEDTLNENDISQFVQKISEAETNGLIRVNQKIMEQLR |
| | | GGLLIADEIHNVYNIQERNNYGIALQYVLDAFPPHQAPR |
| | | AVFMSATPVTGSVMEYVDLLNLLVPRHELPNGQPLQRQ |
| | | QLFDSSGHSVKWKKDALALVERLSTGRVSFLLDTNTNF |
| | | YPERIFAGKMLSYKDETLPYLHFIECPMSEYQLETLKQL |
| | | GPDPKISSNAYSIYDMVFPNPKFSKQTEPKAYGLFNSTET |
| | | PTALSMASTDWLLENGVQIIEPSRRAPFNVSGSFLSLQPP |
| | | THISGLAFYSGKYTQMMKDILSIIRQGRGKILIYHNRVR |
| | | MSGVLILQEILQSNGILNEVSSPVGTTRCSICAAIRDEHTH |
| | | SDHQFIPVRFTILHSEIEPAVRERSLALFNASSNLEGHQLR |
| | | ILIGSKVIVEGLNFQAVRYEMIMSLPLDIPRLIQVFGRVV |
| | | RKNSHMELPPSERNVTIYLYVSTTPDGGPELAKYAQKLK |
| | | EYILIQEGDKALRKHAIDGFTNQIKIDKPMLESLPLSPSIT |
| | | PANVGATVLNTFEAYGYGEQEVKTISNIIISLFMARPVW |
| | | TYSELWKAVSTPKLIQGITIDNKLFSEDNFALALISLCYS |
| | | KNQCKELWIQNRLCTIMHVPAKPEHLYVAAVLNHKKEP |
| | | VLDIETYIRDFQLPAMHSIRITKYLEHSQTKEPFQVLYEK |
| | | FQKDFQDEPMEQVLIHYPASFHYTMLEALIIDNLAGMG |
| | | ALVEVYKKFFIAFSKKDIQPFPDIFKIISHVPGDDNTLVGY |
| | | ATEDSVRLITSREDKTWHEIPLYMLNINVKRKENDIVIGY |
| | | MESKGKALKFKIRPPIQVLKKNEITDIRMLNRGAVCETR |
| | | GREEQQKIADQLGISLNLTKISAIKLCLLIRNNLLQKEME |
| | | ARNQPNGMQDGIRWFYLFNDKMPSLVHTS |
| 63 | ASFV | MEAFEISDFKEHAKKKSMWAGALNKVTISGLMGVFTED |
| | topoisomerase | EDLMALPIHRDHCPALLKIFDELIVNATDHERACHSKTK |
| | protein | KVTYIKISFDKGVFSCENDGPGIPIAKHEQASLIAKRDVY |
| | sequence | VPEVASCFFLAGTNINKAKDCIKGGTNGVGLKLAMVHS |
| | | QWAILTTADGAQKYVQQINQRLDIIEPPTITPSREMFTRIE |
| | | LMPVYQELGYAEPLSETEQADLSAWIYLRACQCAAYVG |
| | | KGTTIYYNDKPCRTGSVMALAKMYTLLSAPNSTIHTATI |
| | | KADAKPYSLHPLQVAAVVSPKFKKFEHVSIINGVNCVK |
| | | GEHVTFLKKTINEMVIKKFQQTIKDKNRKTTLRDSCSNIF |
| | | VVIVGSIPGIEWTGQRKDELSIAENVFKTHYSIPSSFLTSM |
| | | TRSIVDILLQSISKKDNHKQVDVDKYTRARNAGGKRAQ |
| | | DCMLLAAEGDSALSLLRTGLTLGKSNPSGPSFDFCGMIS |
| | | LGGVIMNACKKVTNITTDSGETIMVRNEQLTNNKVLQGI |
| | | VQVLGLDFNCHYKTQEERAKLRYGCIVACVDQDLDGC |
| | | GKILGLLLAYFHLFWPQLIIHGFVKRLLTPLIRVYEKGKT |
| | | MPVEFYYEQEFDAWAKKQTSLVNHTVKYYKGLAAHDT |
| | | HEVKSMFKHFDNMVYTFTLDDSAKELFHIYFGGESELR |
| | | KRELCTGVVPLTETQTQSIHSVRRIPCSLHLQVDTKAYK |
| | | LDAIERQIPNFLDGMTRARRKILAGGVKCFASNNRERKV |
| | | FQFGGYVADHMFYHHGDMSLNTSIIKAAQYYPGSSHLY |
| | | PVFIGIGSFGSRHLGGKDAGSPRYISVQLASEFIKTMFPAE |
| | | DSWLLPYVFEDGQRAEPEYYVPVLPLAIMEYGANPSEG |
| | | WKYTTWARQLEDILALVRAYVDKDNPKHELLHYAIKH |
| | | KITILPLRPSNYNFKGHLKRFGQYYYSYGTYDISEQRNIIT |
| | | ITELPLRVPTVAYIESIKKSSNRMTFIEEIIDYSSSETIEILV |
| | | KLKPNSLNRIVEEFKETEEQDSIENFLRLRNCLHSHLNFV |
| | | KPKGGIIEFNSYYEILYAWLPYRRELYQKRLMREHAVLK |
| | | LRIIMETAIVRYINESAELNLSHYEDEKEASRILSEHGFPP |
| | | LNHTLIISPEFASIEELNQKALQGCYTYILSLQARELLIAA |
| | | KTRRVEKIKKMQARLDKVEQLLQESPFPGASVWLEEID |
| | | AVEKAIIKGRNTQWKFH |
| 64 | ASFV | MLTLIQGKKIVNDLRSRLAFEYNGQLIKILSKNIVAVGSL |
| | polymerase | RREEKMLNDVDLLIIVPEKKLLKHVLPNIRIKDLSFSVKV |
| | protein | CGERKCVLFIEWKKNTYQLDLFTALAEEKPYAVLHFTG |
| | sequence | PVSYLIRIRAALKKKNYKLNQYGLFKNQTLVPLKITTEK |
| | | ELIKELGFTYRIPKKRL |
| 65 | African swine | ATGTTAACGCTTATTCAAGGAAAAAAAATTGTAAATG |
| | fever virus, | ACTTACGCTCCCGACTTGCGTTTGAATATAATGGACA |
| | portion of | ACTTATAAAAATTTTATCAAAAAACATCGTTGCTGTTG |
| | genomic | GTAGCTTAAGACGCGAAGAGAAAATGCTTAATGACGT |
| | sequence | GGATCTTCTTATTATTGTTCCAGAAAAAAAACTTTTAA |
| | | AACACGTCCTGCCCAACATTCGCATAAAGGATCTTTC |
| | | TTTTTCTGTAAAAGTCTGCGGAGAACGAAAGTGTGTA |
| | | CTTTTTATTGAATGGAAAAAAAACACATATCAACTTG |
| | | ATCTTTTTACGGCCTTAGCCGAGGAAAAGCCATACGC |
| | | AGTACTTCATTTTACGGGTCCCGTTTCTTATTTAATAA |
| | | GAATTCGAGCTGCGTTAAAAAAAAAGAATTATAAGCT |
| | | AAATCAGTATGGATTATTTAAAAATCAAACTTTAGTA |
| | | CCTCTAAAAATCACTACTGAAAAAGAACTTATTAAAG |
| | | AATTAGGATTTACGTATCGCATACCTAAGAAACGTTT |
| | | ATA |

The construct(s) containing the guide RNA or RNA/DNA hybrid molecules, crRNA, tracrRNA, cr/tracrRNA hybrid, Cas protein disclosed herein coding sequence, and, where applicable, donor sequence, can be delivered to a cell using, for example, biolistic bombardment, electrostatic potential or through transformation permeability reagents (reagents known to increase the permeability of the cell wall or cell membrane). Alternatively, the system components can be delivered using Agrobacterium-mediated transformation, insect vectors, grafting, or DNA abrasion, according to methods that are standard in the art, including those described herein. In some embodiments, the system components can be delivered in a viral vector (e.g., a vector from a DNA virus such as, without limitation, geminivirus, AAV, adenovirus, lentiviral strains attenuated for human use, bean yellow dwarf virus, wheat dwarf virus, tomato leaf curl virus, maize streak virus, tobacco leaf curl virus, tomato golden mosaic virus, or Faba bean necrotic yellow virus, or a vector from an RNA virus such as, without limitation, a tobravirus (e.g., tobacco rattle virus, tobacco mosaic virus), potato virus X, or barley stripe mosaic virus. Typically, in the context of an endogenous CRISPR system, formation of a CRISPR complex (comprising a guide sequence hybridized to a target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. Cleavage of ASFV DNA renders it inoperable and susceptible to degradation within a host cell. Without wishing to be bound by theory, the tracr sequence, which may comprise or consist of all or a portion of a wild-type tracr sequence (e.g., about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operatively linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional (bind the Cas protein or functional fragment thereof). In some embodiments, the tracr sequence has at least 50%, 60%, 70%, 80%, 90%, 95% or 99% of sequence complementarity along the length of the tracr mate sequence when optimally aligned. In some embodiments, one or more vectors driving expression of one or more elements of a CRISPR system are introduced into a host cell such that the presence and/or expression of the elements of the CRISPR system direct formation of a CRISPR complex at one or more target sites. For example, a Cas enzyme, a guide sequence linked to a tracr-mate sequence, and a tracr sequence could each be operatively linked to separate regulatory elements on separate vectors. Alternatively, two or more of the elements expressed from the same or different regulatory elements may be combined in a single vector, with one or more additional vectors providing any components of the CRISPR system not included in the first vector. With at least some of the modifications contemplated by this disclosure, in some embodiments, the guide sequence or RNA or DNA sequences that form a CRISPR complex are at least partially synthetic. The CRISPR system elements that are combined in a single vector may be arranged in any suitable orientation, such as one element located 5′ with respect to (“upstream” of) or 3′ with respect to (“downstream” of) a second element. In some embodiments, the disclosure relates to a composition comprising a chemically synthesized guide sequence. In some embodiments, the chemically synthesized guide sequence is used in conjunction with a vector comprising a coding sequence that encodes a CRISPR enzyme, such as a type II Cas9 protein. In some embodiments, the chemically synthesized guide sequence is used in conjunction with one or more vectors, wherein each vector comprises a coding sequence that encodes a CRISPR enzyme, such as a type II Cas9 protein. The coding sequence of one element may be located on the same or opposite strand of the coding sequence of a second element, and oriented in the same or opposite direction. In some embodiments, a single promoter drives expression of a transcript encoding a repeat/tracrRNA. CRISPR enzyme and one or more additional (second, third, fourth, etc.) guide sequences, tracr mate sequence (optionally operatively linked to the guide sequence), and a tracr sequence embedded within one or more intron sequences (e.g., each in a different intron, two or more in at least one intron, or all in a single intron). In some embodiments, the CRISPR enzyme, one or more additional guide sequence, tracr mate sequence, and/or tracr sequence are each a component of different nucleic acid sequences. For instance, in the case of a tracr and tracr mate sequences and in some embodiments, the disclosure relates to a composition comprising at least a first and second nucleic acid sequence, wherein the first nucleic acid sequence comprises a tracr sequence and the second nucleic acid sequence comprises a tracr mate sequence, wherein the first nucleic acid sequence is at least partially complementary to the second nucleic acid sequence such that the first and second nucleic acid form a duplex and wherein the first nucleic acid and the second nucleic acid either individually or collectively comprise a DNA-targeting domain, a Cas protein binding domain, and a transcription terminator domain. In some embodiments, the CRISPR enzyme, one or more additional guide sequence, tracr mate sequence, and tracr sequence are operatively linked to and expressed from the same promoter. In some embodiments, the disclosure relates to compositions comprising any one or combination of the disclosed domains on one guide sequence or two separate tracrRNA/crRNA sequences with or without any of the disclosed modifications. Any methods disclosed herein also relate to the use of tracrRNA/crRNA sequence interchangeably with the use of a guide sequence, such that a composition may comprise a single synthetic guide sequence and/or a synthetic tracrRNA/crRNA with any one or combination of modified domains disclosed herein. One or a plurality of vectors may also be components in any system or composition provided herein. In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operatively linked to the tracr mate sequence, such that following insertion of a guide sequence into the insertion site and upon expression, the guide sequence directs sequence-specific binding of a CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple, different guide sequences are used, a single expression construct may be used to target CRISPR activity to multiple, different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell. The disclosure relates to any composition comprising any of the aforementioned elements and one or more artificially synthesized guide sgRNA described herein. Another aspect of the disclosure relates to a CRISPR system comprising a modified CRISPR enzyme (or “Cas protein”) or a nucleotide sequence encoding one or more Cas proteins. Any protein capable of enzymatic activity in cooperation with a guide sequence is a Cas protein. In some embodiments, the disclosure relates to a system comprising a vector comprising a regulatory element operatively linked to an enzyme-coding sequence encoding a CRISPR enzyme, such as a Cas protein from the Cas family of enzymes.

In some embodiments, the disclosure relates to a system, composition, or pharmaceutical composition comprising any one or plurality of Cas proteins either individually or in combination with one or a plurality of guide sequences. Compositions of one or a plurality of Cas proteins may be administered to a subject with any of the disclosed guide sequences sequentially or contemporaneously. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, type V CRISPR-Cas systems, variants and fragments thereof, or modified versions thereof having at least 70% homology to the sequences of Table X, wherein are incorporated by reference in their entireties. These enzymes are known; for example, the amino acid sequence of S. pyogenes Cas9 protein may be found in the SwissProt database under accession number Q99ZW2.After an organism is infected with ASFV, the organism can be administered or transfected with a sequence encoding a Cas protein disclosed herein or a functional fragment thereof, a crRNA, a trRNA, a crRNA and a tracrRNA, a cr/tracrRNA hybrid, and/or a synthetic guide nucleic acid (and, in some cases, a donor sequence), any suitable method can be used to determine whether targeted mutagenesis has occurred at the target site. In some embodiments, a phenotypic change can indicate that a donor sequence has been integrated into the target site. PCR-based methods also can be used to ascertain whether a genomic target site contains targeted mutations or donor sequence, and/or whether precise recombination has occurred at the 5′ and 3′ ends of the donor. In some embodiments, a vector encodes a CRISPR enzyme comprising one or more nuclear localization signals (NLSs), such as about (or more than about) 1, 2, 3, 4, 5, 6, 7, 8, 9, 10, or more NLSs. In some embodiments, the CRISPR enzyme comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In a preferred embodiment of the disclosure, the CRISPR enzyme comprises at most 6 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Typically, an NLS consists of one or more short sequences of positively charged lysines or arginines exposed on the protein surface, but other types of NLS are known. Non-limiting examples of NLS s include an NLS sequence derived from: the NLS of the SV40 virus large T-antigen, having the amino acid sequence. In some embodiments, the CRISPR enzyme or Cas protein (used interchangeably) is free of a nuclear localization signal. In some embodiments, any domain comprises hybrid RNA/DNA sequences of either unmodified or modified nucleotides. In some embodiments, the DNA-targeting domain comprises no less than about 250, 200, 150, 100, 50, 45, 40, 35, 30, 25, or 20 nucleotides, wherein no more than about 50, 45, 40, 35, 30, 25, 20, 15, 14, 13, 12, 11, 10, 9, 8, 7, 6, 5, 4, 3, 2, or 1 nucleotides is a modified or unmodified deoxyribonucleic acid. In some embodiments, the DNA-targeting domain comprises no less than about 250, 200, 150, 100, 50, 45, 40, 35, 30, 25, or 20 nucleotides, wherein no more than about 50, 45, 40, 35, 30, 25, 20, 15, 14, 13, 12, 11, 10, 9, 8, 7, 6, 5, 4, 3, 2, or 1 nucleotides from the 5′ end of the guide sequence is a modified or unmodified deoxyribonucleic acid. In some embodiments, the Cas-binding domain comprises no less than about 250, 200, 150, 100, 50, 45, 40, 35, 30, 25, or 20 nucleotides, wherein no more than about 50, 45, 40, 35, 30, 25, 20, 15, 14, 13, 12, 11, 10, 9, 8, 7, 6, 5, 4, 3, 2, or 1 nucleotides is a modified or unmodified deoxyribonucleic acid. In some embodiments, the transcription terminator domain comprises no less than about 250, 200, 150, 100, 50, 45, 40, 35, 30, 25, or 20 nucleotides, wherein no more than about 50, 45, 40, 35, 30, 25, 20, 15, 14, 13 12, 11, 10, 9, 8, 7, 6, 5, 4, 3, 2, or 1 nucleotides is a modified or unmodified deoxyribonucleic acid. In some embodiments, the transcription terminator domain is free of modified or unmodified deoxyribonucleic acid. In some embodiments, the Cas-binding domain is free of modified or unmodified deoxyribonucleic acid. TABLE 1 Accession Numbers of Cas proteins (or those related with Cas-like function) and Nucleic Acids encoding the same. All amino acid and nucleic acid sequences associated with the Accession Numbers below as of Jul. 25, 2022, are incorporated by reference in their entireties. Any mutants or variants that comprise at least about 70, 75, 80, 85, 90, 95, 96, 97, 98, 99% sequence identity to the encoded nucleic acids or acids set forth in the Accession Numbers below are also incorporated by reference in their entireties: NC_014644.1; NC_002967.9, NC_007929.1; NC_000913.3 NC 004547.2, NC_009380.1; NC_011661.1; NC_010175.1; NC_010175.1; NC_010175.1; NC 003413.1; NC_000917.1; NC_002939.5; NC_018227.2; NC 004829.2, NC 021921.1; NC_014160.1; NC_011766.1; NC 007681.1; NC 021592.1; NC_021592.1; NC_021169.1; NC_020517.1; NC_018656.1; NC_018015.1; NC_018015.1; NC_017946.1; NC_017576.1; NC_017576.1; NC_015865.1; NC_015865.1; NC_015680.1; NC_015680.1; NC_015474.1; NC 015435.1; NC_013790.1; NC_013790.1; NC_012883.1; NC 012470.1; NC 016051.1; NC 010610.1; NC 009515.1; NC 008942.1; NC 007181.1; NC_007181.1; NC_006624.1; NC 006448.1; NC_002935.2; NC 002935.2; NC_002950.2, NC 002950.2, NC_002663.1; NC 002663.1; NC_004557.1; NC_004557.1; NC_019943.1; NC 019943.1; NC_019943.1; NC_017459.1; NC_017459.1; NC_015518.1; NC_015460.1; NC 015416.1; NC 014933.1; NC 013961.1; NC 013202.1; NC 013158.1; NC 009464.1; NC 008508.1; NC_007426.1; NC 000917.1; NC_003901.1; NC_003901.1; NC_003106.2, NC_009434.1; NC_005085.1; NC 005085.1; NC_020247.1; NC_020247.1; NC_020246.1; NC_020246.1; NC_018224.1; NC_015943.1; NC_011138.3; NC_009778.1; NC_006834.1; NC 014228.1; NC 010002.1; NC 013892.1; NC 010296.1; NC 009615.1; NC 012632.1; NC_012632.1; NC_012588.1; NC_012588.1; NC_007643.1; NC_002939.5; NC_011296.1; NC_011296.1; NC_018609.1; NC 021355.1; NC_021355.1; NC_020800.1; NC_019942.1; NC_019792.1; NC_015958.1; NC_015678.1; NC_015636.1; NC_015562.1; NC_014222.1; NC 014222.1; NC 014002.1; NC 013887.1; NC 013156.1; NC 011832.1; NC 009953.1; NC 009635.1; NC_009634.1; NC 008618.1; NC_007955.1; NC_007955.1; NC_007955.1; NC 007955.1; NC_007955.1; NC_007796.1; NC_002754.1; NC_002754.1; NC_011835.1; NC_013198.1; NC_000962.3; NC 002163.1; NC_017034.1; NC_009089.1; NC_008698.1; NC 020419.1; NC 020419.1; NC 020419.1; NC 015847.1; NC 014374.1; NC 013520.1; NC_010482.1; NC_009776.1; NC_009776.1; NC_009033.1; NC_000916.1; NC_018015.1; NC_015518.1; NC_014537.1; NC_009440.1; NC_007644.1; NC_007644.1; NC_022246.1; NC 019943.1; NC_016023.1; NC 016023.1; NC_015416.1; NC_013722.1; NC_013722.1; NC 009464.1; NC 007643.1; NC 007643.1; NC 007643.1; NC 003106.2; NC 004342.2; NC_018658.1; NC_017276.1; NC 017275.1; NC_016112.1; NC_016112.1; NC_003552.1; NC 003197.1; NC 003198.1; NC 012726.1; NC 012623.1; NC 015964.1; NC 023069.1; NC_023044.1; NC_022777.1; NC_022777.1; NC_022777.1; NC_013769.1; NC_013769.1; NC_011832.1; NC_011296.1; NC_009712.1; NC_009634.1; NC_009439.1; NC_009135.1; NC 008599.1; NC_007796.1; NC 007796.1; NC_007796.1; NC_007355.1; NC_021082.1; NC_018001.1; NC 009785 1; NC 022084.1; NC_018092.1; NC 014804.1; NC 014147.1; NC 009053.1; NC_000961.1; NC_000961.1; NC_021058.1; NC_018876.1; NC_018876.1; NC_018081.1; NC_011567.1; NC_016901.1; NC_014500.1; NC_013715.1; NC_019977.1; NC_019042.1; NC_017274.1; NC_015954.1; NC_015676.1; NC_015320.1; NC_014122.1; NC 014122.1; NC_013407.1; NC 014961.1; NC 013926.1; NC_013926.1; NC 021353.1; NC_008818.1; NC_021058.1; NC_015151.1; NC_013849.1; NC_009051.1; NC_018876.1; NC_018876.1; NC_014507.1; NC_015574.1; NC_014500.1; NC_012622.1; NC_012589.1; NC 009515.1; NC_017275.1; NC_000913.3; NC_017527.1; NC_018227.2; NC_007355.1; NC 014106.1; NC 010610.1; NC 008054.1; NC 007164.1; NC 015760.1; NC 009953.1; NC_010572.1; NC_009613.3; NC_014334.1; NC_008526.1; NC_026150.1; NC_015776.1; NC_007116.6; NC_012779 2; NC_003901.1; NC_020892.1; NC_011832.1; NC_003143.1; NC 003143.1; NC_008800.1; NC_011308.1; NC_008942.1; NC_007297.1; NC_005877.1; NC 005877.1; NC 002689 2; NC 006085.1; NC 004116 1; NC 010397.1; NC 009917.1; NC_012490.1; NC_006067.1; NW_004197518.1; NC_022777.1; NC_019042.1; NC_004547.2; NC_002695.1; NT_078267.5; NC_002656.1; NC_022774.1; NC_01109 1.1; NC_005881.2; NC_011183.1; NC_015937.1; NC_008584.1; NC_024122.1; NC_022768.1; NC 022772.1; NC_013085.1; NC_010154.1; NC 010152.1; NC_010155.1; NC 009804.1; NC 009803.1; NC_005342.2; NC_004333 2; NC_023735.1; NC_023694.1; NC_027364.1; NC_019526.1; NC_023607.1; NC 021353.1; NC_021592.1; NC_012039.1; NC_008942.1; NC_002936.3; NC_005877.1; NC_021169.1; NC_021058.1; NC_020517.1; NC_020388.1; NC 020388.1; NC_018656.1; NC_015435.1; NC 014804.1; NC_013790.1; NC_013790.1; NC 009440.1; NC_009051.1; NC 007929.1; NC_007929.1; NC_005042.1; NC_003454.1; NC_003238 2; NC_021313.1; NC_019943.1; NC_019943.1; NC_017459.1; NC_017384.1; NC_015288.1; NC_015287.1; NC_015284.1; NC_015281.1; NC_015280.1; NC_014334.1; NC_014297.1; NC_013967.1; NC_013202.1; NC_011129.1; NC 007426.1; NC 007426.1; NC_003901.1; NC_003901.1; NC_004342 2; NC_014622 2; NC_023731.1; NC_023729.1; NC_023716.1; NC_017275.1; NC_015574.1; NC_015216.1; NC_015216.1; NC_013922.1; NC_013922.1; NC_013743.1; NC_0129661; NC_012966.1; NC_011913.1; NC_010397.1; NC 010296.1; NC 009380.1; NC 006396.1; NC 006347.1; NC 002944 2; NC 003552.1; NC_004663.1; NW_006890135.1; NW_005819424.1; NW_005395962.1; NC_022273.1; NC 019466.1; NC 018739.2; NC 016132.1; NC 012593.1; NC 026744.1; NC 026585.1; NC_026584.1; NC_022067.1; NC_017274.1; NC_017274.1; NC_017274.1; NC_016563.1; NC_015562.1; NC_013769.1; NC_013769.1; NC_010175.1; NC_002754.1; NC_002754.1; NC 009089.1; NC_014374.1; NC 009776.1; NC_005877.1; NC_005877.1; NC_005877.1; NC 002689.2; NC 002689.2; NC 002689.2; NC 000918.1; NC 022093.1; NC 022093.1; NC 022093.1; NC_018092.1; NC_015931.1; NC_015931.1; NC_015931.1; NC_015865.1; NC_010482.1; NC_010482.1; NC_000916.1; NC_000961.1; NC_000961.1; NC_000853.1; NC 000853.1; NC_021313.1; NC_020388.1; NC_018876.1; NC_015151.1; NC_013849.1; NC 009440.1; NC 007426.1; NC 007181.1; NC 007181.1; NC 007181.1; NC 003106.2; NC 027207.1; NC_027206.1; NC 020247.1; NC_020247.1; NC_020247.1; NC_020246.1; NC_020246.1; NC_020246.1; NC_006347.1; NC_005140.1; NC_013486.1; NC_013486.1; NC_012726.1; NC_012632.1; NC_012589.1; NC_012588.1; NC_006038.1; and NC_012012.3.

### Methods:

In some embodiments, compounds and compositions described herein are useful in treating a, ASFV infection or symptoms associated with ASFV infection in a cell, symptoms associated with ASFV infection in a subject. Thus, provided herein are methods for treating an ASFV infection, comprising administering to a subject in need thereof, a therapeutically effective amount of a composition described herein or a pharmaceutically acceptable salt thereof, or a composition comprising a disclosed compound or pharmaceutically acceptable salt thereof. In some embodiments, the methods comprise a step of administering a composition comprising disclosed nucleic acid sequences that comprise a DNA targeting domain specific for an ASFV protein, such as an enzyme. Disorders treatable by the present compounds and compositions comprise fever, malaise, decrease in appetite, weakness, red blotchy skin, skin lesions, diarrhea, vomiting, coughing, difficulty breathing, spontaneous abortion, and sudden death. In some embodiments, the disclosure relates to any of the above disclosed methods disclosed herein, wherein the administrating step comprises administering a pharmaceutical composition comprising: (i) a pharmaceutically effective amount of any of the disclosed compounds; and (ii) a pharmaceutically acceptable carrier. Thus, in various embodiments, disclosed are methods for treating ASFV infection in a subject in need thereof, the method comprising administering to the subject a therapeutically effective amount of the Cas protein and/or guide or tracer RNA disclosed herein and a therapeutically effective amount of a compound selected from carprofen, mefenamic acid, phenacetin, valdecoxib, fenoldopam mesylate, Feverphenazine hydrochloride, bupivacaine HCl, phenazopyridine HCl, alverine citrate, nitenpyram, 4-aminobutyric acid (GABA), PF-3845, esmolol HCl, cimetidine, conivaptan HCl, (+)-MK-801 maleate, MK-0752, R04929097, rosmarinic acid, theophylline, aripiprazole, flopropione, latrepirdine 2HCl, ADX-47273, MPEP, nefopam HCl, phenazopyridine HCl, epinephrine, (+)-matrine, phenothiazine, naproxen sodium, AMG-517, isoliquiritigenin, nilvadipine, prednisone, and simvastatin, or a pharmaceutically acceptable salt thereof. In a further embodiment, the compound is selected from aripiprazole, dexmedetomidine, matrine, and MPEP, or a pharmaceutically acceptable salt thereof. In further embodiments, the compound is FDA approved. In further embodiments, the administering is accomplished by oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or combinations thereof. In various embodiments, the method further comprises administering an effective amount of an agent associated with the treatment of an ASFV infection in addition to the one or plurality of nucleic acid sequences and Cas proteins disclosed herein. Thus, in various embodiments, the method further comprises administering an agent known for the treatment of an ASFV infection. In some embodiments, the compound and the agent are administered simultaneously. In some embodiments, the compound and the agent are administered sequentially. In some embodiments, the compound and the agent are co-packaged. In some embodiments, the compound and the agent are co-formulated. The disclosure also relates to a method of altering expression of at least one ASFV gene or protein in a cell comprising introducing into a cell an engineered, non-naturally occurring CRISPR associated (Cas) (CRISPR-Cas) system comprising: (a) a vector comprising a nucleotide sequence encoding any CRISPR enzyme disclosed herein, any mutated CRISPR enzyme having at least 70%, 75%, 80%, 85%, 90%, 91%, 92%, 93%, 94%, 95%, 9%, 97%, 98%, or 99% sequence identity to any CRISPR enzyme disclosed herein, or functional fragment thereof; and (b) a nucleic acid sequence disclosed herein, wherein components (a) and (b) are located on same or different vectors of the system; wherein the cell contains and/or expresses an ASFV DNA molecule having a target sequence and encoding the gene product; and wherein the guide RNA targets and hybridizes with a DNA target sequence, the CRISPR enzyme or functional fragment thereof cleaves the DNA molecule, whereby expression of the at least one ASFV gene or protein is altered. In some embodiments, the ASFV gene is silenced by enzymatic cleavage of the ASFV genomic or endogenous DNA by the CRISPR-Cas system present in the cell after administration or exposure. The disclosure also relates to a method of altering expression of at least one viral gene product in a cell comprising introducing into a cell an engineered, non-naturally occurring CRISPR associated (Cas) (CRISPR-Cas) system comprising: (a) a vector comprising a nucleotide sequence encoding a Type I, Type-II, or Type III Cas9 protein or functional fragment thereof; and (b) a nucleic acid sequence disclosed herein, wherein components (a) and (b) are located on same or different vectors of the system; wherein the cell contains and expresses a DNA molecule having an ASFV target sequence and encoding the gene product; and wherein the guide RNA targets and hybridizes with a DNA target sequence and the Cas protein or functional fragment thereof cleaves the DNA molecule, whereby expression of the at least one gene product is altered. In some embodiments, the Cas protein is a Cas9, Cas12, Cas13, CasX, Cas12a2, or Cas13(d) protein, or a functional fragment or variant thereof. In some embodiments, Cas12a2 is a multi-turnover enzyme that degrades double stranded DNA, single-stranded DNA and single-stranded RNA. In some embodiments, Cas12a2 enzyme from Sulfuricurvum sp. is activated when its CRISPR RNA guide base pairs with an RNA target. In some embodiments, Cas12a2 indiscriminately degrades double stranded DNA, single-stranded DNA and single-stranded RNA once activated. In some embodiments, Cas12a2 indiscriminately degrades targeted double stranded DNA, targeted single-stranded DNA and targeted single-stranded RNA once activated. In some embodiments, like Cas12a2, Cas13 is a multi-turnover enzyme. In some embodiments, Cas 13 only targets RNA instead of DNA. In some embodiments, Cas9 or Cas12 function as single turnover endonucleases. In some embodiments, the composition or pharmaceutical composition comprises a nucleic acid molecule encoding a nucleic acid sequence encoding one or a plurality of Cas proteins chosen from: a Cas9, Cas12, Cas13, CasX, Cas12a2, or Cas13(d) protein, or a functional fragment or variant thereof. In some embodiments, methods of the disclosure comprise administering or exposing a cell or animal to the compositions or pharmaceutical compositions disclosed herein comprising: (i) a combination of Cas proteins chosen from: a Cas9, Cas12, Cas13, CasX, Cas12a2, or Cas13(d) protein, or a functional fragment or variant thereof; or (ii) a plurality of nucleic acid molecule comprising a nucleic acid sequence encoding one or a combination of Cas proteins chosen from: a Cas9, Cas12, Cas13, CasX, Cas12a2, or Cas13(d), or a functional fragment or variant thereof. The disclosure also relates to a method of treating or preventing growth and/or proliferation of ASFV in a subject diagnosed with or suspected of having ASFV infection, the method comprising administering to a subject diagnosed with ASFV or suspected of having ASFV one or more pharmaceutical compositions disclosed herein.

In some embodiments, the method for treating African Swine Fever in a subject in need thereof comprising administering to the subject a therapeutically effective amount of a first nucleic acid decreases an ASFV viral load in the subject. In some embodiments, the viral load is decreased by at least about 5%, 6%, 7%, 8%, 9%, 10%, 11%, 12%, 13%, 14%, 15%, 16%, 18%, 20%, 22%, 24%, 25%, 27%, 29%, 30%, 32%, 35%, 37%, 40%, 42%, 45%, 47%, 50%, 52%, 55%, 57%, 59%, 60%, 62%, 65%, 67%, 70%, 72%, 75%, 77%, 80%, 85%, 90%, 95%, 96%, 97%, 98%, or 99%, less than three weeks after the administering. In some embodiments, the viral load is decreased by at least about 5%, 6%, 7%, 8%, 9%, 10%, 11%, 12%, 13%, 14%, 15%, 16%, 18%, 20%, 22%, 24%, 25%, 27%, 29%, 30%, 32%, 35%, 37%, 40%, 42%, 45%, 47%, 50%, 52%, 55%, 57%, 59%, 60%, 62%, 65%, 67%, 70%, 72%, 75%, 77%, 80%, 85%, 90%, 95%, 96%, 97%, 98%, or 99%, less than two weeks after the administering. In some embodiments, the viral load is decreased by at least about 5%, 6%, 7%, 8%, 9%, 10%, 11%, 12%, 13%, 14%, 15%, 16%, 18%, 20%, 22%, 24%, 25%, 27%, 29%, 30%, 32%, 35%, 37%, 40%, 42%, 45%, 47%, 50%, 52%, 55%, 57%, 59%, 60%, 62%, 65%, 67%, 70%, 72%, 75%, 77%, 80%, 85%, 90%, 95%, 96%, 97%, 98%, or 99%, less than one week after the administering. In some embodiments, the viral load is decreased by at least about 5%, 6%, 7%, 8%, 9%, 10%, 11%, 12%, 13%, 14%, 15%, 16%, 18%, 20%, 22%, 24%, 25%, 27%, 29%, 30%, 32%, 35%, 37%, 40%, 42%, 45%, 47%, 50%, 52%, 55%, 57%, 59%, 60%, 62%, 65%, 67%, 70%, 72%, 75%, 77%, 80%, 85%, 90%, 95%, 96%, 97%, 98%, or 99%, less than five days after the administering. In some embodiments, the viral load is decreased by at least about 5%, 6%, 7%, 8%, 9%, 10%, 11%, 12%, 13%, 14%, 15%, 16%, 18%, 20%, 22%, 24%, 25%, 27%, 29%, 30%, 32%, 35%, 37%, 40%, 42%, 45%, 47%, 50%, 52%, 55%, 57%, 59%, 60%, 62%, 65%, 67%, 70%, 72%, 75%, 77%, 80%, 85%, 90%, 95%, 96%, 97%, 98%, or 99%, less than four days after the administering. In some embodiments, the viral load is decreased by at least about 5%, 6%, 7%, 8%, 9%, 10%, 11%, 12%, 13%, 14%, %, 16%, 18%, 20%, 22%, 24%, 25%, 27%, 29%, 30%, 32%, 35%, 37%, 40%, 42%, 45%, 47%, 50%, 52%, 55%, 57%, 59%, 60%, 62%, 65%, 67%, 70%, 72%, 75%, 77%, 80%, 85%, 90%, 95%, 96%, 97%, 98%, or 99%, less than three days after the administering. In some embodiments, the viral load is decreased by at least about 5%, 6%, 7%, 8%, 9%, 10%, 11%, 12%, 13%, 14%, 15%, 16%, 18%, 20%, 22%, 24%, 25%, 27%, 29%, 30%, 32%, 35%, 37%, 40%, 42%, 45%, 47%, 50%, 52%, 55%, 57%, 59%, 60%, 62%, 65%, 67%, 70%, 72%, 75%, 77%, 80%, 85%, 90%, 95%, 96%, 97%, 98%, or 99%, less than two days after the administering. In some embodiments, the viral load in the subject is decreased by at least about 10% less than one week after the administering. In some embodiments, the viral load in the subject is decreased by at least about 15% less than one week after the administering. In some embodiments, the viral load in the subject is decreased by at least about 20% less than one week after the administering. In some embodiments, the viral load in the subject is decreased by at least about 25% less than one week after the administering. In some embodiments, the viral load in the subject is decreased by at least about 30% less than one week after the administering. In some embodiments, the viral load in the subject is decreased by at least about 40% less than one week after the administering. In some embodiments, the viral load in the subject is decreased by at least about 50% less than one week after the administering. In some embodiments, the viral load in the subject is decreased by at least about 55% less than one week after the administering. In some embodiments, the viral load in the subject is decreased by at least about 60% less than one week after the administering. In some embodiments, the viral load in the subject is decreased by at least about 62% less than one week after the administering. In some embodiments, the viral load in the subject is decreased by at least about 64% less than one week after the administering. In some embodiments, the viral load in the subject is decreased by at least about 65% less than one week after the administering. In some embodiments, the viral load in the subject is decreased by at least about 66% less than one week after the administering. In some embodiments, the viral load in the subject is decreased by at least about 67% less than one week after the administering. In some embodiments, the viral load in the subject is decreased by at least about 68% less than one week after the administering. In some embodiments, the viral load in the subject is decreased by at least about 69% less than one week after the administering. In some embodiments, the viral load in the subject is decreased by at least about 70% less than one week after the administering. In some embodiments, the viral load in the subject is decreased by at least about 75% less than one week after the administering. In some embodiments, the viral load in the subject is decreased by at least about 80% less than one week after the administering. In some embodiments, the viral load in the subject is decreased by at least about 85% less than one week after the administering. In some embodiments, the viral load in the subject is decreased by at least about 90% less than one week after the administering. In some embodiments, the viral load in the subject is decreased by at least about 95% less than one week after the administering. In some embodiments, the viral load in the subject is decreased by at least about 97% less than one week after the administering. In some embodiments, the viral load in the subject is decreased by at least about 99% less than one week after the administering. In some embodiments, the first nucleic acid comprises: i) a first nucleic acid sequence that comprises at least about 70% complementary to an endogenous African Swine Fever Virus (ASFV) nucleic acid sequence encoding an ASFV helicase, an ASFV DNA polymerase, or an ASFV topoisomerase; and ii) a second nucleic acid sequence encoding a Cas endonuclease protein or functional fragment thereof. In some embodiments, wherein the second nucleic acid sequence encodes a multi-turnover Cas endonuclease, the viral load in the subject is decreased by at least about 67% less than one week after the administering. In some embodiments, wherein the second nucleic acid sequence encodes a multi-turnover Cas12a2 endonuclease, the viral load in the subject is decreased by at least about 67% less than one week after the administering. In some embodiments, wherein the second nucleic acid sequence encodes a multi-turnover Cas13b endonuclease, the viral load in the subject is decreased by at least about 5%, 6%, 7%, 8%, 9%, 10%, 11%, 12%, 13%, 14%, 15%, 16%, 18%, 20%, 22%, 24%, 25%, 27%, 29%, 30%, 32%, 35%, 37%, 40%, 42%, 45%, 47%, 50%, 52%, 55%, 57%, 59%, 60%, 62%, 65%, 67%, 70%, 72%, 75%, 77%, 80%, 85%, 90%, 95%, 96%, 97%, 98%, or 99%, less than one week after the administering.

According to one aspect, the disclosure relates to a method of altering a eukaryotic cell comprising: transfecting the eukaryotic cell with a nucleic acid disclosed herein complementary to genomic DNA of the ASFV viral genome, transfecting the eukaryotic cell with a nucleic acid encoding an enzyme that interacts with the RNA and cleaves the genomic DNA in a site-specific manner, wherein the cell expresses or comprises the RNA and the enzyme, the RNA binds to complementary genomic viral DNA and the enzyme cleaves the viral DNA in a site-specific manner. According to one aspect, the enzyme is Cas9 or modified Cas9 or a homolog of Cas9. According to one aspect, the enzyme is Cas12a or modified Cas12a or a homolog of Cas12a. According to one aspect, the enzyme is Cas12a2 or modified Cas12a2 or a homolog of Cas12a2. According to one aspect, the enzyme is Cas13b or modified Cas13b or a homolog of Cas13b. According to one aspect, the enzyme is CasX or modified CasX or a homolog of CasX. According to one aspect, the eukaryotic cell is a yeast cell, a plant cell or a mammalian cell. In some embodiments, the mammalian cell is a human cell. In some embodiments, the mammalian cell is a porcine cell. According to one aspect, the nucleic acid disclosed herein comprises from about 10 to about 250 nucleotides. According to one aspect, the nucleic acid disclosed herein comprises from about 20 to about 100 nucleotides.

According to one aspect, a method of altering a human or porcine cell is provided including transfecting the human or porcine cell with a nucleic acid encoding RNA complementary to ASFV viral DNA in the eukaryotic cell, transfecting the human or porcine cell with a nucleic acid encoding an enzyme that interacts with the RNA and cleaves the genomic DNA in a site-specific manner, wherein the human or porcine cell expresses the RNA and the enzyme, the RNA binds to complementary genomic DNA and the enzyme cleaves the genomic DNA in a site-specific manner. According to one aspect, the enzyme is Cas9 or modified Cas9 or a homolog of Cas9. Modified Cas9 proteins or homologs of Cas9 are for instance disclosed in U.S. Pat. No. 9,074,199, which is incorporated herein by reference. According to one aspect, the enzyme is Cas12a or modified Cas12a or a homolog of Cas12a. According to one aspect, the enzyme is Cas12a2 or modified Cas12a2 or a homolog of Cas12a2. According to one aspect, the enzyme is Cas13b or modified Cas13b or a homolog of Cas13b. According to one aspect, the enzyme is CasX or modified CasX or a homolog of CasX. According to one aspect, the RNA includes between about 10 to about 250 nucleotides. According to one aspect, the RNA includes between about 20 to about 100 nucleotides. The step of transfecting a nucleic acid encoding an RNA may be added to any method disclosed herein so that there is sequential or concurrent transfection of not only synthetic guide or tracer sequences such as those disclosed herein but also one or a plurality of vectors comprising a nucleic acid sequence encoding a Cas protein or variant or functional fragment thereof. The disclosure relates, among other things, to the rationale design of sgRNA, tracr/crRNA duplexes, and, generally, guide sequences that activate and/or catalyze the reaction of a CRISPR enzyme with a target nucleic acid sequence. The disclosure relates to the discovery that guide sequences (whether in the form of sgRNA, tracr/crRNA duplexes, or tracr/crRNA single strands) can be heavily modified to enhance on-target enzymatic efficiency as long as certain nucleotides that bind to the CRISPR enzyme, variant or functional fragments thereof are conserved at certain positions and/or, in some cases, conserved in respect to certain substituents on each nucleotide that are capable of binding a Cas protein, variant or functional fragments thereof in the presence of such a the Cas protein, variant or functional fragments thereof. Certain positions of the guide sequence can be more heavily modified based upon their functional association to other components of the CRISPR complex. For instance, in some embodiments, the composition or pharmaceutical composition disclosed herein comprises one or a plurality of nucleic acid sequences on one or plurality of nucleic acid molecules wherein the nucleic acid sequences comprise contiguous domains in the 5′ to 3′ orientation: a DNA-targeting domain, a Cas-binding domain, and a transcription terminator domain. Pharmaceutical compositions also provided herein are pharmaceutical compositions comprising a guide or tracrRNA as disclosed herein, or pharmaceutically acceptable salts thereof; and a pharmaceutically acceptable carrier. Thus, in various embodiments, disclosed are pharmaceutical compositions comprising a therapeutically effective amount of at least one disclosed nucleic acid sequence (e.g., SEQ ID NO: 1 through SEQ ID NO:61, and a pharmaceutically acceptable carrier. In a further embodiment, a pharmaceutical composition can be provided comprising a therapeutically effective amount of at least one disclosed compound. In a still further embodiment, a pharmaceutical composition can be provided comprising a prophylactically effective amount of at least one disclosed compound, wherein the compound comprises a nucleic acid sequence. In yet a further embodiment, the disclosure relates to pharmaceutical compositions comprising a pharmaceutically acceptable carrier and a disclosed compound, wherein the compound is present in an effective amount. In an even further embodiment, the pharmaceutical compositions are useful in inhibiting neurotoxicity in a subject. In a still further embodiment, the pharmaceutical compositions are useful in treating ASFV infection. In a still further embodiment, the pharmaceutical compositions are useful in preventing ASFV infection. In a still further embodiment, the pharmaceutical compositions are useful in preventing an acute ASFV infection following exposure to an active ASFV infectious agent. In some embodiments, the one or plurality of nucleic acid sequences comprise a nucleic acid sequence selected from SEQ ID NO: 1-61, or a variant that comprises at least about 70%, 80%, 87%, 90%, 95%, 96%, 97%, 98%, or 99% sequence identity to a nucleic acid sequence listed above. The present invention also provides vectors comprising any of the nucleic acids described above encoding guide RNAs described herein. In some embodiments, the vector is a plasmid. In some embodiments, the vector is a retrovirus. In some embodiments, the retroviral vector is a lentiviral vector. In some embodiments the vector is an AAV vector. In some embodiments, the first nucleic acid sequence is selected from a sequence comprising at least about 75% sequence identity to any one of SEQ ID NO: 1-61.

In some embodiments, the first nucleic acid sequence comprises a modification described herein, or a salt thereof. Pharmaceutically acceptable salts of the compounds are conventional acid-addition salts or base-addition salts that retain the biological effectiveness and properties of the compounds and are formed from suitable non-toxic organic or inorganic acids or organic or inorganic bases. Exemplary acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Example base-addition salts include those derived from ammonium, potassium, sodium and quaternary ammonium hydroxides, such as for example, tetramethylammonium hydroxide. Chemical modification of a pharmaceutical compound into a salt is a known technique to obtain improved physical and chemical stability, hygroscopicity, flowability and solubility of compounds. See, e.g., H. Ansel et. al., Pharmaceutical Dosage Forms and Drug Delivery Systems (6th Ed. 1995) at pp. 196 and 1456-1457. The pharmaceutical compositions comprise the compounds in a pharmaceutically acceptable carrier. A pharmaceutically acceptable carrier refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. The compounds can be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995. In further embodiments, the pharmaceutical composition is administered to a mammal. In still further embodiments, the mammal is a human. In some embodiments, the mammal is a pig, a swine, or a minipig. In some embodiments, the mammal is a pig. In further embodiments, the pharmaceutical composition is administered following identification of the mammal in need of treatment of a disorder associated signs of neurological dysfunction. In still further embodiments, the mammal has been diagnosed with a need for treatment of a disorder associated with ASFV prior to the administering step. In further embodiments, the pharmaceutical composition is administered following identification of the mammal in need of treatment of an ASFV infection. In still further embodiments, the mammal has been diagnosed with a need for treatment of an ASFV infection prior to the administering step. In various embodiments, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy. The choice of carrier will be determined in part by the particular method used to administer the composition. Accordingly, there is a wide variety of suitable formulations of the pharmaceutical composition of the present invention. The following formulations for oral, aerosol, parenteral, subcutaneous, intravenous, intraarterial, intramuscular, intraperitoneal, intrathecal, rectal, and vaginal administration are merely exemplary and are in no way limiting. Formulations suitable for oral administration can comprise (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granule; (c) powders; (d) suspensions in an appropriate liquid; and (e) suitable emulsions. Liquid formulations may include diluents, such as water, cyclodextrin, dimethyl sulfoxide and alcohols, for example, ethanol, benzyl alcohol, propylene glycol, glycerin, and the polyethylene alcohols including polyethylene glycol, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, the addition to the active ingredient in an inert base, such as gelatin and glycerin, or sucrose and acadia, emulsions, and gels containing, in addition to the active ingredient, such carriers as are known in the art. The compounds of the present disclosure alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer. Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethylene glycol) 400, glycerol ketals, such as 2,2-dimethyl-1, 3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropyl methylcellulose, or carboxymethylcellulose, or emulsifying agents and other pharmaceutical adjuvants. Oils which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isosteric acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example. dimethyl dialkylammonium halides, and alkyl pyridinium halides, (b) anionic detergents such as, for example, alkyl, aryl, and olefin sulfonates, alkyl olefin, ether, and monoglyceride sulfates, and sulfosuccinates, (c) nonionic detergents such as, for example, fatty amine oxides, fatty acid alkanolamines, and polyoxymethylene polypropylene copolymers, (d) amphoteric detergents such as, for example, alkyl n-amino propionates, and 2-alkylimidazoline quaternary ammonium salts, and (e) mixtures thereof. The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present disclosure. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J.B. Lippincott Co., Philadelphia, PA, Banker and Chalmers, Eds., 238-250 (1982) and ASHP Handbook on Injectable Drugs, Toissel, 4^{th} ed., 622-630 (1986). Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, and gels containing, in addition to the active ingredient, such carriers as are known in the art. Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate. One skilled in the art will appreciate that suitable methods of exogenously administering a compound of the present disclosure to an animal are available, and, although more than one route can be used to administer a particular compound, a particular route can provide a more immediate and more effective reaction than another route. As regards these applications, the present method includes the administration to an animal, particularly a mammal, and more particularly a human, of a therapeutically effective amount of the compound effective in the treatment (e.g., prophylactic or therapeutic) of an ASFV infection. The method also includes the administration of a therapeutically effect amount of the compound for the treatment of patient having a predisposition for being afflicted with an ASFV infection. The dose administered to an animal, particularly a human, in the context of the present invention should be sufficient to affect a therapeutic response in the animal over a reasonable timeframe. One skilled in the art will recognize that dosage will depend upon a variety of factors including the condition of the animal, the body weight of the animal, as well as the severity and stage of the disorder. The total amount of the compound of the present disclosure administered in a typical treatment is preferably from about 1 mg/kg to about 100 mg/kg of body weight for mice, and from about 10 mg/kg to about 50 mg/kg of body weight, and from about 20 mg/kg to about 40 mg/kg of body weight for humans per daily dose. This total amount is typically, but not necessarily, administered as a series of smaller doses over a period of about one time per day to about three times per day for about 24 months, and over a period of twice per day for about 12 months. The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations. In certain embodiments, a composition described herein is formulated for administration to a patient in need of such composition. Compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the compositions described herein may be aqueous or oleaginous suspension. These suspensions may be formulated according to techniques known in the art using suitable dispersing or wetting agents and suspending agents.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition. A compound described herein can be administered alone or can be co-administered with an additional therapeutic agent. Thus, the preparations can also be combined, when desired, with other active substances (e.g., to reduce metabolic degradation). Additional therapeutic agents include, but are not limited to, other active agents known to be useful in treating an ASFV infection as further described herein. In some embodiments, the compounds described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.). Suitable compositions include, but are not limited to, oral non-absorbed compositions. Suitable compositions also include, but are not limited to saline, water, cyclodextrin solutions, and buffered solutions of pH 3-9. The compounds described herein, or pharmaceutically acceptable salts thereof, can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH7.0), 0.9% saline, 1.2% saline, acetate, aspartate, benzenesulfonate, benzoate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, succinate, sulfate, tartrate, teoclate, tosylate, and any combination thereof. In some embodiments, an excipient is chosen from propylene glycol, purified water, and glycerin. In some embodiments, the formulation can be lyophilized to a solid and reconstituted with, for example, water prior to use. When administered to a mammal (e.g., to an animal such as a pig for veterinary use or to a human for clinical use) the compounds can be administered in isolated form. When administered to a pig or a human, the compounds can be sterile. Water is a suitable carrier when the compound of Formula I is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents. The compositions described herein can take the form of a solution, suspension, emulsion, tablet, pill, pellet, capsule, capsule containing a liquid, powder, sustained-release formulation, suppository, aerosol, spray, or any other form suitable for use. Examples of suitable pharmaceutical carriers are described in Remington's Pharmaceutical Sciences, A. R. Gennaro (Editor) Mack Publishing Co. In some embodiments, the compounds are formulated in accordance with routine procedures as a pharmaceutical composition adapted for administration to humans. In some embodiments, the compounds are formulated in accordance with routine procedures as a pharmaceutical composition adapted for administration to domesticated animals such as pigs. Typically, compounds are solutions in sterile isotonic aqueous buffer. Where necessary, the compositions can also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound is to be administered by infusion, it can be dispensed, for example, with an infusion bottle containing sterile pharmaceutical grade water or saline. Where the compound is administered by injection, an ampoule of sterile water for injection or saline can be provided so that the ingredients may be mixed prior to administration. The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms. In some embodiments, a composition of the present disclosure is in the form of a liquid wherein the active agent is present in solution, in suspension, as an emulsion, or as a solution/suspension. In some embodiments, the liquid composition is in the form of a gel. In other embodiments, the liquid composition is aqueous. In other embodiments, the composition is in the form of an ointment. In some embodiments, the composition is in the form of a solid article. For example, in some embodiments, the ophthalmic composition is a solid article that can be inserted in a suitable location in the eye, such as between the eye and eyelid or in the conjunctival sac, where it releases the active agent as described, for example, U.S. Pat. Nos. 3,863,633; 3,867,519; 3,868,445; 3,960,150; 3,963,025; 4,186,184; 4,303,637; 5,443,505; and 5,869,079. Release from such an article is usually to the cornea, either via the lacrimal gland that bathes the surface of the cornea, or directly to the cornea itself, with which the solid article is generally in intimate contact. Solid articles suitable for implantation in the eye in such fashion are generally composed primarily of polymers and can be bioerodible or non-bioerodible. Bioerodible polymers that can be used in the preparation of ocular implants carrying one or more of the compounds described herein in accordance with the present disclosure include, but are not limited to, aliphatic polyesters such as polymers and copolymers of poly(glycolide), poly(lactide), poly(epsilon-caprolactone), poly-(hydroxybutyrate) and poly(hydroxyvalerate), polyamino acids, polyorthoesters, polyanhydrides, aliphatic polycarbonates and polyether lactones. Suitable non-bioerodible polymers include silicone elastomers. The compositions described herein can contain preservatives. Suitable preservatives include, but are not limited to, mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and sorbic acid and salts thereof. Compositions of the disclosure include particles comprising the nucleic acid sequences and/or molecule disclosed herein. As used herein, a “particle” refers to any entity having a diameter of less than 100 microns (μm). Typically, particles have a longest dimension (e.g., diameter) of 1000 nm or less. In some embodiments, particles have a diameter of 300 nm or less. In some embodiments, nanoparticles have a diameter of about 200 nm or less. In some embodiments, nanoparticles have a diameter of about 100 nm or less. In general, particles are greater in size than the renal excretion limit but are small enough to avoid accumulation in the liver. In some embodiments, a population of particles may be relatively uniform in terms of size, shape, and/or composition. In general, inventive particles are biodegradable and/or biocompatible. Inventive particles can be solid or hollow and can comprise one or more layers. In some embodiments, particles are spheres, spheroids, flat, plate-shaped, cubes, cuboids, ovals, ellipses, cylinders, cones, or pyramids. In some embodiments, particles can be a matrix of polymers. In some embodiments, the matrix is cross-linked. In some embodiments, formation of the matrix involves a cross-linking step. In some embodiments, the matrix is not substantially cross-linked. In some embodiments, formation of the matrix does not involve a cross-linking step. In some embodiments, particles can be a non-polymeric particle (e.g., a metal particle, quantum dot, ceramic, inorganic material, bone, etc.). Components of the pharmaceutical compositions disclosed herein may comprise particles or may be microparticles, nanoparticles, liposomes, and/or micelles comprising one or more disclosed nucleic acid sequences. As used herein, the term “nanoparticle” refers to any particle having a diameter of less than 1000 nm. Examples of nanoparticles are disclosed in Nature Biotechnology 31, 638-646, which is herein incorporated by reference in its entirety. It is understood that the disclosed compositions can be prepared from the disclosed compounds. It is also understood that the disclosed compositions can be employed in the disclosed methods of use.

### Kits

In some aspects, disclosed are kits comprising a nucleic acid described herein, or a pharmaceutically acceptable salt thereof, and one or more selected from: (a) instructions for treating an ASFV infection; and (b) instructions for administering the nucleic acid in connection with treating ASFV. In some embodiments, a pharmaceutical composition comprising a nucleic acid described herein can be packaged in a container. In some embodiments, the instructions can direct administration of the pharmaceutical composition. In some embodiments, the kit further comprises a drug delivery device for administering the pharmaceutical composition to a subject. In some embodiments, the drug delivery device is a syringe or a catheter. In further embodiments, the kit comprises the agent known for the treatment of an ASFV infection. Examples of agents known for the treatment of an ASFV infection include, but are not limited to, CRISPR enzyme and a guide RNA comprising a DNA-binding region complementary to one or more ASFV genes. In further embodiments, the compound and at least one agent are co-formulated. In further embodiments, the compound and at least one agent are co-packaged. The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient. It is understood that the disclosed kits can be prepared from the disclosed compounds, products, and pharmaceutical compositions. It is also understood that the disclosed kits can be employed in connection with the disclosed methods of use. Methods of making a kit can include placing a pharmaceutical composition comprising a nucleic acid described herein, salt thereof, formulation, or composition described herein in a container for packaging. A method can further comprise an inclusion of instructions for use.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the disclosure concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the disclosure to the form disclosed herein. Also, it is intended to the appended claims be construed to include alternative embodiments. All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### Definitions

Listed below are definitions of various terms used to describe this invention. These definitions apply to the terms as they are used throughout this specification, unless otherwise limited in specific instances, either individually or as part of a larger group.

As used herein, the terms “a” or “an” means that “at least one” or “one or more” unless the context clearly indicates otherwise. The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in various embodiments, to A without B (optionally including elements other than B); in another embodiment, to B without A (optionally including elements other than A); in yet another embodiment, to both A and B (optionally including other elements); etc. The term “or” as used herein shall only be interpreted as indicating exclusive alternatives (i.e., “one or the other but not both”) when preceded by terms of exclusivity, “either,” “one of,” “only one of,” or “exactly one of”.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, “either,” “one of,” “only one of,” or “exactly one of.” “Consisting essentially of,” when used in the claims, shall have its ordinary meaning as used in the field of patent law.

The term “about” as used herein when referring to a measurable value such as an amount, a temporal duration, and the like, is meant to encompass variations of ±20%, ±10%, ±5%, ±1%, ±0.9%, ±0.8%, ±0.7%, ±0.6%, ±0.5%, ±0.4%, 0.3%, ±0.2% or ±0.1% from the specified value, as such variations are appropriate to perform the disclosed methods.

As used herein, the term “kit” refers to a set of components provided in the context of a system for delivering materials or diagnosing a subject with having been contaminated with a disclosed toxin or exposed to a disclosed toxin. Such delivery systems may include, for example, systems that allow for storage, transport, or delivery of various therapeutic reagents (e.g., oligonucleotides, enzymes, extracellular matrix components etc. in appropriate containers) and/or supporting materials (e.g., buffers, media, cells, written instructions for performing the assay etc.) from one location to another. For example, in some embodiments, kits include one or more enclosures (e.g., boxes) containing relevant reaction reagents and/or supporting materials. As used herein, the term “fragmented kit” refers to a kit comprising a therapeutically effective amount of the nucleic acid disclosed herein and wherein the kit comprises two or more separate containers that each contain a sub-portion of total kit components. Containers may be delivered to an intended recipient together or separately. For example, a first container may contain a petri dish or polystyrene plate for use in a cell culture assay, while a second container may contain an excipient or liposome. As another example, the kit may comprise a first container comprising a solid support such as a chip or slide with one or a plurality of ligands with affinities to one or a plurality of biomarkers disclosed herein (such as p72 of ASFV or other capsid protein correlated to viral load) and a second container comprising any one or plurality of reagents necessary for the detection and/or quantification of the amount of biomarkers in a sample.

The term “fragmented kit” is intended to encompass kits containing Analyte Specific Reagents (ASR's) regulated under section 520(e) of the Federal Food, Drug, and Cosmetic Act, but are not limited thereto. Indeed, any delivery system comprising two or more separate containers that each contain a sub-portion of total kit components are included in the term “fragmented kit.” In contrast, a “combined kit” refers to a delivery system containing all components in a single container (e.g., in a single box housing each of the desired components). The term “kit” includes both fragmented and combined kits. In some embodiments, the kit comprises nucleic acids disclosed herein and, optionally, a container comprising a Cas protein or a nucleic acid sequence encoding one or a plurality of Cas proteins.

As used herein, the phrase “integer from about X to about Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

As used herein, “cell culture” means growth, maintenance, transfection, or propagation of cells, tissues, or their products.

As used herein, “culture medium” refers to any solution capable of sustaining the growth of the targeted cells either in vitro or in vivo, or any solution with which targeted cells or exogenous nucleic acids are mixed before being applied to cells in vitro or to a patient in vivo. In some embodiments, culture medium means solution capable of sustaining the growth of the targeted cells either in vitro.

As used herein, the term “animal” includes, but is not limited to, humans and non-human vertebrates such as wild animals, rodents, such as rats, ferrets, and domesticated animals, and farm animals, such as dogs, cats, horses, pigs, cows, sheep, and goats. In some embodiments, the animal is a mammal. In some embodiments, the animal is a human. In some embodiments, the animal is a pig, a swine, or a minipig.

As used herein, the term “mammal” means any animal in the class Mammalia such as rodent (i.e., a mouse, a rat, or a guinea pig), a monkey, a cat, a dog, a cow, a horse, a pig, or a human. In some embodiments, the mammal is a human. In some embodiments, the mammal refers to any non-human mammal. The present disclosure relates to any of the methods or compositions of matter disclosed herein wherein the sample is taken from a mammal or non-human mammal. The present disclosure relates to any of the methods or compositions of matter disclosed herein wherein the sample is taken from a human, pig or minipig.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Nat. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001.As used herein, “specific for” or “specifically binds to” means that the binding affinity of a substrate to a specified target nucleic acid sequence, such as a nucleic acid that encodes an ASFV enzyme or structural protein, is statistically higher than the binding affinity of the same substrate to a generally comparable, but non-target amino acid sequence. The substrate's Kd to each nucleotide sequence can be compared to assess the binding specificity of the substrate to a particular target nucleotide sequence.

Human or non-human variants of the enzymes above are contemplated by the methods, systems, and devices disclosed herein. Variants of these enzymes include sequences that comprise at least 70% of the sequence to the porcine or human sequences disclosed herein. As used herein, the term “variants” is intended to mean substantially similar sequences. For nucleic acid molecules, a variant comprises a nucleic acid molecule having deletions (e.g., truncations) at the 5′ and/or 3′ end; deletion and/or addition of one or more nucleotides at one or more internal sites in the native polynucleotide; and/or substitution of one or more nucleotides at one or more sites in the native polynucleotide.

As used herein, a “native” nucleic acid molecule or polypeptide comprises a naturally occurring nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule or amino acid sequence of the disclosure will have at least about 70%, 75%, 80%, 85%, 87%, 90%, 91%, 92%, 93%, 94%, 95%, 96%, 97%, 98%, 99% or more sequence identity to that particular polynucleotide or guide RNA or target DNA as determined by sequence alignment programs and parameters as described elsewhere herein. Variants of a particular nucleic acid molecule of the disclosure (e.g., the reference amino acid sequence) can also be evaluated by comparison of the percent sequence identity between the polypeptide encoded by a variant nucleic acid molecule and the polypeptide encoded by the reference nucleic acid molecule comprising an expressible nucleic acid sequence. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 87%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins, variant guide RNAs or nucleic acid sequences encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 87%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as about 1 to about 15 amino acid residues, as few as about 1 to about 15, such as from about 6 to about 10, as few as about 20, 15, 10, 9, 8, 7, 6, 5, as few as about 4, 3, 2, or even about 1 amino acid residue. The nucleic acid sequences or molecules as well as the proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly.

The abbreviations used herein have their conventional meaning within the chemical and biological arts.

The chemical structures and formulae set forth herein are constructed according to the standard rules of chemical valency known in the chemical arts.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X, and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” mean that the subsequently described event or circumstance can or cannot occur, and that the description includes instances where said event or circumstance occurs and instances where it does not.

As used herein, the term “diagnosed” means having been subjected to a physical examination by a person of skill, for example, a physician or veterinarian, and found to have a condition that can be diagnosed or treated by the compounds, compositions, or methods disclosed herein. In some embodiments of the disclosed methods, the subject has been diagnosed with a need for treatment of a disorder caused by ASFV such as, for example, a fever, prior to the administering step. As used herein, the phrase “identified to be in need of treatment for a disorder,” or the like, refers to selection of a subject based upon need for treatment of the disorder. It is contemplated that the identification can, in some embodiments, be performed by a person different from the person making the diagnosis. It is also contemplated, in further embodiments, that the administration of a diagnosis can be performed by one who previously or subsequently performed the administration of a pharmaceutical composition disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intra-aural administration, intracerebral administration, rectal administration, and parenteral administration, including injectable such as intravenous administration, intra-arterial administration, intramuscular administration, and subcutaneous administration. Administration can be continuous or intermittent. In various embodiments, a preparation can be administered therapeutically; that is, administered to treat an existing disease or condition. In further various embodiments, a preparation can be administered prophylactically; that is, administered for prevention of a disease or condition.

As used herein, the terms “contacting” or “exposing” mean bringing together of two elements in an in vitro system or an in vivo system. For example, “contacting” a compound disclosed herein with an individual or subject or cell includes the administration of the compound to an individual or subject, such as a pig, as well as, for example, introducing a compound into a sample containing a cellular or purified preparation containing the compounds or pharmaceutical compositions disclosed herein.

The nucleic acid sequences according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context refer to derivatives falling within at least two of the groups: pharmaceutically acceptable salts, prodrugs, deuterated forms, radio-actively labeled forms, isomers, and solvates. Examples of radio-actively labeled forms of nucleic acid sequences include compounds labeled with tritium, phosphorous-32, iodine-129, carbon-11, Feverorine-18, and the like. Methods of tracking the presence or location of the nucleic acids or proteins of certain embodiments comprise detecting the amount of radioactive energy emitted from the labeled nucleic acids or proteins.

“Therapeutically effective amount” refers to an amount of a compound, material, or composition, as described herein effective to achieve a particular biological result such as, but not limited to, biological results disclosed, described, or exemplified herein. Such results may include, but are not limited to, the effective reduction of symptoms associated with any of the disease states mentioned herein, as determined by any means suitable in the art. The therapeutically effective amount of the composition may be dependent on any number of variables, including without limitation, the species, breed, size, height, weight, age, overall health of the subject, the type of formulation, the mode or manner or administration, the type and/or severity of the particular condition being treated, or the need to modulate the activity of the molecular pathway induced by association of the guide RNA or tracer RNA to the viral DNA target. The therapeutically appropriate effective amount can be routinely determined by those of skill in the art using routine optimization techniques and the skilled and informed judgment of the practitioner and other factors evident to those skilled in the art. A therapeutically effective dose of the saxiphilins described herein may provide partial or complete biological activity as compared to the biological activity induced by the wild-type or naturally occurring polypeptides upon which the saxiphilins are derived. A therapeutically effective dose of the proteins or amino acids described herein may provide a sustained biochemical or biological affect and/or an increased resistance to infection of ASFV when administered to a subject as compared with the normal affect observed in the absence of the administration. In some embodiments, the term “effective amount” or “therapeutically effective amount” refers to an amount that is sufficient to achieve the desired result (e.g., that will elicit a biological or medical response of a subject e.g., a dosage from about 0.01 to about 100 mg/kg body weight/day) or to have an effect on an undesired condition. For example, a “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various embodiments, a preparation can be administered in a “prophylactically effective amount”; that is, an amount effective for prevention of a disease or condition. The term “fragment” refers to any analog of a naturally occurring polypeptide disclosed herein that comprises at least 4 amino acids identical to the naturally occurring polypeptide upon which the analog is based.

The term “functional fragment” refers to any fragment of Cas protein disclosed herein that comprises at least about 75% sequence identity to any of those amino acid sequence chosen from Table Y, and shares the function of the naturally occurring polypeptide upon which the saxiphilin is based. In some embodiments, the functional nature of the fragment is to bind or associate a disclosed DNA target sequence, and, in some embodiments, cut the DNA target sequence if within a cell, in vitro or in a subject such as a pig.

The compounds described herein may be present in the form of pharmaceutically acceptable salts. For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include (e.g., salts of inorganic acids such as hydrochloric acid, hydrobromic, phosphoric, nitric, and sulfuric acids and of organic acids such as acetic acid, benzenesulfonic, benzoic, methanesulfonic, and p-toluenesulfonic acids). Examples of pharmaceutically acceptable base addition salts include e.g., sodium, potassium, calcium, ammonium, organic amino, or magnesium salt. The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The pharmaceutical composition can comprise any pharmaceutically acceptable carrier or ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. As used herein, the phrase “pharmaceutically acceptable” means those compounds, materials, compositions, and/or dosage forms which are, within the scope of sound medical judgment, suitable for use in contact with tissues of humans or other animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

“Disease” or “condition” refer to a state of being or health status of a patient or subject capable of being treated with a compound, pharmaceutical composition, or method provided herein. In some embodiments, the disease is a disease related to (e.g., characterized by) modulation of ASFV viral load. In some embodiments, the disease is an ASFV infection. “Disease”, “disorder”, and “condition” are used interchangeably herein. In some embodiments, the condition is an ASFV infection. As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed, (e.g., a therapeutic treatment). In other embodiments, treatment may be administered in the absence of symptoms. For example, treatment may be administered to a susceptible subject prior to the onset of one or more symptoms (e.g., in light of a history of symptoms and/or in light of exposure to a particular organism such as ASFV, or other susceptibility factors), as a prophylactic treatment. Treatment may also be continued after symptoms have resolved, for example to delay their recurrence.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. The term “preventing” refers to preventing a disease, disorder, or condition from occurring in a human or an animal that may be predisposed to the disease, disorder and/or condition, but has not yet been diagnosed as having it; and/or inhibiting the disease, disorder, or condition, i.e., arresting its development. In some embodiments the disclosure relates to a method of preventing ASFV viral replication or viral assembly or viral particle release an infected cell by administering to a subject a therapeutically effective amount of a Cas protein and a disclosed nucleic acid sequence (such as a guide or tracrRNA) comprising a sequence that associates or hybridizes a DNA target sequence of an ASFV genomic DNA or endogenous sequence. Nucleic acids that hybridize with a DNA target sequence are exemplified in Table 2. In some embodiments, the viral endogenous or genomic sequence is in the cytosol of the infected cell. In some embodiments, the endogenous or genomic sequence is in the nucleus of an infected cell.

As used herein, the term “salt” refers to acid or base salts of the compounds used in the methods of the present disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. The term “salt” refers to acidic salts formed with inorganic and/or organic acids, as well as basic salts formed with inorganic and/or organic bases. Examples of these acids and bases are well known to those of ordinary skill in the art. Such acid addition salts will normally be pharmaceutically acceptable although salts of non-pharmaceutically acceptable acids may be of utility in the preparation and purification of the compound in question. Salts include those formed from hydrochloric, hydrobromic, sulfuric, phosphoric, citric, tartaric, lactic, pyruvic, acetic, succinic, fumaric, maleic, methanesulfonic and benzenesulfonic acids. In some embodiments, salts of the compositions comprising a saxiphilin or functional fragment thereof may be formed by reacting the free base, or a salt, enantiomer or racemate thereof, with one or more equivalents of the appropriate acid. In some embodiments, pharmaceutical acceptable salts of the present invention refer to analogs having at least one basic group or at least one basic radical. In some embodiments, pharmaceutical acceptable salts of the present invention comprise a free amino group, a free guanidino group, a pyrazinyl radical, or a pyridyl radical that forms acid addition salts. In some embodiments, the pharmaceutical acceptable salts of the present invention refer to analogs that are acid addition salts of the subject compounds with (for example) inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxybenzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-2-sulfonic acid. When several basic groups are present mono- or poly-acid addition salts may be formed. The reaction may be carried out in a solvent or medium in which the salt is insoluble or in a solvent in which the salt is soluble, for example, water, dioxane, ethanol, tetrahydrofuran or diethyl ether, or a mixture of solvents, which may be removed in vacuo or by freeze drying. The reaction may also be a metathetical process or it may be carried out on an ion exchange resin. In some embodiments, the salts may be those that are physiologically tolerated by a patient. Salts according to the present invention may be found in their anhydrous or hydrated crystalline form (i.e., complexed or crystallized with one or more molecules of water).

The terms “subject” and “patient” and “subject in need thereof” may be used interchangeably, and means a mammal in need of treatment, e.g., companion animals (e.g., dogs, cats, and the like), farm animals (e.g., cows, pigs, minipigs, horses, sheep, goats and the like), laboratory animals (e.g., rats, mice, guinea pigs and the like), and wild animal populations (e.g., wild boar (Sos scrofa, or any one of a recognized subspecies of Sos scrofa)). Non-limiting examples include humans, other mammals, bovines, cats, rats, mice, dogs, monkeys, goats, sheep, cows, horses, pigs, and other non-mammalian animals. In some embodiments, the subject is a pig, a swine, or a minipig in need of treatment for ASFV or neurotoxicity.

The term “associated” or “associated with” in the context of a substance or substance activity or function associated with a disease (e.g., a protein associated disease, a symptom associated with ASFV infection) means that the disease (e.g., the ASFV) is caused by (in whole or in part), or a symptom of the disease is caused by (in whole or in part) the substance or substance activity or function. For example, a symptom of a gut motility disease or condition may be a symptom that results (entirely or partially) from modulation of viral load (e.g., induction of colonic motility). As used herein, what is described as being associated with a disease, if a causative agent, could be a target for treatment of the disease. For example, ASFV infection may be treated with an agent (e.g., compound as described herein) effective for modulating inflammation or fever within a subject, such as a pig.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be an amino acid sequence disclosed herein and a nucleic acid sequence disclosed herein in complex with viral genomic DNA. In some embodiments contacting includes allows the CRISPR complex described herein to interact with a viral DNA and neutralize or inhibit its biological effect or effects. In some embodiments, the biological effect is the reduction of viral load in a subject and/or cell.

As defined herein, the term “inhibition,” “inhibit,” “inhibiting,” and the like in reference to a protein-inhibitor (e.g., antagonist) interaction means negatively affecting (e.g., decreasing) the activity or function of the protein relative to the activity or function of a viral protein or viral genome in the absence of the inhibitor. In some embodiments inhibition refers to reduction of a disease or symptoms of disease caused by the presence or infection of the virus. In some embodiments, inhibition refers to a reduction in the activity of a signal transduction pathway or signaling pathway caused by the presence of a virus, such as ASFV. Thus, inhibition includes, at least in part, partially or totally blocking infection, decreasing, preventing, or delaying replication or viral assembly, or inactivating, desensitizing, or down-regulating signal transduction or enzymatic activity of the amount of a viral protein in a cell as compared to the same metric in a cell not treated with the same inhibitor.

As defined herein, the term “activation,” “activate,” “activating,” and the like in reference to a protein-activator (e.g., agonist) interaction means positively affecting (e.g., increasing) the activity or function of the protein relative to the activity or function of the protein in the absence of the activator. In some embodiments, activation refers to an increase in the activity of a signal transduction pathway or signaling pathway. Thus, activation may include, at least in part, partially or totally increasing stimulation, increasing or enabling activation, or activating, sensitizing, or up-regulating signal transduction or enzymatic activity or the amount of a protein decreased in a disease. Activation may include, at least in part, partially or totally increasing stimulation, increasing or enabling activation, or activating, sensitizing, or up-regulating signal transduction or enzymatic activity or the amount of a protein that may modulate the level of another protein or increase/decrease cell survival.

The term “modulator” refers to a composition that increases or decreases the level of a target molecule or the function of a target molecule. In some embodiments, the modulator is a modulator of neurotoxicity. In some embodiments, the modulator is a modulator of viral toxicity or viral load in an infected cell and is a compound that reduces the severity of one or more symptoms of a disease associated with viral load, such as ASFV. In some embodiments, a modulator is a compound that reduces the severity of one or more symptoms of ASFV infection caused by exposure to or contamination by ASFV.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Oral administration may include adding a composition described herein to a food source of the subject to be injected during feeding. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., viral therapies including, for example, cholinesterase inhibitors, modafinil, or non-steroidal anti-inflammatory drugs. The composition of the disclosure can be administered alone or can be co-administered to the subject. Coadministration is meant to include simultaneous or sequential administration of the compound individually or in combination (more than one compound or agent). Thus, the preparations can also be combined, when desired, with other active substances (e.g., to reduce metabolic degradation). The compositions of the present disclosure can be delivered transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In some embodiments, the formulations of the compositions of the present disclosure can be delivered by the use of liposomes which fuse with the cellular membrane or are endocytosed, (e.g., by employing receptor ligands attached to the liposome, that bind to surface membrane protein receptors of the cell resulting in endocytosis). By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present disclosure can also be delivered as nanoparticles. Pharmaceutical compositions provided by the present disclosure include compositions wherein the active ingredient (e.g., compounds described herein, including embodiments or examples of saxiphilins and functional fragments thereof) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms. Determination of a therapeutically effective amount of a compound of the disclosure is well within the capabilities of those skilled in the art, especially in light of the detailed disclosure herein. The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g., symptoms of ASFV infection), kind of concurrent treatment, complications from the disease being treated or other health-related problems. Other therapeutic regimens or agents can be used in conjunction with the methods and compounds of Applicants' disclosure. Adjustment and manipulation of established dosages (e.g., frequency and duration) are well within the ability of those skilled in the art. For any compound described herein, the therapeutically effective amount can be initially determined from in vitro cell culture assays. Target concentrations will be those concentrations of active compound(s) that are capable of achieving the methods described herein, as measured using the methods described herein or known in the art. As is well known in the art, therapeutically effective amounts for use in animal subjects can also be determined from designed test protocols in animal models. For example, a dose for pigs can be formulated to achieve a concentration that has been found to be effective in designed test protocols in animal models. The dosage in pigs can be adjusted by monitoring compounds effectiveness and adjusting the dosage upwards or downwards, as described above. Adjusting the dose to achieve maximal efficacy in pigs based on the methods described above and other methods is well within the capabilities of the ordinarily skilled artisan. Dosages may vary depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state. Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent. The compounds described herein can be used in combination with one another, with other active agents known to be useful in treating symptoms of ASFV infection as further described herein, or with adjunctive agents that may not be effective alone but may contribute to the efficacy of the active agent. In some embodiments, co-administration includes administering one active agent within about 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, e.g., preparing a single pharmaceutical composition including both active agents. In other embodiments, the active agents can be formulated separately. In some embodiments, the active and/or adjunctive agents may be linked or conjugated to one another. In some embodiments, the compounds described herein may be combined with treatments for general inflammation or infection such as antibiotics for bacterial infection.

As used herein, the term “prodrug” means a derivative of a known direct acting drug, which derivative has enhanced delivery characteristics and therapeutic value as compared to the drug and is transformed into the active drug by an enzymatic or chemical process. The compounds described herein also include derivatives referred to as prodrugs, which can be prepared by modifying functional groups present in the compounds in such a way that the modifications are cleaved, either in routine manipulation or in vivo, to the parent compounds. Examples of prodrugs include compounds of the disclosure as described herein that contain one or more molecular moieties appended to a hydroxyl, amino, sulfhydryl, or carboxyl group of the compound, and that when administered to a patient, cleaves in vivo to form the free hydroxyl, amino, sulfhydryl, or carboxyl group, respectively. Examples of prodrugs include, but are not limited to, acetate, formate and benzoate derivatives of alcohol and amine functional groups in the compounds of the disclosure. Preparation and use of prodrugs is discussed in T. Higuchi et al., “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference in their entireties. As used herein, the term “purified” means that when isolated, the isolate contains at least 90%, at least 95%, at least 98%, or at least 99% of a compound described herein by weight of the isolate.

As used herein, the phrase “solubilizing agent” means agents that result in formation of a micellar solution or a true solution of the drug.

As used herein, the term “solution/suspension” means a liquid composition wherein a first portion of the active agent is present in solution and a second portion of the active agent is present in particulate form, in suspension in a liquid matrix.

As used herein, the phrase “substantially isolated” means a compound that is at least partially or substantially separated from the environment in which it is formed or detected. It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable subcombination. It should be noted that any embodiment of the disclosure can optionally exclude one or more embodiment for purposes of claiming the subject matter. In some embodiments, the nucleic acid sequences that encode Cas proteins are free of a nuclear localization signal or signals. In some embodiments, the compounds, or salts thereof, are substantially isolated. Partial separation can include, for example, a composition enriched in the compound of the disclosure. Substantial separation can include compositions containing at least about 50%, at least about 60%, at least about 70%, at least about 80%, at least about 90%, at least about 95%, at least about 97%, or at least about 99% by weight of the compound of the disclosure, or salt thereof. Methods for isolating compounds and their salts are routine in the art. In some embodiments, methods of the disclosure comprise a step of isolating a nanoparticle or liposome comprising a Cas protein and/or a guide RNA or tracrRNA.

### EXAMPLES

The following illustrative examples are representative of embodiments of the stimulation, systems, and methods described herein and are not meant to be limiting in any way.

### Example 1: Transfection of Cells for In Vitro Disruption of ASFV Infection

### Porcine Macrophage Protocol (for 1000 ml of Blood): Day 1

Materials for use: 50 ml conical tubes (and holder); Ficoll, 250 ml centrifuge bottle; 0.45 and 0.22 filters (3 each); Frozen L929 media stock; Fetal Bovine Serum (Characterized & Gamma Irradiated); Anti-Anti; Gentamicin.

Wash Media: 500 ml RPMI; 5 ml Anti/Anti; 0.5 ml Gentamicin

MØ Media: 250 ml RPMI; 100 ml FBS; 150 ml L929 Media; 5 ml Anti/Anti; 0.5 ml Gentamicin

Complete Media: 350 ml MØ Media; 150 ml Pig Plasma

Steps for Preparation:
1. Gather all media material in hood to bring to room temp (thaw L929 media in water bath as well as FBS if more is needed).
2. Retrieve previous weeks wash and macrophage media from fridge and set aside (will want to use this up first)
3. Once the blood arrives, record the amount received and the pig number on the bottle.
4. Wipe down the bottle with 70% ethanol and incubate in a water bath for at least an hour (up to two depending on how well the separation is).
5. While blood is incubating, retrieve two 0.45 filters and two 0.22 filters.
   a. In one 0.45 filter combine materials for wash media
   b. In the other 0.45 filter combine the macrophage media materials
   c. Use the air pump to filter both and then filter each through the 0.22 filters.
   d. Label the respective bottles ‘Wash Media’ and ‘MØ Media’ and add date.

The following protocol is followed to collect white blood cells:
1. Separated blood is brought to the hood and 10 conical tubes are uncapped. In some instances, 35 ml of white blood cells in each are aliquoted).
2. With a 25 ml pipet, 35 mL is collected in each tube (keeping the pipet at the surface of the white blood cells and pipet very slowly when approaching the red layer, any mixing was avoided).
3. 30 ml of red cell layer is pipetted in a separate 50 ml tube to wash and dilute later for titrations.

A Ficoll gradient is utilized for the next step:
1. Using a 10 ml pipet, 13 ml of ficoll is aspirated and 10 mL of it dispensed with the tip at the bottom of each of the white blood cell tubes. This extra ficoll is to ensure a smooth gradient and avoid having to turn the pipet motor on to dispense a full 10 ml and cause mixing.
2. Step 1 is repeated with all tubes, changing the pipet tip if the bottle of ficoll will not be finished.
3. Spin gradients are generated for 25-30 min at 1350 rpm with acceleration and deceleration at 0.

The following protocol is conducted to harvest the plasma and white blood cell layer:
1. Collect the plasma from each tube (down to the 20 ml mark on the tube) into a 250 ml centrifuge bottle.
   a. Balance and spin for 20 min at 7000 rpm and 21° C.
2. The white cell layer lies just above the ficoll (around the 10 ml mark) and there is a red cell pellet at the bottom of the tube beneath the ficoll.
3. Using a 10 ml pipet, swirl around the edges of the tube at the 10 ml mark, where the white blood cell layer is, collecting as much as you can but stopping before you hit the 5 ml mark & the red cell pellet.
4. Dispense the white cells into new conical tubes (around 4 should be enough), trying to keep them under 35 ml each.
5. Retrieve the plasma, MØ media, a 0.45 and a 0.22 filter.
6. Run the plasma through the 0.45 filter, pour 350 ml of macrophage media into the 0.22 filter then add 150 ml of 0.45 filtered plasma to make 500 ml of complete media. Label the bottle ‘Complete Media’ ‘pig #’ ‘date’ and set aside for later.

The following protocol is conducted for washing white and red blood cells:
1. Fill the harvested white cell tubes with wash media up to the 45 ml mark. (Wash #1).
2. Fill the harvested red cell tube with 1×DPBS up to the 45 ml mark. (Wash #1).
3. Invert all tubes to mix thoroughly.
4. Centrifuge for 10 minutes at 1350 rpm. (Additionally, can spin samples for 30 minutes at 1000 rpm during one of the washes.)

For white blood cell pellets: pipet up supernatant until the 5 ml mark; resuspend pellet in remaining liquid; condense tubes by half (transfer the contents of one tube to another); rinse empty tube with wash media and add to condensed tube; fill remaining tubes with wash media up to the 45 ml mark (#2); and invert a few times to mix.

For red blood cell pellets: pipet up supernatant until the 20 ml mark; add 1×DPBS up to the 45 ml mark (#2); and invert a few times to mix.

By following the preceding protocols, 4 tubes of white cells (A, B, C, and D) are resuspended. The following protocol is then conducted: Uncap all tubes, and with a 25 ml pipet, aspirate 30 ml of wash media. Deposit 15 ml into tube A and 15 ml into tube B. Pipette up and down in both tubes to further mix the cells. Transfer the contents of tube A to tube C and the contents of tube B to tube C. With a 10 ml pipet, aspirate 10 ml of wash media and wash it down the sides of tube A. Then move the 10 ml of wash media from tube A to tube B to rinse as well. Take the contents of tube B, and put half in tube C and the other half in tube D. Next, centrifuge for 10 minutes at 1350 rpm. Then, repeat the previous steps for white and red cell pellets to make a total of 3 washes (#3).

For handling the white blood cell pellet, if halving the tubes does not condense the white cell tubes to one final tube, the cells can be condensed further, or an extra wash may be completed. If the pellet is too big, a final sample can be more than one tube. The following protocol is then conducted:
1. Resuspend white cell pellet up to the 40 ml mark with complete media.
2. Seed 10 sterile primeria T75 flasks.
3. Label each flask with ‘MØ’ and ‘Pig #’.
4. Fill each flask with 11 ml of complete media and 4 ml of resuspended cells (for a total volume of 15 ml).
5. Cells placed in an incubator overnight.

For handling the red blood cell pellet, the following protocol is conducted: Make the requested percent red blood cells (this is typically 25% in a conical tube (9 ml concentrated red blood cells+27 ml 1×DPBS)); Label ‘25% Red Blood Cells in 1×DPBS’ ‘Pig #’ ‘Date’ ‘Non-Select’.

### Porcine Macrophage Protocol (for 1000 ml of Blood): Day 2

Materials for use: Unopened bottle of 1×DPBS; EDTA; Fetal Bovine Serum (Characterized & Gamma Irradiated); Wash Media, MØ Media, Complete Media, Plasma; 2000 ml beaker for waste; 4 50 ml conical tubes (and holder); Neubauer chamber slide and coverslip for counting cells.

Incubating cells are checked to ensure they remain adhered and are in good condition.

The following is prepared: Set all media, EDTA, 1×DPBS, and 2000 ml waste beaker inside the hood. Add 10 ml of EDTA to the 1×DPBS and shake to mix. Label the bottle+EDTA.

To rinse and detach cells, the following protocol is conducted:
1. Retrieve flasks and in batches of around 6, dump supernatant into waste beaker.
2. Using a 50 ml pipet, put 10 ml of DPBS+EDTA into each flask, swirl and dump to rinse.
3. Add another 10 ml of DPBS+EDTA to each flask, close lid, and incubate for 15-20 min.
4. Take out a flask and firmly hit your palm against the side a few times.
5. If the cells look ready to harvest, do this to all flasks. If not, incubate for longer.
6. Take around 5 flasks at a time, uncap them all, and with a 5 ml pipet wash the liquid in each flask down the sides and break up the cells against the bottom of the flask.
7. Deposit the contents of each flask into the 50 ml conical tubes, reaching no more than 35 ml in each tube.
8. Pass 10 ml of DPBS+EDTA through the same flasks to collect any remaining cells.

The following protocol is conducted for white blood cell washes:
1. Balance the 50 ml conical tubes with wash media. (Wash #1)
2. Invert to mix and spin for 10 minutes at 1200 rpm.
3. Dump supernatant into the waste beaker and resuspend pellet with remaining liquid.
4. Add 15 ml wash media to each tube and condense by half.
5. Pass 10 ml of wash media through empty tubes to collect remaining cells and dispense evenly.
6. Fill all remaining tubes with wash media up to the 45 ml mark. (Wash #2)
7. Repeat steps #2 and #3.
8. Add 15 ml wash media to each tube and condense to one final tube.
9. Pass 10 ml of wash media through empty tubes to collect remaining cells.
10. Make sure the final volume is 40 ml.

For counting cells, the following is prepared: Retrieve a clean Eppendorf tube and rack; Place 100 μL of cells and 900 μL of wash media into the Eppendorf tube; Vortex well and deposit 15 μL of the mixture to both notched sides of the neubauer chamber beneath the coverslip; #Cells×10(dilution factor)×10,000(chamber factor)=Cells/ml volume. X 40 ml=#cells total.

The next steps followed are:
11. Repeat steps #2 and #3. (Wash #3)
12. Add complete media to the 40 ml mark.
13. Plate cells as requested.

For cell transfection, the following protocol is conducted. For transfection, Lipofectamine 2000 (Invitrogen, Carlsbad, CA, USA) is diluted in the Opti-MEM medium and plasmid DNA (CD2V-HA and Empty-HA) are separately diluted in the Opti-MEM medium (Gibco, MA, USA) and incubated for 5 min. About 1 μg of CD2v-HA or Empty-HA plasmid/well (for 6 well plate) is used. Diluted DNA and lipofectamine 2000 are mixed (1:1 ratio) and incubated for 20 min. Finally, the DNA-lipid complex is added to cells for a 5 h incubation then, the Opti-MEM medium is replaced with 10% complete growth media.

The following plasmids are used for cell transfection in this study. Plasmids used in this study contain a cas gene under a CMV/chicken-beta-actin promotor and a crRNA(s) under a U6 promotor and are labeled as follows: 1) P18 VP2 (Cas9_ΔNLS_GFP)pCas9 ASFV scrambled target, 3 sgRNA that do not target anything (thereby serving as a negative control); 2) P41 VP2 (Cas12a2_GFP) Cas12A2_DP_RH_TI, Cas12a2 with sgRNAs (duplicate) targeting three ASFV genes: DNA polymerase, RNA helicase, and topoisomerase II; 3) P2400 VP2 (Cas12a2_ΔNLS_GFP) p41_Cas12a2_DeltaNSL, NLS deletion Cas12a2 with sgRNAs (duplicate) targeting three ASFV genes: DNA polymerase, helicase, and Topoisomerase II. Target sites were selected within ASFV topoisomerase, polymerase, and helicase genes adjacent to valid PAM/PFS sequences. Thus, additional description of the plasmids used in this Example is as follows: 1) P18 VP2 serves as a negative control with scrambled Guide RNA sequences directing the targeting of Cas9 without an NLS; P18 VP2 does contain Cas but is not targeted to ASFV DNA or ASFV RNA indicating its utility as a control. 2) P41 VP2 targets DNA encoding ASFV topoisomerase, polymerase, and helicase genes, and ssRNA complementary to one of strands of DNA encoding ASFV topoisomerase, polymerase, and helicase genes with duplicate Guide RNA sequences directing the targeting of Cas12a2 comprising an NLS to those sequences; and 3) P2400 VP2 targets DNA encoding ASFV topoisomerase, polymerase, and helicase genes, and ssRNA complementary to one of strands of DNA encoding ASFV topoisomerase, polymerase, and helicase genes with duplicate Guide RNA sequences directing the targeting of Cas12a2 that does not comprise a linked NLS to those sequences. Five groups of porcine macrophages are tested (Group 1: MOCK; Group 2: untransfected control; Group 3: P18 VP2; Group 4: P41 VP2; and Group 5: P2400 VP2).

For selection of Guide RNA sequences, potential off-target sites were found by blasting target sequences against Homo sapiens and Sus scrofa. Off-target matches to target sequences of greater than 24/29 nucleotides were omitted. Secondary structure for target sites was analyzed using RNA fold web server by Vienna RNA Web Services.

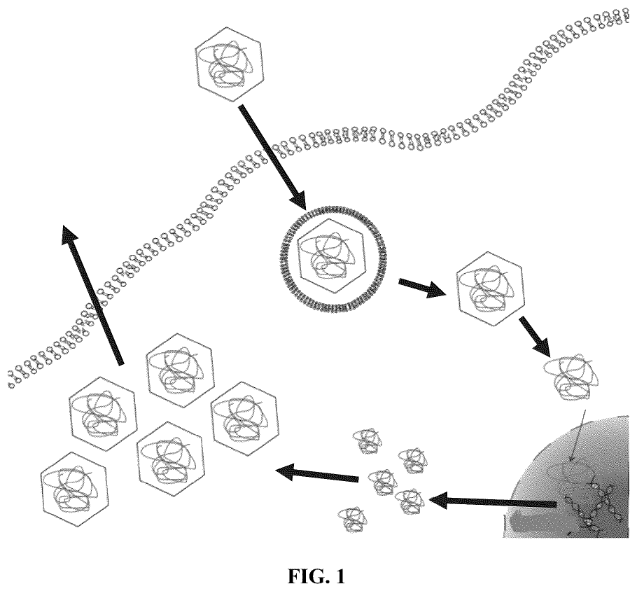

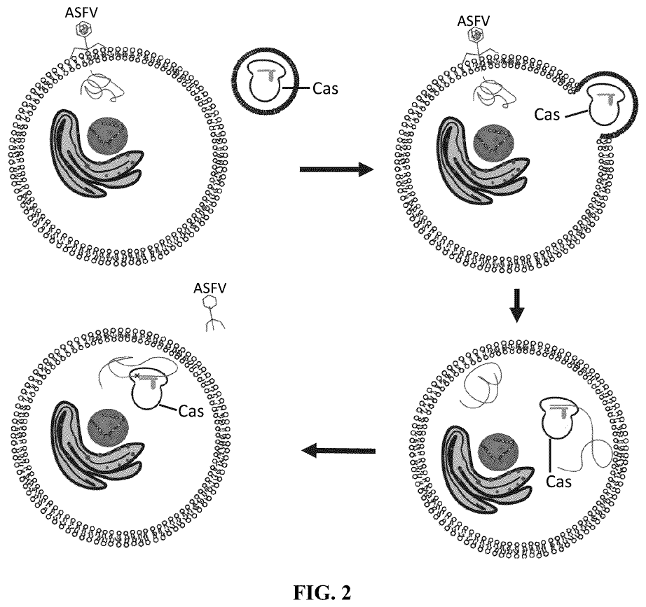

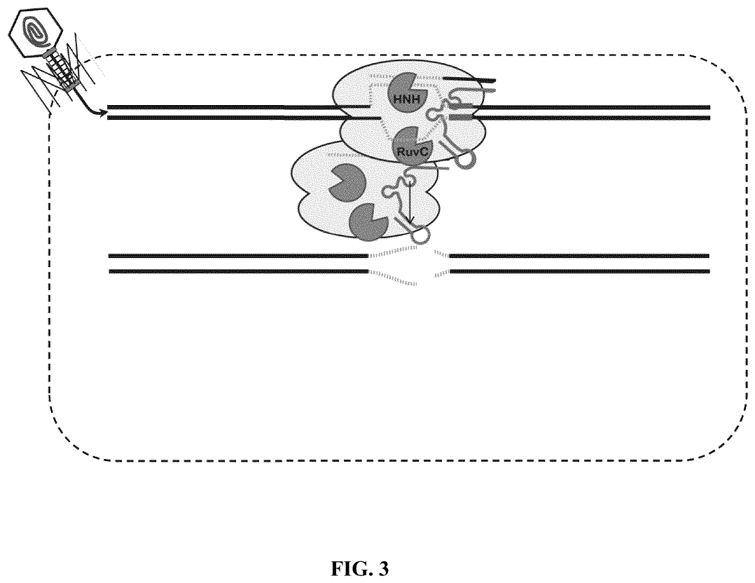

Experiments are conducting testing for prophylactic effects and for treatment effects following ASFV infection by contacting the Test Group cells with active ASFV cultures at different time points in relation to transfection. In Test 1, porcine macrophages from each of Groups 2-5 are contacted with ASFV 48 hours prior to transfection with Group 3, 4, or 5 vectors in respective Groups 3, 4, or 5. In Test 2, porcine macrophages from each of Groups 2-5 are contacted with ASFV at the same time as transfection with Group 3, 4, or 5 vectors in respective Groups 3, 4, or 5. In Test 3, porcine macrophages from each of Groups 2-5 are contacted with ASFV 24 hours following transfection with Group 3, 4, or 5 vectors in respective Groups 3, 4, or 5. 48 hours following transfection, macrophages are assayed in Tests 1-3. Therapeutic transfection efficiency is determined by assaying transfected plasmid expressing Green Fluorescent Protein (GFP) and quantified using flow cytometry. Using immunohistochemistry to detect ASFV p72 protein in treated cells hours after transfection, the amount of virus in the cells as compared to the number of cells that were successfully transfected is calculated. p72-labeled cells are sorted and quantified using flow cytometry and immunofluorescent detection of p72 protein. Groups there were contacted by ASFV were contacted with ASFV E70MS at MOI 1 without inoculum and incubated for an additional 48 hours at 37° C. Test 1 and Test 2 cells were tested 48 hours after transfection. Test 3 cells were tested 96 hours after transfection. FIG. 1 depicts a schematic of the ASFV life cycle in a porcine cell infected by ASFV. The life cycle depicts infection, viral DNA being copied and translated, viral assembly and then release to inform conclusions based in the timing of treatment and infection in this Example. FIG. 2 depicts a schematic of the steps behind the mechanism of action for therapeutic nucleic acids described herein. The panel on the top left of the figure shows a liposome particle comprising Cas protein and one or plurality of disclosed nucleic acid sequences and a cell infected with ASFV. The top right panel depicts the liposome fusing to the infected cell and releasing its contents into the infected cell. The bottom left panel depicts the Cas protein machinery and the disclosed embodiments of nucleic acid sequences binding to the viral genome (indicated by looped line). The bottom right panel shows a cleavage of the viral genome after binding to the Cas protein and enzymatic action of the Cas protein and nucleic acid sequence in combination. FIG. 3 depicts a more detailed depiction of the lower panels of FIG. 2. In FIG. 3, the Cas protein and nucleic acid comprising a DNA-binding domain are shown with granularity. The nucleic acid secondary structure is shown that associates with the Cas protein and the viral target DNA. The lower strand of viral DNA shows a cleavage event that disrupts the packaging and transcription of the viral genome, resulting in less functional virus being released by the infected cell.

Results are compiled and analyzed for statistically significant differences between test groups.

Conclusions: Treatment comprising administering P41 VP2 and P2400 VP2 via cell transfection into porcine macrophages results in efficient transcription of CRISPR System components from each vector. Prophylactic treatment with both P41 VP2 and P2400 VP2 results in a decrease in the percentage of porcine macrophages infected with ASFV following a subsequent exposure to ASFV. Concurrent treatment or treatment after exposure to infectious ASFV results in a decrease in the percentage of porcine macrophages showing active ASFV infection.

### Example 2: Prophylaxis and Therapeutic Benefit of CRISPR-Cas12a Targeting of ASFV

In Vitro Testing: For cell culture, in a 12 well plate, HEK 293T cells were cultured at a seeding concentration of 5×10⁵ live cells per well and grown to between 20-70% confluence for testing.

The following plasmids were used for cell transfection in this study. Plasmids used in this study contained a cas gene under a CMV/chicken-beta-actin promotor and a crRNA(s) under a U6 promotor and are labeled as follows: 1) P18 VP2 (Cas9_ΔNLS_GFP)pCas9 ASFV scrambled target, 3 sgRNA that do not target anything (thereby serving as a negative control); 2) P41 VP2 (Cas12a2_GFP) Cas12A2_DP_RH_TI, Cas12a2 with sgRNAs (duplicate) targeting three ASFV genes: DNA polymerase, RNA helicase, and topoisomerase II; 3) P2400 VP2 (Cas12a2_ΔNLS_GFP) p41_Cas12a2_DeltaNSL, NLS deletion Cas12a2 with sgRNAs (duplicate) targeting three ASFV genes: DNA polymerase, helicase, and Topoisomerase II. Target sites were selected within ASFV topoisomerase, polymerase, and helicase genes adjacent to valid PAM/PFS sequences. Thus, additional description of the plasmids used in this Example is as follows: 1) P18 VP2 serves as a negative control with scrambled Guide RNA sequences directing the targeting of Cas9 without an NLS; P18 VP2 does contain Cas but is not targeted to ASFV DNA or ASFV RNA indicating its utility as a control. 2) P41 VP2 targets DNA encoding ASFV topoisomerase, polymerase, and helicase genes, and ssRNA complementary to one of strands of DNA encoding ASFV topoisomerase, polymerase, and helicase genes with duplicate Guide RNA sequences directing the targeting of Cas12a2 comprising an NLS to those sequences; and 3) P2400 VP2 targets DNA encoding ASFV topoisomerase, polymerase, and helicase genes, and ssRNA complementary to one of strands of DNA encoding ASFV topoisomerase, polymerase, and helicase genes with duplicate Guide RNA sequences directing the targeting of Cas12a2 that does not comprise a linked NLS to those sequences. Five groups of HEK293T cells are tested (Group 1: MOCK; Group 2: untransfected control; Group 3: P18 VP2; Group 4: P41 VP2; and Group 5: P2400 VP2).

Transfection: HEK 293T cells were plated in a 12 well plate for 24 hours at 37° C. before transfection. The CRISPR-Cas plasmids were transfected as follows: Lipofectamine 3000 reagent was diluted in Opti-MEM medium into two tubes. In a separate tube, a DNA master mix was created by mixing plasmid DNA with Opti-MEM medium and P3000 Reagent. The DNA master mix was added to each tube containing lipofectamine 3000/Opti-MEM. After 15 min at room temperature, transfection mixtures were added to cultured HEK 293T cells in a 12 well plate and the cells were incubated for 24 hours at 37° C. 24 hours post transfection with plasmids, cells were infected with ASFV E70MS at MOI 1 without inoculum and incubated for an additional 48 hours at 37° C. After 28 hours the infected cells were collected and were titrated with an antibody pair. The primary antibody was specific to ASFV protein P72, and the secondary antibody was specific to the primary antibody and was conjugated with BV421. The fluorescence of BV421 was analyzed using flow cytometry. Plasmids used: P18 which is a control, does contain Cas but not targeting. P41 VP2 and P2400 VP2 were also used.

Gathering Data: The cells were then collected, fixed, and incubated with anti-P72 unconjugated/anti IgG1-BV421 antibodies in preparation for analysis using flow cytometry.

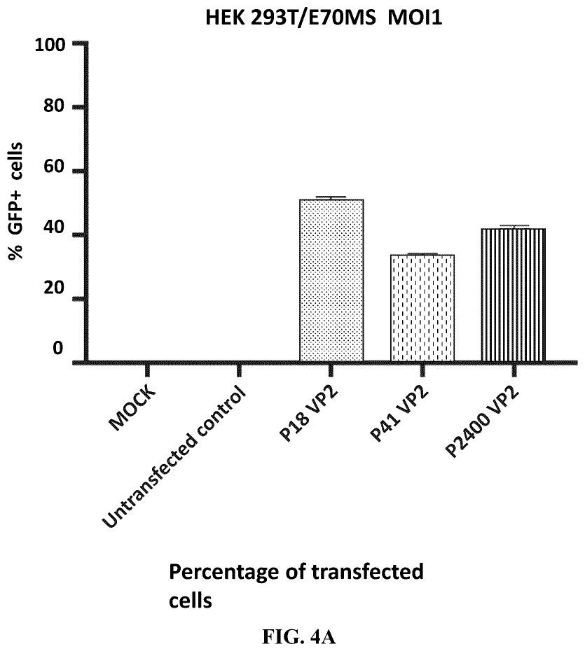

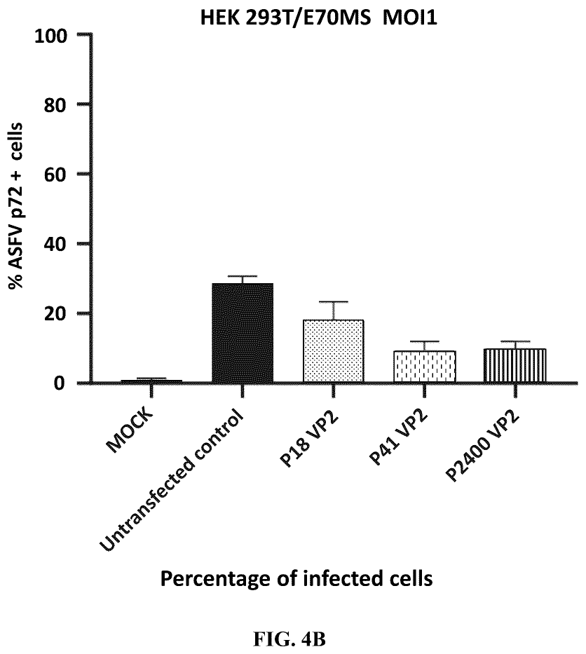

The CRISPR-Cas therapeutic against ASFV was designed to target essential replication genes of ASFV dsDNA genome and RNA transcripts. To evaluate the feasibility of our approach, cell culture experimentation in HEK293T cells was used. The HEK293T cells were seeded in 12 well plates for 24 hours at an initial seeding concentration of 5×10⁵ live cells/well. After 24 hours the plasmids (p18 VP2, P41 VP2, or P2400 Vp2) were delivered by transfection using lipofectamine 3000. 24 hours after the transfection, the cells were infected with ASFV E70MS at an MOI of 1. To determine therapeutic transfection efficiency, transfected plasmids contained the gene for green fluorescent protein (GFP). Successfully transfected cells expressed GFP and were quantified using flow cytometry. Flow Cytometry uses lasers as a light source and creates scattered and fluorescent light that can be measured. The signals are converted into electrical signals that are used to count cell populations, allowing measurement of how many cells contain GFP or another fluorescent marker. Based upon this analysis the therapeutic transfection efficiency was calculated at approximately 55%, 35% and 42% for P18 VP2, P41 VP2, and P2400 VP2 respectively (FIG. 4A). This specifically indicated that Cas protein was being expressed. GFP is linked to the Cas protein, since GFP was being expressed the Cas protein was likewise being expressed.

### Viral Load: Transfected Versus Untransfected

To quantify the viral load, the percentage of cells infected with ASFV was determined by using immunofluorescence detection. Specifically, cells were titrated with two antibodies: an antibody specific to the ASFV protein P72 and an antibody specific to the first antibody that was labeled with a fluorescent molecule called a fluorochrome. By utilizing these antibodies, cells infected with ASFV were effectively labeled with a fluorescent molecule that can be detected using flow cytometry. To rule out the possibility of the antibodies binding to cells not infected with ASFV and giving a false positive result, non-infected cells were titrated with antibodies and obtained flow cytometry data. The results indicate that without ASFV present, only 0.78% of cells are labeled with the antibodies (FIG. 4B, Mock). Next, the viral load of ASFV infected cells was measured. Cells that were not administered a therapeutic were found on average 28.6% of cells were infected with ASFV.

### Viral Load: Cas9 Plasmid

Cells were transfected with a CRISPR-Cas9 prophylactic (P18 VP2) prior to infection with ASFV, the average percentage of cells infected with ASFV decreased to 18% (FIG. 4B, P18 VP2), which indicates a reduction in viral load of 37% (FIG. 4B). It should be noted that this CRISPR-Cas9 therapeutic did not contain a nuclear localization signal (NLS) and therefore may not have been significantly concentrated in the nucleus of treated cells.

### Viral Load: Cas12a Plasmid

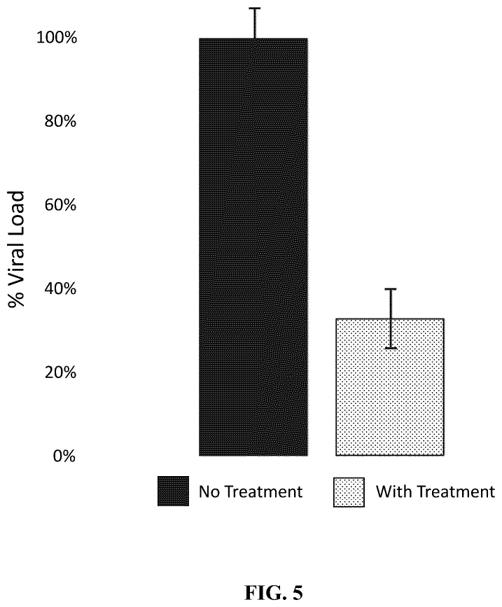

An alternative therapeutic strategy tested was to use the RNA targeting CRISPR-Cas protein Cas12a2 to target the RNA transcripts of ASFV, the targeted DNA polymerase, helicase and Topoisomerase II. When cells were transfected with the CRISPR-Cas12a2 prophylactic prior to infection with ASFV the final viral load was reduced to 9.4% and 10% for Cas12a2 with and without an NLS respectively (FIG. 4B), which indicates a reduction in viral load of approximately 67% and 65% using Cas12a2 with and without an NLS respectively (FIG. 4B and FIG. 5). FIG. 5 depicts results from this in vitro experiment that normalized the number of ASFV-infected cells treated and untreated with Cas protein and nucleic acid sequences comprising viral DNA targeting domains. Viral Load Percentage was calculated for nontreated and treated cells. The results depict approximately a 65% viral load reduction in treated experimental Group 5 of transfected cells treated with a nucleic acid vector of P2400 VP2. The similarity of results obtained using Cas12a2 with and without an NLS further supports a conclusion that concentrating a CRISPR-Cas therapeutic within the nucleus by targeting the CRISPR-Cas therapeutic to the nucleus of the cell is not necessary in the case of ASFV to successfully provide a beneficial prophylactic effect. Therefore, an NLS sequence coupled to a Cas protein is not required for this application of CRISPR to treat ASFV.

Conclusion: The use of a CRISPR-Cas prophylactic against ASFV resulted in a significant decrease in viral load in mammalian cells. By directly targeting the viral genome, the CRISPR-Cas therapeutic mutates essential replication genes to prevent viral replication. While targeting the viral transcripts, the CRISPR-Cas therapeutic prevents viral gene expression. By using a multiplexed design, multiple locations are targeted at once, which reduces the chance of the virus evading the therapeutic agent through mutations. The findings highlight CRISPR-Cas as a viable therapeutic strategy for the prophylaxis and treatment of African Swine Fever.

### Example 3: In Vivo Experiments

### Materials and Methods:

Pigs are to be housed in an isolation unit under BSL3. The pigs are to be fed once per day with a concentrated feed with water ad libitum. According to the European legislation on the protection of animals used for scientific purposes (Directive 2010/63/EU), maintenance and experimental protocols are to be established. Furthermore, an application is forwarded for the request for animal experiments (D. Lgs. 26/2014) to the Italian Ministry of Health.

Test animals: The trial uses 8 to 12-week-old pigs (22-40 kg) with five animals per treatment (Table 4). Smaller pig size reduces the amount of therapeutic required and aids in manageability (size as well as access for easy IV injections) of the animals.

Table 4—Description of the different treatment and control groups

### Experiment 1

| | | Number of | Viral |
|---|---|---|---|
| # | Treatment | Pigs (n) | challenge |
| 1 | Control, positive | 5 | YES |
| 2 | Multiple gene target vector 1 | 5 | YES |
| 3 | Multiple gene target vector 1 | 5 | NO |

### Experiment 2

| | | Number of | Viral |
|---|---|---|---|
| # | Treatment | Pigs (n) | challenge |
| 1 | Control, positive | 5 | YES |
| 2 | Multiple gene target vector 2 | 5 | YES |
| 3 | Multiple gene target vector 2 | 5 | NO |

All animals are to be ear-tagged and divided into treatment and control groups. Each group is housed in separate pens within the same room to reduce external and/or environmental variability. No equipment is shared between groups. All animals are pre-screened free of disease and antibodies against ASF, or other evidence of prior ASFV history. Additionally, all animals are screened free of other viruses virologically (e.g., ASFV, PRRS).

Operational Phases: This is the first phase of testing in vivo. The highest priority is to validate that gene editing vectors can impact the progression of ASF in swine. Listed below are the operational phases.
Phase 1—Acclimate swine to BSL3 pens
Phase 2—Treatment & Control Dosing
Phase 3—Injection challenge with ASFV
Phase 4—Morbidity & Mortality monitoring with associated sample collection
Phase 5—Termination and Necropsy
Phase 6—Sample analysis
Phase 7—Data analysis
Phase 8—Final report

Sampling: Animals are acclimated to their environment for seven days following the European guidelines. Pig temperatures are taken, and clinical scoring performed daily based on the attached scoring/temperature sheets and criteria for routine health changes. At the end of the acclimation period (treatment day 0), all animals are IV-injected with the therapeutic agent with a formulation of pDNA in a lipid nanoparticle (or chitosan carrier). On day 2, post-treatment animals receive a 2^{nd} IV injection of the therapeutic in addition to a low dose of high virulence genotype II ASFV′. Animals are monitored for any health changes daily, and blood samples (into EDTA tubes) are collected on Days 2, 4, 7, and 12 post infection (p.i.) to determine: 1) viral load and 2) expression of therapeutic components by PCR. Two tubes of blood samples are taken for each pig. The first tube is used to determine viral load via PCR. This tube is stored on ice until aliquoted or stored at −80° C. until processed. The second tube is used for RNA preparation. These samples are used for real-time RT-PCR to determine expression of the gene editing components (Cas9 and the sgRNAs). These samples are also used to determine specific mRNAs of the virus. The gene editing components to be measured are Cas9 (via both qPCR and immunoblotting) and the sgRNAs (via qPCR). The ASFV mRNAs are detected by real-time RT-PCR and target the genes being targeted by the vectors being used. Gene targets comprise: DNA polymerase, Topoisomerase II, and RNA helicase, that may or may not be included in the multiplexed vectors as tested. Routine monitoring of health changes occurs daily. Pigs that have survived have blood and serum samples taken and then be euthanized on Day 14 p.i. Serawill is used directly for ELISA test against ASFV. Tissue samples from all experimental groups (especially spleen and lymph nodes) are collected to determine foci of infection and any side effect of the therapeutic treatment. Non-invasive sampling techniques based on the analysis of oral fluid specimens are used for animal disease detection for which effective population level sampling methods may not be available. A rope-in-a-bait based oral fluid sampling technique is used to detect ASFV nucleic acid shedding for experimentally infected domestic pigs or in testing of wild boar populations for transboundary animal disease detection. The method used in the rope-in-a-bait based oral fluid sampling technique is described in (Dietze K et al. Rope-based oral fluid sampling for early detection of classical swine fever in domestic pigs at group level. BMC Vet Res. 2017 Jan. 5; 13(1):50) which is herein incorporated by reference in regard to methods of rope-based oral fluid sampling. Specific detailed postmortem exams and images are conducted by a trained pathologist to search for ASF lesions and other pathological changes after each mortality or euthanasia event. A final necropsy is performed at the end of the study on all remaining animals. Postmortem analysis is used to document ASFV changes and collect tissues (muscles, lung, spleen, kidney, lymph-nodes) for histopathology in addition to any negative indications of the therapy.

Collected tissues will be processed for confirmatory histopathology to validate that the pigs have lesions associated with ASF as well as that the treatment with the Cibus therapeutic did not generate unexpected negative impact on the pigs. Table 5 shows schedule for different sample collection and action.

TABLE 5
In vivo test schedule and action

| Days | 0 | 2 | 4 | 7 | 12 | 14 | Testing |
|---|---|---|---|---|---|---|---|
| Treatment | X | X | | | | | |
| Challenge | | X | | | | | |
| Serum | | | | | | X | ASFV ELISA |
| collection | | | | | | | |
| EDTA | | X | X | X | X | X | ASFV quantitative Real- |
| blood | | | | | | | Time PCR (qRT-PCT) |
| collection | | | | | | | Real Time for Immune |
| | | | | | | | gene |
| | | | | | | | expression |
| Tissue | | | | | | X | Real Time for Immune |
| Collection | | | | | | | gene |
| Necropsy | | | | | | | expression |
| | | | | | | | Histopathology |
| Euthanasia | | | | | | X | |

Viral Load: Viral load is determined using high-quality bead DNA extraction followed by quantitative PCR (qPCR).

Immune gene expression: mRNA content is determined using real-time RT-PCR. Dixon, Islam et al. (2019) outline several potential genes that are up and down regulated by ASFV infection. These include IRF3 & IFN-beta (downregulated), TNF-alpha (downregulated to prevent apoptosis), and NFk-beta (activated by A224L). Zhu, Ramanathan et al. (2019) have a complete transcriptome of ASFV infected macrophages that outline all the up and down regulated genes.

Validation of Cas9 and sgRNA: Cas9 expression and sgRNA production is validated by real-time RT-PCR. This is done with the blood samples as well as the tissues sampled on day 14. Primers that bracket the sgRNA cassette are used for the PCR to examine expression of sgRNAs. Often these are concatemers and, therefore, three amplicons form—reflected in three melting points.

Histopathology: Tissue samples are paraffin embedded and shipped for histopathology analysis.

### Timeline:

| Week | 0-2 | 2-4 | 4-6 | 6-8 | 8-10 | 11-12 |
|---|---|---|---|---|---|---|
| Live swine trial | X | | | | | |
| Sample analysis & | | X | X | X | X | |
| data work-up | | | | | | |
| Final report | | | | | | X |

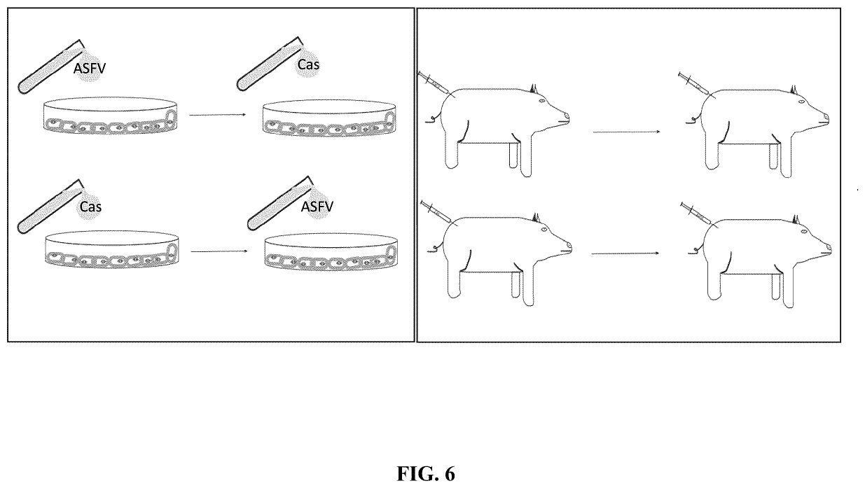

FIG. 6 depicts two panels, a left and right panel showing prophetic experiments to be performed. The left panel depicts two sets of in vitro experiments in which a first set shows steps involved in a method of treating or exposing porcine cells to Cas protein/nucleic acid embodiments after exposure to ASFV. This schematic depicts an experiment to identify dosage for therapeutically effective amounts of treatment. The second set of experiments depicts steps involved in a method of treating or exposing porcine cells to Cas protein/nucleic acid embodiments before exposure to ASFV, which can identify dosage for prophylactically effective amounts of treatment. The right-hand side of experiments shows an identical series of steps except in actual animals (e.g., pigs), rather than porcine cells.

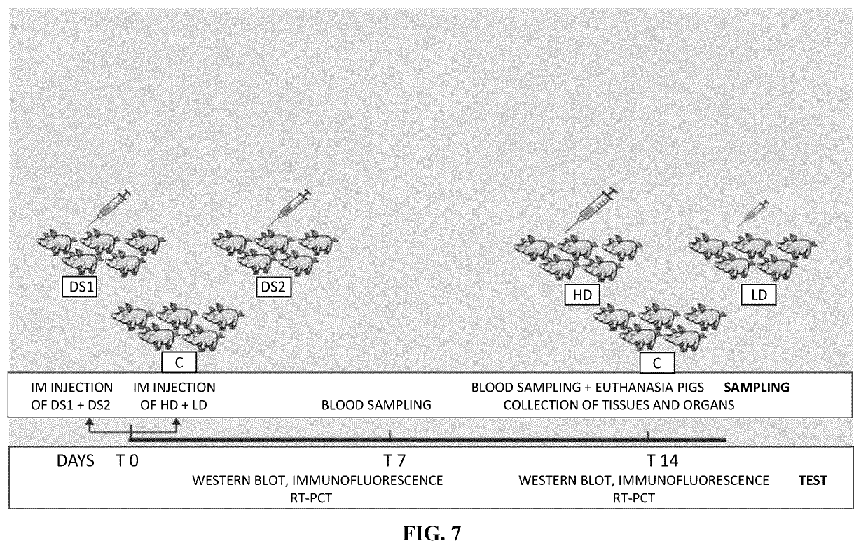

FIG. 7 depicts two sets of experiments used to identify treatment dosage in pigs as well as a timeline for running the experiments. As shown in FIG. 7 on the left, 15 Pigs are injected intramuscularly with Cas protein and nucleic acid embodiments at a dose 1 (DS1) or dose 2 (DS2). As shown in FIG. 7 on the right, 15 Pigs are injected intramuscularly with Cas protein and nucleic acid embodiments at a high dose (HD) or low dose (LD). Blood sampling is performed after therapeutic compositions are administered to the pigs to monitor viral load as compared to control animals (C) which are untreated with therapeutic compositions.

Resources Needed: BSL3 facilities are needed for doing whole swine study (15 pigs for one experiment). BSL3 facilities for processing samples for analytical studies (necropsy+virology+serology+histopathology) are needed. Real-time RT-PCR reagents and instrument—to look for expression of editing machinery as well as representative viral genes and immune genes. Real-time PCR reagents and instruments—to look for presence of ASFV and for viral load analysis are needed. A necropsy suit is needed.

### Aspects:

Some embodiments relate to any of the following aspects:

Aspect 1: A method of treating African Swine Fever in a subject in need thereof, the method comprising administering to the subject a therapeutically effective amount of a first nucleic acid sequence, or a pharmaceutically acceptable salt thereof; wherein the first nucleic acid sequence comprises a nucleic acid sequence that comprises at least about 70% complementary to one or more endogenous African Swine Fever Virus (ASFV) nucleic acid sequences.

Aspect 2: The method of aspect 1 wherein the one or more endogenous ASFV nucleic acid sequences encodes an ASFV helicase, an ASFV DNA polymerase, or an ASFV topoisomerase, or any combination thereof

Aspect 3: The method of aspect 1 or 2, wherein the first nucleic acid sequence comprises at least about 70% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45.

Aspect 4: The method of any one of aspects 1-3, wherein the subject is a pig or minipig.

Aspect 5: The method of any one of aspects 1-4, wherein the nucleic acid sequence comprises at least about 80% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14, SEQ ID NO:15, or a functional fragment thereof.

Aspect 6: The method of any of aspects 1-5, wherein the nucleic acid sequence is a functional fragment comprising at least about 75% sequence identity to SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14, or SEQ ID NO:15.

Aspect 7: The method of any one of aspects 1-6, wherein the subject is a mammal.

Aspect 8: The method of any one of aspects 1-7, wherein the subject has been diagnosed with a need for treatment of the ASFV prior to the administering step.

Aspect 9: The method of claim 1, wherein the effective amount is a prophylactically effective amount.

Aspect 10: A method of inhibiting ASFV replication or assembly in a cell comprising administering to the subject a therapeutically effective amount of a nucleic acid sequence comprising at least about 80% sequence identity to one or a combination of the nucleic acid sequences of Table 2.

Aspect 11: A pharmaceutical composition comprising a therapeutically effective amount of a nucleic acid sequence comprising at least one or a combination of domains from a 5′ to 3′ orientation: a DNA-binding domain, a Cas protein-binding domain, and a transcription terminator domain, wherein the DNA-binding domain comprises from about 1 to about 50 ribonucleotides or deoxyribonucleotides complementary to an ASFV DNA target sequence; and a pharmaceutically acceptable carrier.

Aspect 12: The pharmaceutical composition of aspect 11, wherein ASFV DNA target sequence is about 70% sequence identity to a nucleic acid sequence complementary to ASFV helicase 1, helicase 2, helicase 3, topoisomerase 1, topoisomerase 2, topoisomerase 3, or polymerase.

Aspect 13: The pharmaceutical composition of aspect 11 or 12, further comprising a Cas-binding protein comprising at least about 70% sequence identity to Cas9, Cas12(a), Cas12a2, Cas13(d), or a functional fragment thereof.

Aspect 14: The pharmaceutical composition of any one of aspects 11-13, further comprising a nanoparticle, wherein the nanoparticle comprises an interior volume defined by a lipid layer comprising an interior and an exterior surface, wherein the interior volume comprises: (i) a therapeutically effective amount of a nucleic acid sequence comprising at least one or a combination of domains from a 5′ to 3′ orientation: a DNA-binding domain, a Cas protein-binding domain, and a transcription terminator domain, or a pharmaceutically acceptable salt thereof; or (ii) a therapeutically effective amount of a nucleic acid sequence comprising at least one or a combination of domains from a 5′ to 3′ orientation: a DNA-binding domain, a Cas protein-binding domain, and a transcription terminator domain, or a pharmaceutically acceptable salt thereof; and a Cas protein or a nucleic acid sequence encoding a Cas protein, or a pharmaceutically acceptable salt thereof.

Aspect 15: The pharmaceutical composition any one of aspects 11-14, wherein the pharmaceutical composition is in a liquid dosage form.

Aspect 16: The pharmaceutical composition of aspect 15, in a dosage from about 1 to about 1000 micrograms.

Aspect 17: The pharmaceutical composition of any one of aspects 11-16, wherein the nucleic acid comprises at least about 80% sequence identity SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14, SEQ ID NO: 15 or a pharmaceutically acceptable salt thereof.

Aspect 18: A kit comprising a nucleic acid disclosed herein, or a pharmaceutically acceptable salt thereof, and one or more selected from: an agent known for treating inflammation; instructions for treating an ASFV infection; and instructions for administering the saxiphilin in connection with treating an ASFV infection.

1-79. (canceled)

80. A method for treating African Swine Fever in a subject in need thereof, the method comprising administering to the subject a therapeutically effective amount of a composition comprising i) a first nucleic acid sequence that comprises at least about 70% complementary to an endogenous African Swine Fever Virus (ASFV) nucleic acid sequence encoding an ASFV helicase, an ASFV DNA polymerase, or an ASFV topoisomerase; and ii) a second nucleic acid sequence encoding a Cas endonuclease protein or functional fragment thereof comprising Cas12a2.

81. The method of claim 80, wherein the second nucleic acid sequence comprises a nuclear localization signal (NLS).

82. The method of claim 80, wherein the first nucleic acid sequence encodes a sequence having at least 80% sequence identity to any one of SEQ ID NOs: 1, 5-13, or 35-43.

83. The method of claim 82, wherein the first nucleic acid sequence encodes the sequence of any one of SEQ ID NOs: 1, 5-13, or 35-43.

84. The method of claim 80, wherein the method results in a decrease in ASFV viral load in the subject of at least about 30% within less than one week after the administering to the subject the therapeutically effective amount of the first nucleic acid.

85. The method of claim 80, wherein the ASFV virus being treated in the subject belongs to the family Asfarviridae.

86. The method of claim 80, wherein the first nucleic acid sequence encodes for a first guide RNA (gRNA).

87. The method of claim 80, wherein the first nucleic acid is transcribed into the first gRNA, and wherein the first gRNA activates the Cas endonuclease protein or functional fragment thereof.

88. The method of claim 87, wherein the first gRNA is co-expressed with the Cas endonuclease protein or functional fragment thereof.

89. The method of claim 80, wherein the composition comprises a third nucleic acid sequence encoding for a second gRNA.

90. The method of claim 89, wherein the composition comprises a fourth nucleic acid sequence encoding for a third gRNA.

91. The method of claim 90, wherein subsequent to the administering to the subject the composition, the first nucleic acid sequence, the third nucleic acid sequence, or the fourth nucleic acid sequence is transcribed into the first gRNA, the second gRNA, or the third gRNA, respectively.

92. The method of claim 90, wherein the first gRNA, the second gRNA or the third gRNA activate the Cas endonuclease protein or functional fragment thereof.

93. The method of claim 92, wherein the Cas endonuclease protein or functional fragment thereof is co-expressed with the first gRNA, the second gRNA, and the third gRNA.

94. The method of claim 80, wherein the Cas endonuclease protein or functional fragment thereof functions as an endonuclease to cleave or digest the endogenous ASFV nucleic acid sequence or a portion thereof.

95. The method of claim 80, wherein the ASFV topoisomerase comprises an ASFV Topoisomerase II, the ASFV helicase comprises an ASFV RNA helicase, the ASFV DNA polymerase comprises a G1211R ASFV DNA polymerase or functional fragment thereof.

96. The method of claim 95, wherein the Topoisomerase II is P1192R or a fragment thereof.

97. The method of claim 95, wherein the RNA helicase is QP509L, A859L, F105L, B92L, D1133LK, or Q706L.

98. The method of claim 80, wherein the subject is a pig, a swine, a minipig, or a wild boar.

99. The method of claim 80, wherein the first nucleic acid sequence and the second nucleic acid sequence are transcribed from a single vector.
